SEQUENCE LISTING (23 sequences)

SEQ ID NO: 1 - protein, 4 aa, synthetic construct
GGGG

SEQ ID NO: 2 - protein, 5 aa, synthetic construct
GGGGG

SEQ ID NO: 3 - protein, 6 aa, synthetic construct
GGGGGG

SEQ ID NO: 4 - protein, 7 aa, synthetic construct
GGGGGGG

SEQ ID NO: 5 - protein, 8 aa, synthetic construct
GGGGGGGG

SEQ ID NO: 6 - protein, 5 aa, synthetic construct
  VARIANT 1..5: note = can be present in repeats of 1-5
GGGGS

SEQ ID NO: 7 - protein, 10 aa, synthetic construct
GGGGSGGGGS

SEQ ID NO: 8 - protein, 15 aa, synthetic construct
GGGGSGGGGSGGGGS

SEQ ID NO: 9 - protein, 10 aa, synthetic construct
GGGGSGGGGS

SEQ ID NO: 10 - protein, 15 aa, synthetic construct
GGGGSGGGGSGGGGS

SEQ ID NO: 11 - protein, 5 aa, synthetic construct
TKGPS

SEQ ID NO: 12 - protein, 7 aa, synthetic construct
GQPKAAP

SEQ ID NO: 13 - protein, 10 aa, synthetic construct
GGSGSSGSGG

SEQ ID NO: 14 - protein, 5 aa, synthetic construct
TVAAP

SEQ ID NO: 15 - protein, 5 aa, synthetic construct
QPKAA

SEQ ID NO: 16 - protein, 5 aa, synthetic construct
QRIEG

SEQ ID NO: 17 - protein, 7 aa, synthetic construct
ASTKGPS

SEQ ID NO: 18 - protein, 7 aa, synthetic construct
RTVAAPS

SEQ ID NO: 19 - protein, 7 aa, synthetic construct
GQPKAAP

SEQ ID NO: 20 - protein, 5 aa, synthetic construct
DKTHT

SEQ ID NO: 21 - protein, 7 aa, synthetic construct
GQPKAAP

SEQ ID NO: 22 - protein, 6 aa, synthetic construct
TKGPSR

SEQ ID NO: 23 - protein, 7 aa, synthetic construct
HIDSPNK

(19) United States
(12) Patent Application Publication
(10) Pub. No.: US 2024/0018201 A1
(43) Pub. Date: Jan. 18, 2024

(54) METHODS FOR PURIFICATION OF MULTI-SPECIFIC ANTIBODIES

(71) Applicant: Sanofi, Paris (FR)

(72) Inventors: Ercole RAO, Morfelden-Walldorf (DE); David J. Reczek, Sudbury, MA (US); Tingwan Sun, Belmont, MA (US); Lena Wahl, Idstein (DE)

(21) Appl. No.: 18/331,694

(22) Filed: Jun. 8, 2023

Related U.S. Application Data
(60) Provisional application No. 63/350,255, filed on Jun. 8, 2022.

(30) Foreign Application Priority Data
Sep. 8, 2022 (EP) 22315206.7

(51) Int. Cl. C07K 14/47; C07K 1/20

(52) U.S. Cl. CPC C07K 14/4702; C07K 1/20; C07K 2317/31; A61K 38/00

(57) ABSTRACT

Provided are multivalent binding proteins comprising four polypeptide chains, wherein a first heavy chain polypeptide and a first light chain polypeptide associate to form one or more antigen binding domains and a second heavy chain polypeptide and a second light chain polypeptide associate to bind one or more antigen binding domains. Also provided are methods of purifying such multivalent binding proteins.

### CROSS REFERENCE TO RELATED APPLICATIONS

This application claims the priority benefit of U.S. Provisional Application No. 63/350,255, filed Jun. 8, 2022; and European Patent Application No. 22315206.7, filed Sep. 8, 2022, the contents of each of which are incorporated herein by reference in their entirety.

### REFERENCE TO AN ELECTRONIC SEQUENCE LISTING

The contents of the electronic sequence listing (183952034100SEQLIST.xml; Size: 21,251 bytes; and Date of Creation: May 30, 2023) is herein incorporated by references in its entirety.

### FIELD OF THE INVENTION

This disclosure relates to multivalent binding proteins with reduced binding to a Protein L and/or KappaSelect chromatography material, methods of producing such multivalent binding proteins, and methods of purifying such multivalent binding proteins from a composition comprising a multivalent binding protein and an impurity (e.g., a mispaired polypeptide).

### BACKGROUND

The development of multivalent binding proteins (e.g., multivalent and/or multispecific antibodies and antibody constructs) as therapeutic agents for human diseases has great clinical potential. However, production of multivalent binding proteins in IgG format has been challenging, as antibody heavy chains have evolved to bind antibody light chains in a relatively promiscuous manner. As a result of such promiscuous pairing, production of multivalent binding proteins comprising, e.g., two or more antibody heavy chains (or heavy chain constructs) and/or two or more antibody light chains (or light chain constructs) can lead to the formation of undesirable species comprising heavy chain homodimers and/or scrambled of heavy chain/light chain pairs. Chromatographic separation of a correctly assembled multivalent binding protein from such undesirable mispaired species can be difficult, due to the similarities in their structures and molecular masses. Further, complex in vitro assembly reactions and/or purification methods limit the applicability of many multivalent binding protein platforms, especially their use in high-throughput screens necessary for many therapeutic drug pipelines.

There is a need in the art for improved protein purification processes that remove mispaired heavy chain/light chain by-products and increase multivalent binding protein yield.

### BRIEF SUMMARY

In some embodiments, provided is a multivalent binding protein comprising four polypeptide chains that form two antigen binding domains; wherein the four polypeptide chains comprise:
a first heavy chain polypeptide comprising a structure represented by the formula:

VH1-CH1 [I],
a first light chain polypeptide chain comprising a structure represented by the formula:

VL₁-CL₁ [II],
a second heavy chain polypeptide comprising a structure represented by the formula:

VH₂-CH₁ [III],
and a second light chain polypeptide chain comprising a structure represented by the formula:

VL₂-CL₂ [IV];
wherein: VL₁ is a first immunoglobulin light chain variable domain; VL₂ is a second immunoglobulin light chain variable domain, wherein VL₂ is a κ1, κ3, or κ4 subtype light chain variable domain, CL₁ is a first immunoglobulin light chain constant domain, wherein CL₁ is a Cκ subtype light chain constant domain; CL₂ is a second immunoglobulin light chain constant domain; VH₁ is a first immunoglobulin heavy chain variable domain; VH₂ is a second immunoglobulin heavy chain variable domain; CH1 is an immunoglobulin heavy chain constant domain; wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and wherein VH₁ and VL₁ associate to form a first antigen binding domain and VH₂ and VL₂ associate to form a second antigen binding domain.

In some embodiments, provided is a multivalent binding protein comprising four polypeptide chains that form two antigen binding domains; wherein the four polypeptide chains comprise:
a first heavy chain polypeptide comprising a structure represented by the formula:

VH₁-CH1-CH2-CH3 [I],
a first light chain polypeptide comprising a structure represented by the formula:

VL₁-CL₁ [II],
a second heavy chain polypeptide comprising a structure represented by the formula:

VH₂-CH1-CH2-CH3 [III],
and a second light chain polypeptide comprising a structure represented by the formula:

VL₂-CL₂ [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH1, CH2 and CH3 are immunoglobulin heavy chain constant domains; wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and wherein VH₁ and VL₁ associate to form a first antigen binding domain and VH₂ and VL₂ associate to form a second antigen binding domain.

In some embodiments according to (or as applied to) any embodiment herein, the binding of the CL₁ that comprises the one or more substitutions to KappaSelect is reduced by about 90% compared to a CL₁ without the one or more amino acid substitutions. In some embodiments according to (or as applied to) any embodiment herein, binding of the VL₂ that comprises the one or more substitutions to the protein L chromatography material is reduced by about 90% compared to a VL₂ without the one or more amino acid substitutions. In some embodiments according to (or as applied to) any embodiment herein, the one or more amino acid substitutions in the CL₁ that comprises the one or more substitutions is at a position corresponding to 109, 110 or 199, wherein numbering is according to the EU index. In some embodiments according to (or as applied to) any embodiment herein, the one or more amino acid substitutions in the CL₁ that comprises the one or more substitutions is a T109A substitution, a V110D substitution, a Q199K substitution, T109A-V110D substitutions, or T109A-V110D-Q199K substitutions, wherein amino acid numbering is according to the EU index. In some embodiments according to (or as applied to) any embodiment herein, the one or more amino acid substitutions in the CL₁ that comprises the one or more substitutions is at a position corresponding to 109, 198, 199, or 202, wherein numbering is according to the EU index. In some embodiments according to (or as applied to) any embodiment herein, the one or more amino acid substitutions in the CL₁ that comprises the one or more substitutions is a H198R substitution, a Q199W substitution, or T109A-S202R substitutions, wherein amino acid numbering is according to the EU index. In some embodiments according to (or as applied to) any embodiment herein, the one or more amino acid substitutions in the VL₂ that comprises the one or more substitutions is a substitution of a framework amino acid. In some embodiments according to (or as applied to) any embodiment herein, the one or more amino acid substitutions in the VL₂ that comprises the one or more substitutions is at a position corresponding to 12 or 18, wherein numbering is according to Kabat. In some embodiments according to (or as applied to) any embodiment herein, the one or more amino acid substitutions in the VL₂ that comprises the one or more substitutions is a S12P substitution, a R18P substitution, a R18Q substitution, S12P-R18P substitutions, or S12P-R18Q substitutions, wherein numbering is according to Kabat. In some embodiments according to (or as applied to) any embodiment herein, the CH3 domain of the first heavy chain polypeptide and/or the CH3 domain of the second heavy chain polypeptide is a human IgG1 or IgG4 CH3 domain. In some embodiments according to (or as applied to) any embodiment herein, the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V. In some embodiments according to (or as applied to) any embodiment herein, the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W. In some embodiments according to (or as applied to) any embodiment herein, the CH3 of the second heavy chain polypeptide comprises one or more amino acid substitutions that reduce binding to protein A. In some embodiments according to (or as applied to) any embodiment herein, the CH3 of the first heavy chain polypeptide comprises one or more amino acid substitutions that reduce binding to protein A. In some embodiments according to (or as applied to) any embodiment herein, the one or more amino acid substitutions that reduce binding to Protein A are amino acid substitutions at positions corresponding to positions 435 and 436 of human IgG1, wherein numbering is according to the EU Index. In some embodiments according to (or as applied to) any embodiment herein, the amino acid substitutions are H435R and Y436F, wherein amino acid numbering is according to the EU index. In some embodiments according to (or as applied to) any embodiment herein, the CH1, CH2 and CH3 domains of the first heavy chain polypeptide are different from the CH1, CH2 and CH3 domains of the second heavy chain polypeptide. In some embodiments according to (or as applied to) any embodiment herein, the first heavy chain polypeptide is derived from a different species than the second heavy chain polypeptide. In some embodiments according to (or as applied to) any embodiment herein, the first heavy chain polypeptide and the first light chain polypeptide are derived from a mouse heavy chain immunoglobulin and a mouse light chain immunoglobulin, and the second heavy chain polypeptide and the second light chain polypeptides are derived from a rat heavy chain immunoglobulin and a rat light chain immunoglobulin. In some embodiments according to (or as applied to) any embodiment herein, the first heavy chain polypeptide and the second heavy chain polypeptide each comprise an IgG4 CH3 domain. In some embodiments according to (or as applied to) any embodiment herein, the first heavy chain polypeptide comprises a K409R amino acid substitution and the second heavy chain polypeptide comprises a F405L amino acid substitution, wherein numbering is according to the EU index. In some embodiments according to (or as applied to) any embodiment herein, the multivalent binding protein is a bispecific antigen binding protein. In some embodiments according to (or as applied to) any embodiment herein, the first antigen binding domain and the second antigen binding domain bind different antigens.

In some embodiments according to (or as applied to) any embodiment herein, the first heavy chain polypeptide chain of the multivalent binding protein comprises a structure represented by the formula:

VH₁-CH1-CH2-CH3-VH3-L-VL₃ [Ia], and
the second heavy chain polypeptide of the multivalent binding protein comprises a structure represented by the formula:

VH₂-CH1-CH2-CH3 [IIIa],
wherein: VL₃ is a third immunoglobulin light chain variable domain; VH₃ is a third immunoglobulin heavy chain variable domain; L is an amino acid linker; wherein VH₃ and VL₃ associate to form a third antigen binding domain.

In some embodiments according to (or as applied to) any embodiment herein, the first heavy chain polypeptide of the multivalent binding protein comprises a structure represented by the formula:

VH₁-CH1-CH2-CH3-VH₃ [Ib], and
the second heavy chain polypeptide of the multivalent binding protein comprises a structure represented by the formula:

VH₂-CH1-CH2-CH3 [IIIb],
wherein: VH₃ is a third immunoglobulin heavy chain variable domain.

In some embodiments according to (or as applied to) any embodiment herein, the VL₃ comprises one or more amino acid substitutions that reduce binding to the protein L chromatography material compared to a VL₃ without the one or more amino acid substitutions. In some embodiments according to (or as applied to) any embodiment herein, VL₃ is a λ subtype immunoglobulin light chain variable domain or a κ2 immunoglobulin light chain variable domain. In some embodiments according to (or as applied to) any embodiment herein, the multivalent binding protein is bispecific or trispecific. In some embodiments according to (or as applied to) any embodiment herein, the first antigen binding domain, the second antigen binding domain bind, and the third antigen binding domains bind two or three different antigens. In some embodiments according to (or as applied to) any embodiment herein, the first antigen binding domain binds a first antigen, the second antigen binding domain binds a second antigen, and the third antigen binding domain binds a third antigen. In some embodiments according to (or as applied to) any embodiment herein, the first antigen binding domain and the second antigen binding domain bind a first antigen and the third antigen binding domain binds a second antigen.

In some embodiments, provided is a multivalent binding protein comprising four polypeptide chains that form three antigen binding domains; wherein the four polypeptide chains comprise:
a first heavy chain polypeptide comprising a structure represented by the formula:

VH₁-L₃-VH₂-L4-CH₁ [I],
a first light chain polypeptide chain comprising a structure represented by the formula:

VL₂-L₁-VL₁-L₂-CL₁ [II],
a second heavy chain polypeptide comprising a structure represented by the formula:

VH₃-CH1 [III],
and a second light chain polypeptide chain comprising a structure represented by the formula:

VL₃-CL₂ [IV]
wherein: VL₁ is a first immunoglobulin light chain variable domain; VL₂ is a second immunoglobulin light chain variable domain; VL₃ is a third immunoglobulin light chain variable domain; VH₁ is a first immunoglobulin heavy chain variable domain; VH₂ is a second immunoglobulin heavy chain variable domain; VH₃ is a third immunoglobulin heavy chain variable domain; CL₁ is a first immunoglobulin light chain constant domain; CL₂ is a second immunoglobulin light chain constant domain; CH₁ is an immunoglobulin CH₁ heavy chain constant domain; and L₁, L₂, L₃ and L₄ are amino acid linkers; wherein the polypeptide of formula I and the polypeptide of formula II form a cross-over light chain-heavy chain pair; wherein VH₁ and VL₁ associate to form a first antigen binding domain, VH₂ and VL₂ associate to form a second antigen binding domain, and VH₃ and VL₃ associate to form a third antigen binding domain; and wherein:
   a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
   b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
   c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₂ without the one or more amino acid substitutions, and wherein VL₂ is a λ subtype immunoglobulin light chain variable domain or a λ2 subtype immunoglobulin light chain variable domain; or
   d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain.

In some embodiments, provided is a multivalent binding protein comprising four polypeptide chains that form three antigen binding domains; wherein the four polypeptide chains comprise:
a first heavy chain polypeptide comprising a structure represented by the formula:

VH₁-L₃-VH₂-L₄-CH1-CH2-CH3 [Ia],
a first light chain polypeptide chain comprising a structure represented by the formula:

VL₂-L₁-VL₁-L₂-CL₁ [II],
a second heavy chain polypeptide comprising a structure represented by the formula:

VH₃-CH1-CH2-CH3 [IIIa],
and a second light chain polypeptide comprising a structure represented by the formula:

VL₃-CL₂ [IV]
wherein: VL₁ is a first immunoglobulin light chain variable domain; VL₂ is a second immunoglobulin light chain variable domain; VL₃ is a third immunoglobulin light chain variable domain; VH₁ is a first immunoglobulin heavy chain variable domain; VH₂ is a second immunoglobulin heavy chain variable domain; VH₃ is a third immunoglobulin heavy chain variable domain; CL₁ is a first immunoglobulin light chain constant domain; CL₂ is a second immunoglobulin light chain constant domain; CH₁ is an immunoglobulin CH₁ heavy chain constant domain; CH₂ is an immunoglobulin CH₂ heavy chain constant domain; CH3 is an immunoglobulin CH3 heavy chain constant domain; and L₁, L₂, L₃ and L₄ are amino acid linkers; wherein the polypeptide of formula I and the polypeptide of formula II form a cross-over light chain-heavy chain pair; wherein VH₁ and VL₁ associate to form a first antigen binding domain, VH₂ and VL₂ associate to form a second antigen binding domain, and VH₃ and VL₃ associate to form a third antigen binding domain; and wherein:
   a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
   b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
   c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a λ2 subtype light chain variable immunoglobulin domain; or
   d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a λ2 subtype light chain variable immunoglobulin domain.

In some embodiments according to (or as applied to) any embodiment herein, binding protein is trispecific and capable of specifically binding three different antigen targets. In some embodiments according to (or as applied to) any embodiment herein, at least one of L₁, L₂, L₃ or L₄ are each independently 0 amino acids in length. In some embodiments according to (or as applied to) any embodiment herein, L₁, L₂, L₃ or L₄ are each independently at least one amino acid in length.

In some embodiments according to (or as applied to) any embodiment herein, the second heavy chain polypeptide of the multivalent binding protein comprises a structure represented by the formula:

VH₃-L₅-VH₄-L₆-CH1 [IIIb],
and the second light chain polypeptide of the multivalent binding protein comprises a structure represented by the formula:

VL₄-L₇-VL₃-L₈-CL₂ [IVa]
wherein: VL₄ is a fourth immunoglobulin light chain variable domain; VH₄ is a fourth immunoglobulin heavy chain variable domain; L₅, L₆, L₇ and L₈ are amino acid linkers; wherein the polypeptide of formula IIIa and the polypeptide of formula IVa form a cross-over light chain-heavy chain pair; wherein VH₄ and VL₄ associate to form a fourth antigen binding domain; and wherein:
   a) CL₁ comprises one or more amino acid substitutions which reduce binding to a KappaSelect chromatography material compared to a CL₁ without the one or more amino acid substitutions and VL₃ and VL₄ each comprise one or more amino acid substitutions which reduce binding to a protein L chromatography material compared to a VL₃ and VL₄ without the one or more amino acid substitutions;
   b) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions, and wherein VL₄ is a λ subtype immunoglobulin light chain variable domain or a λ2 subtype immunoglobulin light chain variable domain;
   c) CL₁ comprises one or more amino acid substitutions which reduce binding to a KappaSelect chromatography material compared to a CL₁ without the one or more amino acid substitutions and VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a λ2 subtype immunoglobulin light chain variable domain;
   d) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
e) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a λ2 subtype immunoglobulin light chain variable domain; or
   f) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a λ2 subtype immunoglobulin light chain variable domain.

In some embodiments according to (or as applied to) any embodiment herein, the second heavy chain polypeptide of the multivalent binding protein comprises a structure represented by the formula:

VH₃-L₅-VH₄-L₆-CH1-CH2-CH3 [IIIc],
wherein: CH₂ is an immunoglobulin CH₂ heavy chain constant domain and CH3 is an immunoglobulin CH3 heavy chain constant domain. In some embodiments according to (or as applied to) any embodiment herein, the multivalent binding protein is tetraspecific and capable of specifically binding four antigen targets (e.g., four different target antigens).

In some embodiments according to (or as applied to) any embodiment herein, at least one of L₁, L₂, L₃, L₄, L₅, L₆, L₇ or L₈ are each independently 0 amino acids in length. In some embodiments according to (or as applied to) any embodiment herein, L₁, L₂, L₃, L₄, L₅, L₆, L₇ or L₈ are each independently at least one amino acid in length. In some embodiments according to (or as applied to) any embodiment herein, the binding of the CL₁ or the CL₂ that comprises the one or more amino acid substitutions to the KappaSelect chromatography material is reduced by about 90% compared to a CL₁ or a CL₂ without the one or more amino acid substitutions. In some embodiments according to (or as applied to) any embodiment herein, binding of the VL₁, the VL₂, the VL₃ and/or the VL₄ that comprises the one or more substitutions to the protein L chromatography material is reduced by about 90% compared to a VL₁, a VL₂, a VL₃ and/or a VL₄ without the one or more amino acid substitutions. In some embodiments according to (or as applied to) any embodiment herein, the one or more amino acid substitutions in the CL₁ or the CL₂ is at a position corresponding to 109, 110 or 199, wherein numbering is according to the EU index. In some embodiments according to (or as applied to) any embodiment herein, the one or more amino acid substitutions in the CL₁ or the CL₂ that comprises the one or more amino acid substitutions is a T109A substitution, a V110D substitution, a Q199K substitution, T109A-V110D substitutions, or T109A-V110D-Q199K substitutions, wherein amino acid numbering is according to the EU index. In some embodiments according to (or as applied to) any embodiment herein, the one or more amino acid substitutions in the CL₁ or the CL₂ that comprises the one or more substitutions is at a position corresponding to 109, 198, 199, or 202, wherein numbering is according to the EU index. In some embodiments according to (or as applied to) any embodiment herein, the one or more amino acid substitutions in the CL₁ or the CL₂ that comprises the one or more substitutions is a H198R substitution, a Q199W substitution, or T109A-S202R substitutions, wherein amino acid numbering is according to the EU index. In some embodiments according to (or as applied to) any embodiment herein, the one or more amino acid substitutions in the VL₁, the VL₂, the VL₃ and/or the VL₄ that comprises the one or more substitutions is a substitution of a framework amino acid. In some embodiments according to (or as applied to) any embodiment herein, the one or more amino acid substitutions in the VL₁, the VL₂, the VL₃ and/or the VL₄ that comprises the one or more substitutions is at a position corresponding to 12 or 18, wherein numbering is according to Kabat. In some embodiments according to (or as applied to) any embodiment herein, the one or more amino acid substitutions in the VL₁, the VL₂, the VL₃ and/or the VL₄ that comprises the one or more substitutions is a S12P substitution, a R18P substitution, a R18Q substitution, S12P-R18P substitutions, or S12P-R18Q substitutions, wherein numbering is according to Kabat. In some embodiments according to (or as applied to) any embodiment herein, the CH3 domain of the first heavy chain polypeptide and/or the CH3 domain of the second heavy chain polypeptide is a human IgG1 or IgG4 CH3 domain. In some embodiments according to (or as applied to) any embodiment herein, the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH₃ domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V. In some embodiments according to (or as applied to) any embodiment herein, the CH₃ domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W. In some embodiments according to (or as applied to) any embodiment herein, the second heavy chain polypeptide comprises one or more amino acid substitutions that reduce binding to protein A. In some embodiments according to (or as applied to) any embodiment herein, the CH₃ of the first heavy chain polypeptide comprises one or more amino acid substitutions that reduce binding to protein A. In some embodiments according to (or as applied to) any embodiment herein, the one or more amino acid substitutions that reduce binding to Protein A are amino acid substitutions at positions corresponding to positions 435 and 436 of human IgG1, wherein numbering is according to the EU Index. In some embodiments according to (or as applied to) any embodiment herein, the amino acid substitutions are H435R and Y436F, wherein amino acid numbering is according to the EU index.

In some embodiments, provided is a multivalent binding protein comprising four polypeptide chains that form two antigen binding domains; wherein the four polypeptide chains comprise:
a first heavy chain polypeptide comprising a structure represented by the formula:

VH₁-CH1 [I],
a first light chain polypeptide chain comprising a structure represented by the formula:

VL₁-CL₁ [II],
a second heavy chain polypeptide comprising a structure represented by the formula:

VH₂-CL₂ [III],
and a second light chain polypeptide chain comprising a structure represented by the formula:

VL₂-CH1 [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH₁ is an immunoglobulin heavy chain constant domain wherein
   a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
   b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments, provided is a multivalent binding protein comprising four polypeptide chains that form two antigen binding domains; wherein the four polypeptide chains comprise:
a first heavy chain polypeptide comprising a structure represented by the formula:

VH₁-CH1-CH2-CH3 [I],
a first light chain polypeptide chain comprising a structure represented by the formula:

VL₁-CL₁ [II],
a second heavy chain polypeptide comprising a structure represented by the formula:

VH₂-CL₂-CH2-CH3 [III],
and a second light chain polypeptide chain comprising a structure represented by the formula:

VL₂-CH₁ [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH₁ is an immunoglobulin heavy chain constant domain; wherein
   a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
   b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments, provided is a multivalent binding protein comprising four polypeptide chains that form two antigen binding domains; wherein the four polypeptide chains comprise:
a first heavy chain polypeptide comprising a structure represented by the formula:

VH₁-CH1 [I],
a first light chain polypeptide chain comprising a structure represented by the formula:

VL₁-CL₁ [II],
a second heavy chain polypeptide comprising a structure represented by the formula:

VL₂-CH1 [III],
and a second light chain polypeptide chain comprising a structure represented by the formula:

VH₂-CL₂ [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH1, CH2 and CH3 are immunoglobulin heavy chain constant domains; wherein
   a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
   b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments, provided is a multivalent binding protein comprising four polypeptide chains that form two antigen binding domains; wherein the four polypeptide chains comprise:
a first heavy chain polypeptide comprising a structure represented by the formula:

VH₁-CH1-CH2-CH3 [I],
a first light chain polypeptide chain comprising a structure represented by the formula:

VL₁-CL₁ [II],
a second heavy chain polypeptide comprising a structure represented by the formula:

VH₂-CH1-CH2-CH3 [III],
and a second light chain polypeptide chain comprising a structure represented by the formula:

VL₂-CL₂ [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH1, CH2 and CH3 are immunoglobulin heavy chain constant domains; wherein
   a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
   b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments according to (or as applied to) any embodiment herein, the binding of the CL₁ or the CL₂ that comprises the one or more amino acid substitutions to the KappaSelect chromatography material is reduced by about 90% compared to a CL₁ or a CL₂ without the one or more amino acid substitutions. In some embodiments according to (or as applied to) any embodiment herein, the binding of the VL₁ or the VL₂ that comprises the one or more amino acid substitutions to the protein L chromatography material is reduced by about 90% compared to a VL₁ or a VL₂ without the one or more amino acid substitutions. In some embodiments according to (or as applied to) any embodiment herein, the one or more amino acid substitutions in the CL₁ or the CL₂ that comprises the one or more amino acid substitutions is at a position corresponding to 109, 110 or 199, wherein numbering is according to the EU index. In some embodiments according to (or as applied to) any embodiment herein, the one or more amino acid substitutions in the CL₁ or the CL₂ that comprises the one or more amino acid substitutions is a T109A substitution, a V110D substitution, a Q199K substitution, T109A-V110D substitutions, or T109A-V110D-Q199K substitutions. In some embodiments according to (or as applied to) any embodiment herein, the one or more amino acid substitutions in the CL₁ or the CL₂ that comprises the one or more substitutions is at a position corresponding to 109, 198, 199, or 202, wherein numbering is according to the EU index. In some embodiments according to (or as applied to) any embodiment herein, the one or more amino acid substitutions in the CL₁ or the CL₂ that comprises the one or more substitutions is a H198R substitution, a Q199W substitution, or T109A-S202R substitutions, wherein amino acid numbering is according to the EU index. In some embodiments according to (or as applied to) any embodiment herein, the one or more amino acid substitutions in the VL₁ or the VL₂ that comprises the one or more amino acid substitutions is a substitution of a framework amino acid. In some embodiments according to (or as applied to) any embodiment herein, the one or more amino acid substitutions in the VL₁ or the VL₂ that comprises the one or more amino acid substitutions is at a position corresponding to 12 or 18, wherein numbering is according to Kabat. In some embodiments according to (or as applied to) any embodiment herein, the one or more amino acid substitutions in VL₁ or VL₂ that comprises the one or more amino acid substitutions is a S12P substitution, a R18P substitution, a R18Q substitution, S12P-R18P substitutions, or S12P-R18Q substitutions, wherein numbering is according Kabat. In some embodiments according to (or as applied to) any embodiment herein, the CH3 domain of the first heavy chain polypeptide and/or the CH3 domain of the second heavy chain polypeptide is a human IgG1 or IgG4 CH₃ domain. In some embodiments according to (or as applied to) any embodiment herein, the CH₃ domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V. In some embodiments according to (or as applied to) any embodiment herein, the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W. In some embodiments according to (or as applied to) any embodiment herein, the CH3 of the second heavy chain polypeptide comprises one or more amino acid substitutions that reduce binding to a Protein A chromatography material. In some embodiments according to (or as applied to) any embodiment herein, the CH3 of the first heavy chain polypeptide comprises one or more amino acid substitutions that reduce binding to a Protein A chromatography material. In some embodiments according to (or as applied to) any embodiment herein, the one or more amino acid substitutions which reduces binding to the Protein A chromatography material are amino acid substitutions at positions corresponding to positions 435 and 436 of human IgG1, wherein numbering is according to the EU Index. In some embodiments according to (or as applied to) any embodiment herein, the amino acid substitutions are H435R and Y436F, wherein amino acid numbering is according to the EU index. In some embodiments according to (or as applied to) any embodiment herein, the multivalent binding protein is a bispecific antigen binding protein. In some embodiments according to (or as applied to) any embodiment herein, the first antigen binding domain and the second antigen binding domain bind different antigens.

In some embodiments, provided is a multivalent binding protein comprising four polypeptide chains that form four antigen binding domains; wherein the four polypeptide chains comprise:
a first heavy chain polypeptide comprising a structure represented by the formula:

VH₁-CH₁-L₁-VH₂-CH1₂ [I],
a first light chain polypeptide comprising a structure represented by the formula:

VL₁-CL₁-L₂-VL₂-CL₂ [II],
a second heavy chain polypeptide comprising a structure represented by the formula:

VH₃-CH1₃-L₃-VH₄-CH1₄ [III],
a second light chain polypeptide comprising a structure represented by the formula:

VL₃-CL₃-L₄-VL₄-CL₄ [IV]
wherein: VL₁ is a first immunoglobulin light chain variable domain; VL₂ is a second immunoglobulin light chain variable domain; VL₃ is a third immunoglobulin light chain variable domain; VL₄ is a fourth immunoglobulin light chain variable domain; CL₁ is a first immunoglobulin light chain constant domain; CL₂ is a second immunoglobulin light chain constant domain; CL₃ is a third immunoglobulin light chain constant domain; CL₄ is a fourth immunoglobulin light chain constant domain; VH₁ is a first immunoglobulin heavy chain variable domain; VH₂ is a second immunoglobulin heavy chain variable domain; VH₃ is a third immunoglobulin heavy chain variable domain; VH₄ is a fourth immunoglobulin heavy chain variable domain; CH1₁ is a first immunoglobulin heavy chain constant domain; CH1₂ is a second immunoglobulin heavy chain constant domain; CH1₃ is a third immunoglobulin heavy chain constant domain; CH₁₄ is a fourth immunoglobulin heavy chain constant domain; and L₁, L₂, L₃ and L₄ are amino acid linkers; wherein:
   a) CL₁ and CL₂ each comprise one or more amino acid substitutions which reduce binding to a KappaSelect chromatography material compared to a CL₁ and CL₂ without the one or more amino acid substitutions and VL₃ and VL₄ each comprise one or more amino acid substitutions which reduce binding to a protein L chromatography material compared to a VL₃ and VL₄ without the one or more amino acid substitutions;
   b) CL₁ and CL₂ each comprise one or more amino acid substitutions which reduce binding to a KappaSelect chromatography material compared to a CL₁ and CL₂ without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a λ2 subtype immunoglobulin light chain variable domain;
   c) CL₁ and CL₂ each comprises one or more amino acid substitutions which reduce binding to a KappaSelect chromatography material compared to a CL₁ and CL₂ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
   d) CL₁ comprises one or more amino acid substitutions which reduce binding to a KappaSelect chromatography material compared to a CL₁ without the one or more amino acid substitutions, CL₂ is a λ subtype immunoglobulin light chain constant domain, and VL₃ and VL₄ each comprise one or more amino acid substitutions which reduce binding to a protein L chromatography material compared to a VL₁ and VL₂ without the one or more amino acid substitutions;
   e) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
   f) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
   g) CL₂ comprises one or more amino acid substitutions which reduce binding to a KappaSelect chromatography material compared to a CL₂ without the one or more amino acid substitutions, CL₁ is a λ subtype immunoglobulin light chain constant domain, and VL₃ and VL₄ each comprise one or more amino acid substitutions which reduce binding to a protein L chromatography material compared to a VL₃ and VL₄ without the one or more amino acid substitutions;
   h) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
i) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain; and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain.

In some embodiments according to (or as applied to) any embodiment herein, at least one of L₁, L₂, L₃ or L₄ are each independently 0 amino acids in length. In some embodiments according to (or as applied to) any embodiment herein, at least one of L₁, L₂, L₃ or L₄ are each independently at least one amino acid in length.

In some embodiments, provided is a multivalent binding protein comprising four polypeptide chains that form four antigen binding domains; wherein the four polypeptide chains comprise:
a first heavy chain polypeptide comprising a structure represented by the formula:

VH₁-L₁-VH₂-L₂-CH1₁ [I],
a first light chain polypeptide comprising a structure represented by the formula:

VL₁-L₃-VL₂-L₄-CL₁ [II],
a second heavy chain polypeptide comprising a structure represented by the formula:

VH₃-L₅-VH₄-L₆-CH1₂ [III],
a second light chain polypeptide comprising a structure represented by the formula:

VL₃-L₇-VL₄-L₈-CL₂ [IV]
wherein: VL₁ is a first immunoglobulin light chain variable domain; VL₂ is a second immunoglobulin light chain variable domain; VL₃ is a third immunoglobulin light chain variable domain; VL₄ is a fourth immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; VH₃ is a third immunoglobulin heavy chain variable domain; VH₄ is a fourth immunoglobulin heavy chain variable domain; CH1₁i is a first immunoglobulin heavy chain constant domain; CH1₂ is a second immunoglobulin heavy chain constant domain; and L₁, L₂, L₃, L₄ L₅, L₆, L₇ and L₈ are amino acid linkers; wherein:
   a) CL₁ comprises one or more amino acid substitutions which reduce binding to a KappaSelect chromatography material compared to a CL₁ without the one or more amino acid substitutions and VL₃ and VL₄ each comprise one or more amino acid substitutions which reduce binding to a protein L chromatography material compared to a VL₃ and VL₄ without the one or more amino acid substitutions;
   b) CL₁ comprises one or more amino acid substitutions which reduce binding to a KappaSelect chromatography material compared to a CL₁ without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
   c) CL₁ comprises one or more amino acid substitutions which reduce binding to a KappaSelect chromatography material compared to a CL₁ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a λ2 subtype immunoglobulin light chain variable domain;
and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain.

In some embodiments according to (or as applied to) any embodiment herein, at least one of L₁, L₂, L₃, L₄, L₅, L₆, L₇, or L₈ are each independently 0 amino acids in length. In some embodiments according to (or as applied to) any embodiment herein, at least one of L₁, L₂, L₃, L₄, L₅, L₆, L₇, or L₈ are each independently at least one amino acid in length. In some embodiments according to (or as applied to) any embodiment herein, binding of the CL₁ and/or the CL₂—that comprises the one or more amino acid substitutions to the KappaSelect chromatography material is reduced by about 90% compared to a CL₁ and/or a CL₂ without the one or more amino acid substitutions. In some embodiments according to (or as applied to) any embodiment herein, binding of the VL₃ and/or the VL₄ that comprises the one or more amino acid substitutions to the protein L chromatography material is reduced by about 90% compared to a VL₃ and/or a VL₄ without the one or more amino acid substitutions. In some embodiments according to (or as applied to) any embodiment herein, the one or more amino acid substitutions in the CL₁ and/or the CL₂ that comprises the one or more amino acid substitutions is at a position corresponding to 109, 110 or 199, wherein numbering is according to the EU index. In some embodiments according to (or as applied to) any embodiment herein, the one or more amino acid substitutions in the CL₁ and/or the CL₂ that comprises the one or more amino acid substitutions is a T109A substitution, a V110D substitution, a Q199K substitution, T109A-V110D substitutions, or T109A-V110D-Q199K substitutions, wherein amino acid numbering is according to the EU index. In some embodiments according to (or as applied to) any embodiment herein, the one or more amino acid substitutions in the CL₁ and/or the CL₂ that comprises the one or more substitutions is at a position corresponding to 109, 198, 199, or 202, wherein numbering is according to the EU index. In some embodiments according to (or as applied to) any embodiment herein, the one or more amino acid substitutions in the CL₁ and/or the CL₂ that comprises the one or more substitutions is a H198R substitution, a Q199W substitution, or T109A-S202R substitutions, wherein amino acid numbering is according to the EU index. In some embodiments according to (or as applied to) any embodiment herein, the one or more amino acid substitutions in the VL₃ and/or the VL₄ that comprises the one or more amino acid substitutions is a substitution of a framework amino acid. In some embodiments according to (or as applied to) any embodiment herein, the one or more amino acid substitutions in the VL₃ and/or the VL₄ that comprises the one or more amino acid substitutions is at a position corresponding to 12 or 18, wherein numbering is according to Kabat. In some embodiments according to (or as applied to) any embodiment herein, the one or more amino acid substitutions in the VL₃ and/or the VL₄ that comprises the one or more amino acid substitutions is a S12P substitution, a R18P substitution, a R18Q substitution, S12P-R18P substitutions, or S12P-R18Q substitutions, wherein numbering is according to Kabat.

In some embodiments according to (or as applied to) any embodiment herein, the first heavy chain polypeptide of the multivalent binding protein comprises a structure represented by the formula:

VH₁-CH1₁-L₁-VH₂-CH1₂-CH2-CH3 [Ia],
the second heavy chain polypeptide of the multivalent binding protein comprises a structure represented by the formula:

VH₃-CH1₃-L₃-VH₄-CH1₄-CH2-CH3 [IIIa].

In some embodiments according to (or as applied to) any embodiment herein, the first heavy chain polypeptide of the multivalent binding protein comprises a structure represented by the formula:

VH₁-L₁-VH₂-L₂-CH1₁-CH2-CH3 [Ia],
the second heavy chain polypeptide of the multivalent binding protein comprises a structure represented by the formula:

VH₃-L₅-VH₄-L6-CH1₂-CH2-CH3 [IIIa].

In some embodiments according to (or as applied to) any embodiment herein, the CH3 domain of the first heavy chain polypeptide and/or the CH3 domain of the second heavy chain polypeptide is a human IgG1 or IgG4 CH3 domain. In some embodiments according to (or as applied to) any embodiment herein, the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V. In some embodiments according to (or as applied to) any embodiment herein, the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W. In some embodiments according to (or as applied to) any embodiment herein, the CH3 of the second heavy chain polypeptide comprises one or more amino acid substitutions that reduce binding to a Protein A chromatography material. In some embodiments according to (or as applied to) any embodiment herein, the CH3 of the first heavy chain polypeptide comprises one or more amino acid substitutions that reduce binding to a Protein A chromatography material. In some embodiments according to (or as applied to) any embodiment herein, the one or more amino acid substitutions that reduce binding to the Protein A chromatography material are amino acid substitutions at positions corresponding to positions 435 and 436 of human IgG1, wherein numbering is according to the EU Index. In some embodiments according to (or as applied to) any embodiment herein, the amino acid substitutions are H435R and Y436F, wherein amino acid numbering is according to the EU index. In some embodiments according to (or as applied to) any embodiment herein, the binding protein is tetraspecific and capable of specifically binding four different antigen targets.

In some embodiments, provided is a multivalent binding protein comprising four polypeptide chains that form an antigen binding domain; wherein the four polypeptide chains comprise:
a first heavy chain polypeptide that comprises a structure represented by the formula:

VH₁-CH1₁ [I],
a first light chain polypeptide chain that comprises a structure represented by the formula:

VL₁-CL₁ [II],
a second heavy chain polypeptide that comprises a structure represented by the formula:

fusion polypeptide-L₁-CH1₂ [III],
and a second light chain polypeptide chain that comprises a structure represented by the formula:

fusion polypeptide-L₂-CL₂ [IV]
wherein: VL₁ is a first immunoglobulin light chain variable domain; CL₁ is a first immunoglobulin light chain constant domain; CL₂ is a second immunoglobulin light chain constant domain; VH₁ is a first immunoglobulin heavy chain variable domain; CH1₁ is a first immunoglobulin heavy chain constant domain; CH1₂ is a second immunoglobulin heavy chain constant domain; and L₁ and L₂ are amino acid linkers; wherein CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions; and wherein VL₁ and VH₁ form an antigen binding domain.

In some embodiments according to (or as applied to) any embodiment herein, L₁ or L₂ is independently 0 amino acids in length. In some embodiments according to (or as applied to) any embodiment herein, L₁ or L₂ is independently at least one amino acid in length.

In some embodiments, provided is a multivalent binding protein comprising four polypeptide chains that form two antigen binding domains; wherein the four polypeptide chains comprise:
a first heavy chain polypeptide that comprises a structure represented by the formula:

VH₁-CH1₁-L₁-VH₂-CH1₂ [I],
a first light chain polypeptide that chain comprises a structure represented by the formula:

VL₁-CL₁-L₂-VL₂-CL₂ [II],
a second heavy chain polypeptide that comprises a structure represented by the formula:

fusion polypeptide-L₃-CH1₃ [III],
and a second light chain polypeptide that comprises a structure represented by the formula:

fusion polypeptide-L₄-CL₃ [IV]
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; CL₃ is a third immunoglobulin light chain constant domain; VH₁ is a first immunoglobulin heavy chain variable domain; VH₂ is a second immunoglobulin heavy chain variable domain; CH1₁ is a first immunoglobulin heavy chain constant domain; CH1₂ is a second immunoglobulin heavy chain constant domain; CH1₃ is a third immunoglobulin heavy chain constant domain, and L₁, L₂, L₃ and L₄ are amino acid linkers; wherein:
   a) CL₃ comprises one or more amino acid substitutions which reduce binding to a KappaSelect chromatography material compared to a CL₃ without the one or more amino acid substitutions, VL₁ and VL₂ each comprise one or more amino acid substitutions which reduce binding to a protein L chromatography material compared to a VL₁ and VL₂ without the one or more amino acid substitutions,
   b) CL₃ comprises one or more amino acid substitutions which reduce binding to a KappaSelect chromatography material compared to a CL₃ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin domain; or
   c) CL₃ comprises one or more amino acid substitutions which reduce binding to a KappaSelect chromatography material compared to a CL₃ without the one or more amino acid substitutions, VL₂ comprises one or more amino acid substitutions which reduce binding to a protein L chromatography material compared to a VL₂ without the one or more amino acid substitutions, and VL₁ is a λ subtype immunoglobulin light chain variable domain or a κ2 subtype immunoglobulin domain; and
wherein VL₁ and VH₁ form a first antigen binding domain and VL₂ and VH₂ form a second antigen binding domain.

In some embodiments according to (or as applied to) any embodiment herein, at least one of L₁, L₂, L₃, or L₄ are each independently 0 amino acids in length. In some embodiments according to (or as applied to) any embodiment herein, L₁, L₂, L₃ or L₄ are each independently at least one amino acid in length. In some embodiments according to (or as applied to) any embodiment herein, binding of the CL₂ or the CL₃ that comprise the one or more amino acid substitutions to the KappaSelect chromatography material is reduced by about 90% compared to a CL₂ or a CL₃ without the one or more amino acid substitutions. In some embodiments according to (or as applied to) any embodiment herein, binding of the VL₁ and/or the VL₂ that comprise the one or more amino acid substitutions to the protein L chromatography material is reduced by about 90% compared to a VL₁ and/or a VL₂ without the one or more amino acid substitutions. In some embodiments according to (or as applied to) any embodiment herein, the one or more amino acid substitutions in the CL₂ or the CL₃ that comprise the one or more amino acid substitutions is at a position corresponding to 109, 110 or 199, wherein numbering is according to the EU index. In some embodiments according to (or as applied to) any embodiment herein, the one or more amino acid substitutions in the CL₂ or the CL₃ that comprise the one or more amino acid substitutions is a T109A substitution, a V110D substitution, a Q199K substitution, T109A-V110D substitutions, or T109A-V110D-Q199K substitutions. In some embodiments according to (or as applied to) any embodiment herein, the one or more amino acid substitutions in the CL₂ or the CL₃ that comprises the one or more substitutions is at a position corresponding to 109, 198, 199, or 202, wherein numbering is according to the EU index. In some embodiments according to (or as applied to) any embodiment herein, the one or more amino acid substitutions in the CL₂ or the CL₃ that comprises the one or more substitutions is a H198R substitution, a Q199W substitution, or T109A-S202R substitutions, wherein amino acid numbering is according to the EU index. In some embodiments according to (or as applied to) any embodiment herein, the one or more amino acid substitutions in the VL₁ and/or the VL₂ that comprise the one or more amino acid substitutions is a substitution of a framework amino acid. In some embodiments according to (or as applied to) any embodiment herein, the one or more amino acid substitutions in the VL₁ and/or the VL₂ that comprise the one or more amino acid substitutions is at a position corresponding to 12 or 18, wherein numbering is according to Kabat. In some embodiments according to (or as applied to) any embodiment herein, the one or more amino acid substitutions in the VL₁ and/or the VL₂ is a S12P substitution, a R18P substitution, a R18Q substitution, S12P-R18P substitutions, or S12P-R18Q substitutions, wherein numbering is according to Kabat. In some embodiments according to (or as applied to) any embodiment herein, the first heavy chain polypeptide comprises a first CH2 immunoglobulin heavy chain constant domain and a first CH3 immunoglobulin heavy chain constant domain and the second heavy chain polypeptide comprises a second CH2 immunoglobulin heavy chain constant domain and a second CH₃ immunoglobulin heavy chain constant domain. In some embodiments according to (or as applied to) any embodiment herein, the first CH3 domain and/or the CH₃ domain is a human IgG1 or IgG4 CH3 domain. In some embodiments according to (or as applied to) any embodiment herein, the first CH3 domain comprises amino acid substitutions at positions corresponding to positions 354 and 366 of human IgG1, wherein numbering is according to the EU Index, wherein the amino acid substitutions are S354C and T366W; wherein second CH3 domain comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V. In some embodiments according to (or as applied to) any embodiment herein, the CH3 of the second heavy chain polypeptide comprises one or more amino acid substitutions which reduces binding to protein A. In some embodiments according to (or as applied to) any embodiment herein, the CH3 of the first heavy chain polypeptide comprises one or more amino acid substitutions which reduces binding to protein A. In some embodiments according to (or as applied to) any embodiment herein, one or more amino acid substitutions which reduces binding to Protein A are amino acid substitutions at positions corresponding to positions 435 and 436 of human IgG1, wherein numbering is according to the EU Index. In some embodiments according to (or as applied to) any embodiment herein, the amino acid substitutions are H435R and Y436F, wherein amino acid numbering is according to the EU index. In some embodiments according to (or as applied to) any embodiment herein, the multivalent binding protein is a multispecific antibody or antigen binding fragment thereof.

In some embodiments, provided is one or more polynucleotide(s) encoding a multivalent binding protein described herein. In some embodiments, provided is a vector(s) comprising the one or more polynucleotide(s) described herein. In some embodiments, provided is a host cell comprising the one or more polynucleotide(s) or vector(s) described herein. In some embodiments, provided is a method of producing a multivalent binding protein, the method comprising culturing a host cell described herein such that the binding protein is produced (e.g., under conditions where the multivalent binding protein is expressed by the host cell). In some embodiments, the method further comprises recovering the binding protein from the host cell.

In some embodiments, provided is a pharmaceutical composition comprising a multivalent binding protein described herein and a pharmaceutically acceptable carrier.

In some embodiments, provided is a method of purifying a multivalent binding protein provided herein (e.g., separating a multivalent binding protein provided herein from one or more impurities), the method comprising: a) subjecting a composition comprising the multivalent binding protein (e.g., the multivalent binding protein and an impurity, such as mispaired or misassembled polypeptides) to Protein L chromatography in bind and elute mode to generate a protein L eluate, and b) subjecting the protein L eluate to KappaSelect chromatography in bind and elute mode to generate a KappaSelect eluate, wherein the KappaSelect eluate comprises the multivalent binding protein and is essentially free of mispaired (or misassembled) polypeptides. In some embodiments, the multivalent binding protein in the KappaSelect eluate is at least 85% pure, at least 90% pure, or at least 95% pure (e.g., free of mispaired or misassembled polypeptides). In some embodiments, less than 15%, less than 10%, or less than 5% of the polypeptides in the KappaSelect eluate are mispaired (or misassembled) polypeptides.

In some embodiments, provided is a method of purifying the multivalent binding protein described herein (e.g., separating a multivalent binding protein provided herein from one or more impurities, such as mispaired or misassembled polypeptides), the method comprising a) subjecting a composition comprising the multivalent binding protein and mispaired (or misassembled) polypeptides to KappaSelect chromatography in bind and elute chromatography to generate as KappaSelect eluate and b) subjecting the KappaSelect eluate to Protein L chromatography in bind and elute mode to generate a protein L eluate, wherein the protein L eluate comprises the multivalent binding protein and is essentially free of the mispaired (or misassembled) polypeptides. In some embodiments, the multivalent binding protein in the protein L eluate is at least 85% pure, at least 90% pure, or at least 95% pure (e.g., free of mispaired or misassembled polypeptides). In some embodiments, less than 15%, less than 10%, or less than 5% of the polypeptides in the protein L eluate are mispaired or misassembled polypeptides.

In some embodiments, provided is a method of purifying the multivalent binding protein described herein (e.g., separating a multivalent binding protein provided herein from one or more impurities), the method comprising: a) subjecting a composition comprising the multivalent binding protein (e.g., the multivalent antigen binding protein and an impurity, such as a mispaired or misassembled polypeptide) to Protein A chromatography in bind and elute mode to generate a Protein A eluate, b) subjecting the Protein A eluate to Protein L chromatography in bind and elute mode to generate a protein L eluate, and c) subjecting the protein L eluate to KappaSelect chromatography in bind and elute mode to generate a KappaSelect eluate, wherein the KappaSelect eluate comprises the multivalent binding protein and is essentially free of mispaired (or misassembled) polypeptides. In some embodiments, the multivalent binding protein in the KappaSelect eluate is at least 85% pure, at least 90% pure, or at least 95% pure (e.g., free of mispaired or misassembled polypeptides). In some embodiments, less than 15%, less than 10%, or less than 5% of the polypeptides in the KappaSelect eluate are mispaired (or misassembled) polypeptides.

In some embodiments, provided is a method of purifying the multivalent binding protein described herein (e.g., separating a multivalent binding protein provided herein from one or more impurities), the method comprising: a) subjecting a composition comprising the multivalent binding protein (e.g., a composition comprising the multivalent binding protein and mispaired or misassembled polypeptides) to Protein A chromatography in bind and elute mode to generate a Protein A eluate, b) subjecting the Protein A eluate to KappaSelect chromatography in bind and elute mode to generate a KappaSelect eluate, and c) subjecting the protein KappaSelect eluate to Protein L chromatography in bind and elute mode to generate a protein L eluate, wherein the L eluate comprises the multivalent binding protein and is essentially free of mispaired (or misassembled) polypeptides. In some embodiments, the multivalent binding protein in the protein L eluate is at least 85% pure, at least 90% pure, or at least 95% pure (e.g., free of mispaired or misassembled polypeptides). In some embodiments, less than 15%, less than 10%, or less than 5% of the polypeptides in the protein L eluate are mispaired (or misassembled) polypeptides.

In some embodiments according to (or as applied to) any embodiment herein, the composition comprising the multivalent binding protein is derived from a host cell engineered to express the multispecific binding protein. In some embodiments according to (or as applied to) any embodiment herein, the composition comprising the multivalent binding protein is a host cell culture supernatant. In some embodiments according to (or as applied to) any embodiment herein, the composition comprising the multivalent binding protein further comprises mispaired polypeptides. In some embodiments according to (or as applied to) any embodiment herein, the composition comprising the multivalent binding protein is filtered prior to chromatography. In some embodiments according to (or as applied to) any embodiment herein, the method further comprising a polishing step after the KappaSelect or protein L chromatography. In some embodiments according to (or as applied to) any embodiment herein, the polishing step is a size exclusion chromatography. In some embodiments according to (or as applied to) any embodiment herein, the Protein A chromatography is a MabSelect™, MabSelect SuRe™, MabSelect SuRe™ LX, MabSelect PrismA, ProSep®-vA, ProSep® Ultra Plus, Protein A Sepharose® Fast Flow, or Toyopearl® AF-rProtein A chromatography. In some embodiments according to (or as applied to) any embodiment herein, the protein L chromatography is a Pierce™ Protein L chromatography cartridge, a Capto™ L chromatography, HiTrap® Protein L chromatography, a TOYOPEARL® AF-rProtein L-650F chromatography, or a KanCap™ L chromatography. In some embodiments according to (or as applied to) any embodiment herein, the KappaSelect chromatography is a HiTrap™ KappaSelect or a CaptureSelect™ Kappa XL chromatography. In some embodiments according to (or as applied to) any embodiment herein, the composition comprising the multivalent binding protein is combined with a pharmaceutically acceptable carrier.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A is a schematic representation of an exemplary bivalent binding protein.

FIG. 1B is a schematic representation of an exemplary Triomab® bivalent binding protein.

FIG. 1C is a schematic representation of an exemplary Duomab® bivalent binding protein.

FIG. 1D is a schematic representation of an exemplary Ab-Nb trivalent binding protein with a scFv attached to the “knob” heavy chain.

FIG. 1E is a schematic representation of an exemplary Ab-Nb trivalent binding protein with a VHH attached to the “knob” heavy chain.

FIG. 1F is a schematic representation of an exemplary CODV multivalent binding protein comprising a CODV arm on the left and a Fab arm on the right.

FIG. 1G is a schematic representation of an exemplary CODV multivalent binding protein comprising two CODV arms.

FIG. 1H is a schematic representation of an exemplary cross-mab bivalent binding protein where the CL and CH₁ domains of one arm of the binding protein have been swapped.

FIG. 1I is a schematic representation of an exemplary cross-mab bivalent binding protein where the VL and VH domains of one arm of the binding protein have been swapped.

FIG. 1J is a schematic representation of an exemplary tandem Fab multivalent binding protein wherein each variable domain comprises VL, CL, VH and CH1 domains.

FIG. 1K is a schematic representation of an exemplary tandem Fab multivalent binding protein wherein N-terminus variable domains comprise VL and VH domains and the C-terminus variable domains comprise VL, CL, VH and CH1 domains.

FIG. 1L is a schematic representation of an exemplary multivalent binding protein comprising a Fab arm and a fusion protein arm. The Fab arm comprises a single Fab.

FIG. 1M is a schematic representation of an exemplary multivalent binding protein comprising a Fab arm and a fusion protein arm. The Fab arm comprises tandem Fabs.

FIG. 2 is a schematic of a representative three-step process for purifying a CODV multivalent binding protein. The desired product is shown in the dotted boxes. The CODV includes knob and hole substitutions. The knob arm of the CODV includes KappaSelect KO mutations (e.g., mutations that reduce the binding of VL domains to KappaSelect chromatography medium). The hole arm of the CODV includes Protein L KO mutations (e.g., mutations that reduce the binding of CL domains to Protein L) and RF mutations (e.g., H435R and 436F mutations, wherein amino acid numbering is according to the EU Index) of the CH3 domain (see, e.g., Edelman et al., 1969, Proc Natl Acad Sci USA 63: 78-85). Representative mispaired products are shown in the upper left next to the desired product. The three steps in the process include Protein A chromatography (e.g., MabSelectSure®, (MSS)) followed by protein L chromatography followed by KappaSelect (KS) chromatography. Removal of mispaired proteins is shown.

FIG. 3 shows alignment of amino acid sequences of different VL kappa and lambda subtypes. Taken from Graille et al., Structure, Vol. 9, 679-687, August, 2001.

FIG. 4 shows the results of screening potential ProL KO mutations. Top panel shows mutations evaluated. Bottom panel shows results based on protein yield following MabSelect® Sure chromatography.

FIG. 5 shows Biolayer interferometry (BLI) binding assessment of Adalimumab ProL KO variants to ProL ligand.

FIG. 6 shows BLI binding assessment of Adalimumab ProL KO variants to TNFα.

FIG. 7 shows the evaluation of Adalimumab ProL KO variants versus wild-type binding to protein-L Resin.

FIG. 8 shows the results of screening potential KS KO mutations. Top panel shows mutations evaluated. Bottom panel shows results based on protein yield following MabSelect® Sure chromatography

FIG. 9 shows BLI binding assessment of Adalimumab KS KO variants to KS ligand.

FIG. 10 shows BLI binding assessment of Adalimumab KS KO variants to TNFα.

FIG. 11 shows the evaluation of Adalimumab KS KO variants versus wild-type binding to KS Resin.

FIG. 12 shows a schematic of a two-step chromatography of a trispecific CODV containing antibody, harboring a Vk2 Fab arm LC with a proL KO mutations and a Vk1 CODV arm LC capable of pro-L binding. The first step is MSS chromatography. The MSS eluted material containing the triAb of interest as well as 2×FabLC and 2×CODV LC mispaired species is then further purified in a second step over protein-L resin.

FIG. 13 shows Coomassie-stained SDS-PAGE gel of samples from the trispecific CODV 2-step purification.

FIG. 14 shows analytical size exclusion chromatography (aSEC) data of wild type and ProL KO mutant versions of Adalimumab post-MSS purification. Percent of expected main peak is shown.

FIG. 15 shows analysis of samples of WT Adalimumab and the S12P-R18P mutant taken at the end of an accelerated stability test (40° C. for 2 weeks). Top panel shows a Coomassie-stained SDS-PAGE gel of non-reduced and reduced samples. Bottom panels show aSEC data for each respective sample. Percent of expected main peak is shown.

FIG. 16 shows Differential Scanning Fluorimetry (nano-DSF) data and derived Tm for WT Adalimumab and an S12P-R18P mutant.

FIG. 17 shows analytical size exclusion chromatography (aSEC) data of wild type and KS KO mutant versions of Adalimumab post-MSS purification. Percent of expected main peak is shown.

FIG. 18 shows analysis of samples of WT Adalimumab and the three CL mutants taken at the end of an accelerated stability test (40° C. for 2 weeks). Top panels shows a Coomassie-stained SDS-PAGE gel of non-reduced and reduced samples. Bottom panels show aSEC data for each respective sample is shown. Percent of expected main peak.

FIG. 19 shows DSC Analysis of F(ab)'2 derived from WT Adalimumab and three KS KO mutants.

FIG. 20 shows nano-DSF data and derived Tm are shown for WT Adalimumab and the three KS KO mutants.

FIG. 21 shows a Coomassie-stained SDS-PAGE gel of non-reduced and reduced samples following a three-step purification process. Identity of select high molecular weight bands is shown on left.

FIG. 22 shows a Coomassie-stained SDS-PAGE gel of non-reduced and reduced samples following a three-step purification process. Identity of select high molecular weight bands is shown on left.

FIG. 23A shows a chromatogram of MSS purification of a bispecific antibody with KS KO and ProL KO mutations as part of a three-step purification process. Chromatograms of the MSS step, the KS step and the ProL step are shown in FIG. 23B.

FIG. 24. shows a Coomassie-stained SDS-PAGE gel of samples from the trispecific CODV purification.

FIG. 25A provides a chromatogram for the adalimumab variant comprising His198Arg substituted light chains, wherein amino acid numbering is according to the EU Index.

FIG. 25B provides a chromatogram for the adalimumab variant comprising Gln199Trp substituted light chains, wherein amino acid numbering is according to the EU Index.

### DETAILED DESCRIPTION

### Overview

The invention provides multivalent binding proteins comprising four polypeptide chains, wherein a first heavy chain polypeptide and a first light chain polypeptide associate to form one or more antigen binding domains and a second heavy chain polypeptide and a second light chain polypeptide associate to bind one or more antigen binding domains. In some embodiments, the multivalent binding protein binds to more than one antigen. To reduce the presence of proteins with mispaired polypeptide chains formed during production of the multivalent binding proteins, amino acid substitutions are introduced into one light chain to essentially prevent it from binding to a Kappa Select chromatography material and amino acid substitutions are introduced into the other light chain polypeptide to essentially prevent it from binding to a Protein L chromatography material. Knob-into-hole and RF mutations (e.g., H435R and 436F mutations, wherein amino acid numbering is according to the EU Index) may also be included in Fc portions of the multivalent binding proteins to further reduce mispaired polypeptides in the multivalent binding protein preparations.

The invention also provides two-step and three-step chromatography methods for the purification of the multivalent binding proteins of the invention utilizing Kappa Select and Protein L chromatography with or without Protein A chromatography. Proteins comprising mispaired polypeptides are removed from preparations of the multivalent binding protein based on their lack of Kappa Select binding and/or protein L binding.

### General Definitions

As utilized in accordance with the present disclosure, the following terms, unless otherwise indicated, shall be understood to have the following meanings. Unless otherwise required by context, singular terms shall include pluralities and plural terms shall include the singular.

The term “polynucleotide” as used herein refers to single-stranded or double-stranded nucleic acid polymers of at least 10 nucleotides in length. In certain embodiments, the nucleotides comprising the polynucleotide can be ribonucleotides or deoxyribonucleotides or a modified form of either type of nucleotide. Such modifications include base modifications such as bromouridine, ribose modifications such as arabinoside and 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single-stranded and double-stranded forms of DNA.

An “isolated polynucleotide” is a polynucleotide of genomic, cDNA, or synthetic origin or some combination thereof, which: (1) is not associated with all or a portion of a polynucleotide in which the isolated polynucleotide is found in nature, (2) is linked to a polynucleotide to which it is not linked in nature, or (3) does not occur in nature as part of a larger sequence.

An “isolated polypeptide” is one that: (1) is free of at least some other polypeptides with which it would normally be found, (2) is essentially free of other polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a polypeptide with which the “isolated polypeptide” is associated in nature, (6) is operably associated (by covalent or noncovalent interaction) with a polypeptide with which it is not associated in nature, or (7) does not occur in nature. Such an isolated polypeptide can be encoded by genomic DNA, cDNA, mRNA or other RNA, of synthetic origin, or any combination thereof. Preferably, the isolated polypeptide is substantially free from polypeptides or other contaminants that are found in its natural environment that would interfere with its use (therapeutic, diagnostic, prophylactic, research or otherwise).

Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}), wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196: 901-17; Chothia et al., 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. Identification of predicted CDRs using the amino acid sequence is well known in the field, such as in Martin, A. C. “Protein sequence and structure analysis of antibody variable domains,” In Antibody Engineering, Vol. 2. Kontermann R., Dübel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG₂ are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG₂, IgG₃, IgA1, and IgGA2). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the V_{H} and C_{H1} domains of one heavy chain, wherein the V_{H}-C_{H1} heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a C_{H1} domain.

A F(ab′) fragment typically includes one light chain and a portion of one heavy chain that contains more of the constant region (between the C_{H1} and C_{H2} domains), such that an interchain disulfide bond can be formed between two heavy chains to form a F(ab′)₂ molecule.

The term “multivalent binding protein” as used herein refers to a non-naturally occurring (or recombinant or engineered) molecule that comprises more than one binding domain wherein the more than one binding domains bind to more than one target antigen. In some examples the binding protein comprises four polypeptide chains, typically a first heavy chain polypeptide and a first light chain polypeptide that associate to form at least one antigen binding domain and a second heavy chain polypeptide and a second light chain polypeptide that associate to form at least one antigen binding domain.

A “recombinant” molecule is one that has been prepared, expressed, created, or isolated by recombinant means.

One embodiment of the disclosure provides multivalent binding proteins having biological and immunological specificity to more than one antigen. Another embodiment of the disclosure provides nucleic acid molecules comprising nucleotide sequences encoding polypeptide chains that form such multivalent binding proteins. Another embodiment of the disclosure provides expression vectors comprising nucleic acid molecules comprising nucleotide sequences encoding polypeptide chains that form such multivalent binding proteins. Yet another embodiment of the disclosure provides host cells that express such multivalent binding proteins (i.e., comprising nucleic acid molecules or vectors encoding polypeptide chains that form such binding proteins).

The term “swapability” as used herein refers to the interchangeability of variable domains within the binding protein format and with retention of folding and ultimate binding affinity. “Full swapability” refers to the ability to swap the order of both V_{H1} and V_{H2} domains, and therefore the order of V_{L1} and V_{L2} domains, in the polypeptide chain of formula I or the polypeptide chain of formula II (i.e., to reverse the order) while maintaining full functionality of the binding protein as evidenced by the retention of binding affinity. Furthermore, it should be noted that the designations V_{H} and V_{L} refer only to the domain's location on a particular protein chain in the final format. For example, V_{H1} and V_{H2} could be derived from V_{L1} and V_{L2} domains in parent antibodies and placed into the V_{H1} and V_{H2} positions in the binding protein. Likewise, V_{L1} and V_{L2} could be derived from V_{H1} and V_{H2} domains in parent antibodies and placed in the V_{H1} and V_{H2} positions in the binding protein. Thus, the V_{H} and V_{L} designations refer to the present location and not the original location in a parent antibody. V_{H} and V_{L} domains are therefore “swappable.”

The term “antigen” or “target antigen” or “antigen target” as used herein refers to a molecule or a portion of a molecule that is capable of being bound by a binding protein, and additionally is capable of being used in an animal to produce antibodies capable of binding to an epitope of that antigen. A target antigen may have one or more epitopes. With respect to each target antigen recognized by a binding protein, the binding protein is capable of competing with an intact antibody that recognizes the target antigen.

The term “monospecific binding protein” refers to a binding protein that specifically binds to one antigen target.

The term “monovalent binding protein” refers to a binding protein that has one antigen binding site.

The term “bispecific binding protein” refers to a binding protein that specifically binds to two different antigen targets.

The term “bivalent binding protein” refers to a binding protein that has two binding sites.

The term “trispecific binding protein” refers to a binding protein that specifically binds to three different antigen targets.

The term “trivalent binding protein” refers to a binding protein that has three binding sites. In particular embodiments the trivalent binding protein can bind to one antigen target. In other embodiments, the trivalent binding protein can bind to two antigen targets. In other embodiments, the trivalent binding protein can bind to three antigen targets.

The term “tetraspecific binding protein” refers to a binding protein that specifically binds to four different antigen targets.

The term “tetravalent binding protein” refers to a binding protein that has four binding sites. In particular embodiments the tetravalent binding protein can bind to one antigen target. In other embodiments, the tetravalent binding protein can bind to two antigen targets. In other embodiments, the tetravalent binding protein can bind to three antigen targets. In other embodiments, the tetravalent binding protein can bind to four antigen targets.

An “isolated” binding protein is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes. In some embodiments, the binding protein will be purified:(1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding proteins include the binding protein in situ within recombinant cells since at least one component of the binding protein's natural environment will not be present.

The terms “substantially pure” or “substantially purified” as used herein refer to a compound or species that is the predominant species present (i.e., on a molar basis it is more abundant than any other individual species in the composition). In some embodiments, such terms are relative and do not necessarily mean absolute purity. In some embodiments, a substantially purified fraction is a composition wherein the species comprises at least about 50% (on a molar basis) of all macromolecular species present. In other embodiments, a substantially pure composition will comprise more than about 80%, 85%, 90%, 95%, or 99% of all macromolecular species present in the composition. In still other embodiments, the species is purified to essential homogeneity (contaminant species cannot be detected in the composition by conventional detection methods) wherein the composition consists essentially of a single macromolecular species. In still further embodiments, the species has been increased in purity, such that it exists in a form that is more pure than it exists in its natural environment and/or when initially synthesized and/or amplified under laboratory conditions.

A “neutralizing” binding protein as used herein refers to a molecule that is able to block or substantially reduce an effector function of a target antigen to which it binds. As used herein, “substantially reduce” means at least about 60%, preferably at least about 70%, more preferably at least about 75%, even more preferably at least about 80%, still more preferably at least about 85%, most preferably at least about 90% reduction of an effector function of the target antigen.

The term “epitope” includes any determinant, preferably a polypeptide determinant, capable of specifically binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics and/or specific charge characteristics. An epitope is a region of an antigen that is bound by an antibody or binding protein. In certain embodiments, a binding protein is said to specifically bind an antigen when it preferentially recognizes its target antigen in a complex mixture of proteins and/or macromolecules. In some embodiments, a binding protein is said to specifically bind an antigen when the equilibrium dissociation constant is ≤10⁻⁸ M, more preferably when the equilibrium dissociation constant is ≤10⁻⁹ M, and most preferably when the dissociation constant is ≤10⁻¹⁰ M.

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the BIAcore system (Pharmacia Biosensor; Piscataway, NJ). Surface plasmon analysis can also be performed by immobilizing the analyte (binding protein on a biosensor matrix) and presenting the ligand (target antigen). The term “K_{D},” as used herein refers to the dissociation constant of the interaction between a particular binding protein and a target antigen.

The term “specifically binds” as used herein refers to the ability of a binding protein or an antigen-binding fragment thereof to bind to an antigen containing an epitope with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to an epitope with an affinity that is at least two-fold greater than its affinity for a nonspecific antigen.

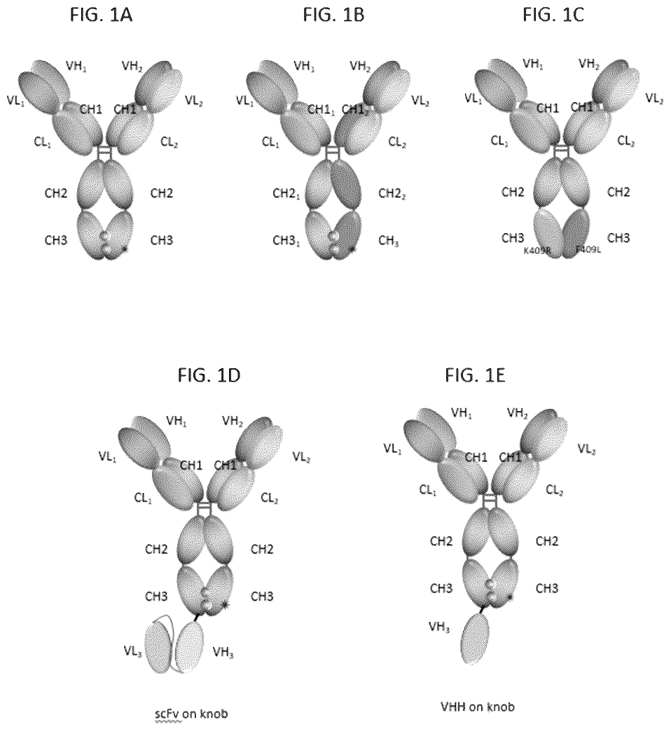

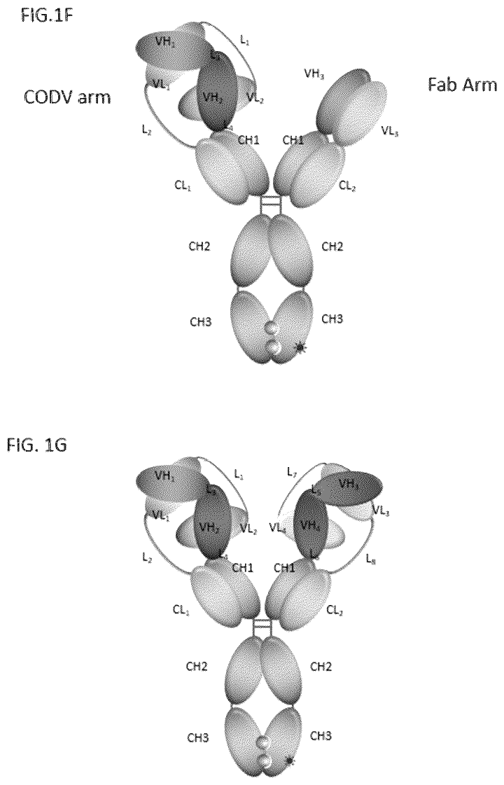

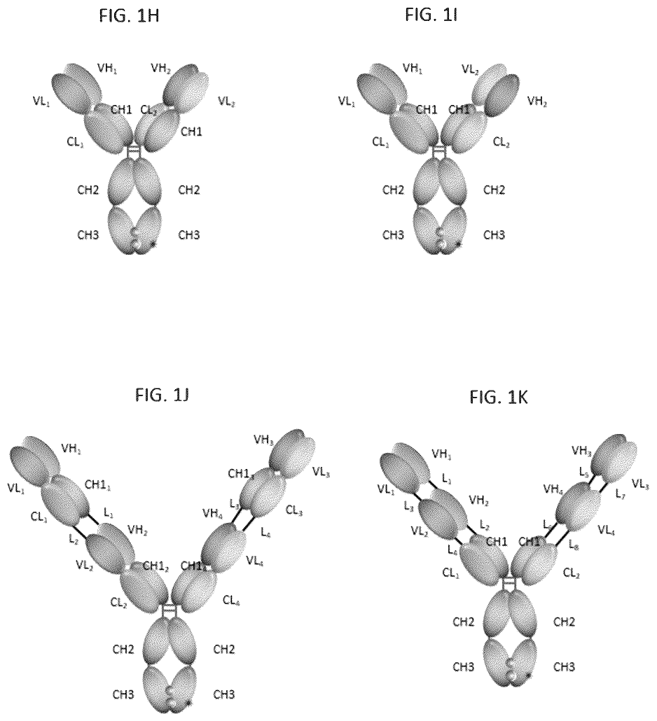

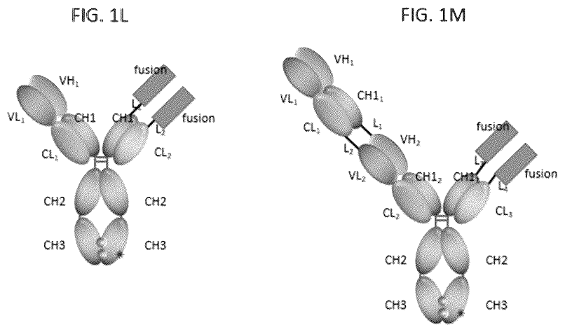

The term “linker” as used herein refers to one or more amino acid residues inserted between immunoglobulin domains to provide sufficient mobility for the domains of the light and heavy chains to fold into cross over dual variable region immunoglobulins. A linker is inserted at the transition between variable domains or between variable and constant domains, respectively, at the sequence level. The transition between domains can be identified because the approximate size of the immunoglobulin domains are well understood. The precise location of a domain transition can be determined by locating peptide stretches that do not form secondary structural elements such as beta-sheets or alpha-helices as demonstrated by experimental data or as can be assumed by techniques of modeling or secondary structure prediction. For example, with regard to the exemplary multivalent binding protein shown in FIG. 1F, the linker referred to as L₁, which is located on the light chain between the C-terminus of the VL₂ and the N-terminus of the VL₁ domain; and L₂, which is located on the light chain between the C-terminus of the VL₁ and the N-terminus of the C_{L} domain. The heavy chain linkers are known as L₃, which is located between the C-terminus of the VH₁ and the N-terminus of the VH₂ domain; and L₄, which is located between the C-terminus of the VH₂ and the N-terminus of the C_{H1} domain.

The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the disclosure is intended to include other forms of expression vectors, such as viral vectors (e.g., replication defective retroviruses, adenoviruses, and adeno-associated viruses), which serve equivalent functions.

The phrase “recombinant host cell” (or “host cell”) as used herein refers to a cell into which a recombinant expression vector has been introduced. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the binding proteins, including bacterial, yeast, baculoviral, and mammalian expression systems (as well as phage display expression systems). An example of a suitable bacterial expression vector is pUC19. To express a binding protein recombinantly, a host cell is transformed or transfected with one or more recombinant expression vectors carrying DNA fragments encoding the polypeptide chains of the binding protein such that the polypeptide chains are expressed in the host cell and, preferably, secreted into the medium in which the host cells are cultured, from which medium the binding protein can be recovered.

The term “transformation” as used herein refers to a change in a cell's genetic characteristics, and a cell has been transformed when it has been modified to contain a new DNA. For example, a cell is transformed where it is genetically modified from its native state. Following transformation, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “transfection” as used herein refers to the uptake of foreign or exogenous DNA by a cell, and a cell has been “transfected” when the exogenous DNA has been introduced inside the cell membrane. A number of transfection techniques are well known in the art. Such techniques can be used to introduce one or more exogenous DNA molecules into suitable host cells.

The term “naturally occurring” as used herein and applied to an object refers to the fact that the object can be found in nature and has not been manipulated by man. For example, a polynucleotide or polypeptide that is present in an organism (including viruses) that can be isolated from a source in nature and that has not been intentionally modified by man is naturally occurring. Similarly, “non-naturally occurring” as used herein refers to an object that is not found in nature or that has been structurally modified or synthesized by man.

As used herein, the twenty conventional amino acids and their abbreviations follow conventional usage. Stereoisomers (e.g., D-amino acids) of the twenty conventional amino acids; unnatural amino acids and analogs such as α-, α-disubstituted amino acids, N-alkyl amino acids, lactic acid, and other unconventional amino acids may also be suitable components for the polypeptide chains of the binding proteins. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:
(1) hydrophobic: Met, Ala, Val, Leu, Ile, Phe, Trp, Tyr, Pro;
(2) polar hydrophilic: Arg, Asn, Asp, Gln, Glu, His, Lys, Ser, Thr;
(3) aliphatic: Ala, Gly, Ile, Leu, Val, Pro;
(4) aliphatic hydrophobic: Ala, Ile, Leu, Val, Pro;
(5) neutral hydrophilic: Cys, Ser, Thr, Asn, Gln;
(6) acidic: Asp, Glu;
(7) basic: His, Lys, Arg;
(8) residues that influence chain orientation: Gly, Pro;
(9) aromatic: His, Trp, Tyr, Phe; and
(10) aromatic hydrophobic: Phe, Trp, Tyr.

Conservative amino acid substitutions may involve exchange of a member of one of these classes with another member of the same class. Non-conservative substitutions may involve the exchange of a member of one of these classes for a member from another class.

A skilled artisan will be able to determine suitable variants of the polypeptide chains of the multivalent binding proteins using well-known techniques. For example, one skilled in the art may identify suitable areas of a polypeptide chain that may be changed without destroying activity by targeting regions not believed to be important for activity. Alternatively, one skilled in the art can identify residues and portions of the molecules that are conserved among similar polypeptides. In addition, even areas that may be important for biological activity or for structure may be subject to conservative amino acid substitutions without destroying the biological activity or without adversely affecting the polypeptide structure.

The terms “pharmaceutical composition” or “therapeutic composition” as used herein refer to a compound or composition capable of inducing a desired therapeutic effect when properly administered to a patient.

The term “pharmaceutically acceptable carrier” or “physiologically acceptable carrier” as used herein refers to one or more formulation materials suitable for accomplishing or enhancing the delivery of a binding protein.

The terms “effective amount” and “therapeutically effective amount” when used in reference to a pharmaceutical composition comprising one or more binding proteins refer to an amount or dosage sufficient to produce a desired therapeutic result. More specifically, a therapeutically effective amount is an amount of a binding protein sufficient to inhibit, for some period of time, one or more of the clinically defined pathological processes associated with the condition being treated. The effective amount may vary depending on the specific binding protein that is being used, and also depends on a variety of factors and conditions related to the patient being treated and the severity of the disorder. For example, if the binding protein is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

One embodiment of the disclosure provides a pharmaceutical composition comprising a pharmaceutically acceptable carrier and a therapeutically effective amount of a binding protein.

All references cited herein, including patent applications, patent publications, and UniProtKB/Swiss-Prot Accession numbers are herein incorporated by reference in their entirety, as if each individual reference were specifically and individually indicated to be incorporated by reference.

Multivalent Binding Proteins with Reduced Binding to a KappaSelect and/or a Protein L Chromatography Material

### Exemplary Bivalent Binding Proteins

In some embodiments, provided is a multivalent binding protein (e.g., a bispecific antibody) comprising four polypeptide chains that form two antigen binding domains; wherein the four polypeptide chains comprise: a first heavy chain polypeptide comprising a structure represented by the formula:

VH₁-CH1 [I],
first light chain polypeptide chain comprising a structure represented by the formula:

VL₁-CL₁ [II],
second heavy chain polypeptide comprising a structure represented by the formula:

VH₂-CH1 [III],
and a second light chain polypeptide chain comprising a structure represented by the formula:

VL₂-CL₂ [IV];
wherein: VL₁ is a first immunoglobulin light chain variable domain; VL₂ is a second immunoglobulin light chain variable domain, wherein VL₂ is a κ1, κ3, or κ4 subtype light chain variable domain, CL₁ is a first immunoglobulin light chain constant domain, wherein CL₁ is a Cκ subtype light chain constant domain; CL₂ is a second immunoglobulin light chain constant domain; VH₁ is a first immunoglobulin heavy chain variable domain; VH₂ is a second immunoglobulin heavy chain variable domain; CH1 is an immunoglobulin heavy chain constant domain; wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and wherein VH₁ and VL₁ associate to form a first antigen binding domain and VH₂ and VL₂ associate to form a second antigen binding domain.

In some embodiments, provided is a binding protein (e.g., a multivalent binding protein) comprising four polypeptide chains that form two antigen binding domains; wherein the four polypeptide chains comprise: a first heavy chain polypeptide comprising a structure represented by the formula:

VH₁-CH1-CH2-CH3 [I],
a first light chain polypeptide chain comprising a structure represented by the formula:

VL₁-CL₁ [II],
a second heavy chain polypeptide comprising a structure represented by the formula:

VH₂-CH1-CH2-CH3 [III],
and a second light chain polypeptide chain comprising a structure represented by the formula:

VL₂-CL₂ [IV];
wherein: VL₁ is a first immunoglobulin light chain variable domain; VL₂ is a second immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; CH1, CH2 and CH3 are immunoglobulin heavy chain constant domains; wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and wherein VH₁ and VL₁ associate to form a first antigen binding domain and VH₂ and VL₂ associate to form a second antigen binding domain. An example of this embodiment is shown in FIG. 1A.

In some embodiments, the binding of the CL₁ of the multivalent binding protein (e.g., the bispecific antibody) to a KappaSelect chromatography material is reduced by about 90%, as compared to the binding of a CL₁ of a multivalent binding protein (e.g., bispecific antibody) without the one or more amino acid substitutions. In some embodiments, the binding of the VL₂ of the multivalent binding protein to a protein L chromatography material is reduced by about 90%, as compared to the binding of a VL₂ of a multivalent binding protein without the one or more amino acid substitutions. In some embodiments, the one or more amino acid substitutions in the CL₁ of the multivalent binding protein is at a position corresponding to 109, 110 or 199, wherein amino acid numbering is according to the EU index (see, e.g., Edelman et al., 1969, Proc Nat Acad Sci USA 63: 78-85). In some embodiments, the one or more amino acid substitutions in the CL₁ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the VL₂ of the multivalent binding protein is a substitution of a framework amino acid. In some embodiments, the one or more amino acid substitutions in the VL₂ of the multivalent binding protein is at a position corresponding to 12 or 18, wherein amino acid numbering is according to Kabat (see, e.g., Kabat et al., Sequences of Proteins of Immunological Interest, Fifth Edition, National Institute of Health, Bethesda, Md. (1991)). In some embodiments, the one or more amino acid substitutions in the VL₂ of the multivalent binding protein is a S12P substitution, a R18P substitution, a R18Q substitution, S12P-R18P substitutions, or S12P-R18Q substitutions, wherein amino acid numbering is according to Kabat.

In some embodiments, the CH3 domain of the first heavy chain polypeptide and/or the CH3 domain of the second heavy chain polypeptide of the multivalent binding protein is a human IgG1 or IgG4 CH3 domain. In some embodiments, the CH3 domain of the first heavy chain polypeptide comprises amino acid substitutions at positions corresponding to (e.g., such as relative to) positions 354 and 366 of human IgG1, wherein amino acid numbering is according to the EU Index. See, e.g., Spiess et al. (2013) JBC, 288(37): P26583-26593. In some embodiments, the amino acid substitutions are (or correspond to) S354C and T366W of human IgG1, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. In some embodiments, the CH3 domain of the first heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 354 and 366 of human IgG1, wherein numbering is according to the EU Index. In some embodiments, the amino acid substitutions are (or correspond to) S354C and T366W, wherein amino acid numbering is according to the EU Index.

In some embodiments, the CH3 domain of the second heavy chain polypeptide of the multivalent binding protein comprises (such as further comprises) one or more amino acid substitutions which reduces binding to protein A. In some embodiments, the CH3 domain of the first heavy chain polypeptide of the multivalent binding protein comprises (such as further comprises) one or more amino acid substitutions which reduces binding to protein A. In some embodiments, the one or more amino acid substitutions in the CH3 domain of the first or second heavy chain polypeptide which reduce binding to a Protein A chromatography material are amino acid substitutions at positions corresponding to (e.g., such as relative to) positions 435 and 436 of human IgG1, wherein numbering is according to the EU Index. In some embodiments, the amino acid substitutions are (or correspond to) H435R and Y436F wherein amino acid numbering is according to the EU Index.

In some embodiments, the CH1, CH2 and CH3 domains of the first heavy chain polypeptide of the multivalent binding protein are different from the CH1, CH2 and CH3 domains of the second heavy chain polypeptide. In some embodiments, the first heavy chain polypeptide is derived from a different species than the second heavy chain polypeptide. Additionally or alternatively, in some embodiments, the first light chain polypeptide is derived from a different species than the second light chain polypeptide. An example of this embodiment is shown in FIG. 1B. In some embodiments, the first heavy chain polypeptide and the first light chain polypeptide are derived from a mouse heavy chain immunoglobulin and a mouse light chain immunoglobulin, respectively, and the second heavy chain polypeptide and the second light chain polypeptides are derived from a rat heavy chain immunoglobulin and a rat light chain immunoglobulin, respectively. In some embodiments, the first heavy chain polypeptide and the first light chain polypeptide are derived from a rat heavy chain immunoglobulin and a rat light chain immunoglobulin, respectively, and the second heavy chain polypeptide and the second light chain polypeptides are derived from a mouse heavy chain immunoglobulin and a mouse light chain immunoglobulin, respectively.

In some embodiments, the first heavy chain polypeptide and the second heavy chain polypeptide of the multivalent binding protein each comprises an IgG4 CH3 domain. An example of this embodiment is shown in FIG. 1C. In some embodiments, the first heavy chain polypeptide comprises a K409R amino acid substitution and the second heavy chain polypeptide comprises a F405L amino acid substitution, wherein numbering is according to the EU index. (See FIG. 1C.) In some embodiments, the first heavy chain polypeptide comprises a F405L amino acid substitution and the second heavy chain polypeptide comprises a K409R amino acid substitution, wherein numbering is according to the EU index. In some embodiments, the multivalent binding protein is a bispecific antigen binding protein. In some embodiments, the first antigen binding domain and the second antigen binding domain bind different antigens. In some embodiments, the first antigen binding domain and the second antigen binding domain bind to different epitopes on the same antigen. In some embodiments, multivalent binding protein is a multispecific antibody or antigen binding fragment thereof.

### Exemplary Trivalent Binding Proteins

In some embodiments, the first heavy chain polypeptide chain of the multivalent binding protein comprises a structure represented by the formula:

VH₁-CH1-CH2-CH3-VH₃-L-VL₃ [Ia],
first light chain polypeptide chain comprising a structure represented by the formula:

VL₁-CL₁ [II],
the second heavy chain polypeptide comprising a structure represented by the formula:

VH₂-CH1-CH2-CH3 [IIIa],
and a second light chain polypeptide chain comprising a structure represented by the formula:

VL₂-CL₂ [IV];
wherein: VL₁ is a first immunoglobulin light chain variable domain; VL₂ is a second immunoglobulin light chain variable domain; VL₃ is a third immunoglobulin light chain variable domain; CL₁ is a first immunoglobulin light chain constant domain; CL₂ is a second immunoglobulin light chain constant domain; VH₁ is a first immunoglobulin heavy chain variable domain; VH₂ is a second immunoglobulin heavy chain variable domain; VH₃ is a third immunoglobulin heavy chain variable domain; CH1, CH2 and CH3 are immunoglobulin heavy chain constant domains; wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and wherein VH₁ and VL₁ associate to form a first antigen binding domain and VH₂ and VL₂ associate to form a second antigen binding domain, and VH₃ and VL₃ associate to form a third antigen binding domain, and wherein VH₃ and VL₃ are connected via amino acid linker L. In some embodiments, VL₃ does not bind protein L chromatography material. In some embodiments, VL₃ is a λ subtype immunoglobulin light chain variable domain or a λ2 immunoglobulin light chain variable domain. In some embodiments, L is 0 amino acids in length. In some embodiments, a linker of 0 amino acids in length indicates that the linker is absent from the binding protein. In some embodiments, L is at least one amino acid in length. An example of this embodiment is shown in FIG. 1D. In some embodiments, the first heavy chain polypeptide chain of the multivalent binding protein comprises a structure represented by the formula:

VH₁-CH1-CH2-CH3-VH₃ [Ib],
the second heavy chain polypeptide comprising a structure represented by the formula:

VH₂-CH1-CH2-CH3 [IIIb].

An example of this embodiment is shown in FIG. 1E. In some embodiments, the multivalent binding protein is bispecifc. In some embodiments, the multivalent binding protein is trispecific. In some embodiments, the first antigen binding domain, the second antigen binding domain, and the third antigen binding domains bind two or three different antigens. In some embodiments, the first antigen binding domain binds a first antigen, the second antigen binding domain binds a second antigen, and the third antigen binding domain binds a third antigen. In some embodiments, the first antigen binding domain and the second antigen binding domain bind a first antigen and the third antigen binding domain binds a second antigen. In some embodiments, multivalent binding protein is a multispecific antibody or antigen binding fragment thereof.

In some embodiments, the binding of the CL₁ of the multivalent binding protein (e.g., the trispecific antibody) to a KappaSelect chromatography material is reduced by about 90%, as compared to the binding of a CL₁ of a multivalent binding protein (e.g., bispecific antibody) without the one or more amino acid substitutions. In some embodiments, the binding of the VL₂ of the multivalent binding protein to a protein L chromatography material is reduced by about 90%, as compared to the binding of a VL₂ of a multivalent binding protein without the one or more amino acid substitutions. In some embodiments, the binding of the VL₃ of the multivalent binding protein to a protein L chromatography material is reduced by about 90%, as compared to the binding of a VL₃ of a multivalent binding protein without the one or more amino acid substitutions. In some embodiments, the one or more amino acid substitutions in the CL₁ of the multivalent binding protein is at a position corresponding to 109, 110 or 199, wherein numbering is according to the EU index (see, e.g., Edelman et al., 1969, Proc Natl Acad Sci USA 63: 78-85). In some embodiments, the one or more amino acid substitutions in the CL₁ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the CL₁ of the multivalent binding protein is at a position corresponding to 109, 198, 199, or 202, wherein numbering is according to the EU index (see, e.g., Edelman et al., 1969, Proc Natl Acad Sci USA 63: 78-85). In some embodiments, the one or more amino acid substitutions in the CL₁ is a H198R substitution, a Q199W substitution, or T109A-S202R substitutions, wherein amino acid numbering is according to the EU Index. In some embodiments, the one or more amino acid substitutions in the VL₂ of the multivalent binding protein is a substitution of a framework amino acid. In some embodiments, the one or more amino acid substitutions in the VL₂ of the multivalent binding protein is at a position corresponding to 12 or 18, wherein numbering is according to Kabat (see, e.g., Kabat et al., Sequences of Proteins of Immunological Interest, Fifth Edition, National Institute of Health, Bethesda, Md. (1991)). In some embodiments, the one or more amino acid substitutions in the VL₂ of the multivalent binding protein is a S12P substitution, a R18P substitution, a R18Q substitution, S12P-R18P substitutions, or S12P-R18Q substitutions, wherein numbering is according to Kabat. In some embodiments, the one or more amino acid substitutions in the VL₃ of the multivalent binding protein is a substitution of a framework amino acid. In some embodiments, the one or more amino acid substitutions in the VL₃ of the multivalent binding protein is at a position corresponding to 12 or 18, wherein numbering is according to Kabat (see, e.g., Kabat et al., Sequences of Proteins of Immunological Interest, Fifth Edition, National Institute of Health, Bethesda, Md. (1991)). In some embodiments, the one or more amino acid substitutions in the VL₃ of the multivalent binding protein is a S12P substitution, a R18P substitution, a R18Q substitution, S12P-R18P substitutions, or S12P-R18Q substitutions, wherein numbering is according to Kabat.

In some embodiments, the binding of the CL₂ of the multivalent binding protein (e.g., the trispecific antibody) to a KappaSelect chromatography material is reduced by about 90%, as compared to the binding of a CL₂ of a multivalent binding protein (e.g., bispecific antibody) without the one or more amino acid substitutions. In some embodiments, the binding of the VL₁ of the multivalent binding protein to a protein L chromatography material is reduced by about 90%, as compared to the binding of a VL₁ of a multivalent binding protein without the one or more amino acid substitutions. In some embodiments, the binding of the VL₃ of the multivalent binding protein to a protein L chromatography material is reduced by about 90%, as compared to the binding of a VL₃ of a multivalent binding protein without the one or more amino acid substitutions. In some embodiments, the one or more amino acid substitutions in the CL₂ of the multivalent binding protein is at a position corresponding to 109, 110 or 199, wherein numbering is according to the EU index (see, e.g., Edelman et al., 1969, Proc Natl Acad Sci USA 63: 78-85). In some embodiments, the one or more amino acid substitutions in the CL₂ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the CL₂ of the multivalent binding protein is at a position corresponding to 109, 198, 199, or 202, wherein numbering is according to the EU index (see, e.g., Edelman et al., 1969, Proc Natl Acad Sci USA 63: 78-85). In some embodiments, the one or more amino acid substitutions in the CL₂ is a H198R substitution, a Q199W substitution, or T109A-S202R substitutions, wherein amino acid numbering is according to the EU Index. In some embodiments, the one or more amino acid substitutions in the VL₁ of the multivalent binding protein is a substitution of a framework amino acid. In some embodiments, the one or more amino acid substitutions in the VL₁ of the multivalent binding protein is at a position corresponding to 12 or 18, wherein numbering is according to Kabat (see, e.g., Kabat et al., Sequences of Proteins of Immunological Interest, Fifth Edition, National Institute of Health, Bethesda, Md. (1991)). In some embodiments, the one or more amino acid substitutions in the VL₁ of the multivalent binding protein is a S12P substitution, a R18P substitution, a R18Q substitution, S12P-R18P substitutions, or S12P-R18Q substitutions, wherein numbering is according to Kabat. In some embodiments, the one or more amino acid substitutions in the VL₃ of the multivalent binding protein is a substitution of a framework amino acid. In some embodiments, the one or more amino acid substitutions in the VL₃ of the multivalent binding protein is at a position corresponding to 12 or 18, wherein numbering is according to Kabat (see, e.g., Kabat et al., Sequences of Proteins of Immunological Interest, Fifth Edition, National Institute of Health, Bethesda, Md. (1991)). In some embodiments, the one or more amino acid substitutions in the VL₃ of the multivalent binding protein is a S12P substitution, a R18P substitution, a R18Q substitution, S12P-R18P substitutions, or S12P-R18Q substitutions, wherein numbering is according to Kabat.

In some embodiments, the CH3 domain of the first heavy chain polypeptide and/or the CH3 domain of the second heavy chain polypeptide of the multivalent binding protein is a human IgG1 or IgG4 CH3 domain. In some embodiments, the CH3 domain of the first heavy chain polypeptide comprises amino acid substitutions at positions corresponding to (e.g., such as relative to) positions 354 and 366 of human IgG1, wherein amino acid numbering is according to the EU Index. See, e.g., Spiess et al. (2013) JBC, 288(37): P26583-26593. In some embodiments, the amino acid substitutions are (or correspond to) S354C and T366W of human IgG1, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. In some embodiments, the CH3 domain of the first heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 354 and 366 of human IgG1, wherein numbering is according to the EU Index. In some embodiments, the amino acid substitutions are (or correspond to) S354C and T366W, wherein amino acid numbering is according to the EU Index.

In some embodiments, the CH3 domain of the second heavy chain polypeptide of the multivalent binding protein comprises (such as further comprises) one or more amino acid substitutions which reduces binding to protein A. In some embodiments, the CH3 domain of the first heavy chain polypeptide of the multivalent binding protein comprises (such as further comprises) one or more amino acid substitutions which reduces binding to protein A. In some embodiments, the one or more amino acid substitutions in the CH3 domain of the first or second heavy chain polypeptide which reduce binding to a Protein A chromatography material are amino acid substitutions at positions corresponding to (e.g., such as relative to) positions 435 and 436 of human IgG1, wherein numbering is according to the EU Index. In some embodiments, the amino acid substitutions are (or correspond to) H435R and Y436F wherein amino acid numbering is according to the EU Index.

Exemplary CODV Multivalent Binding Proteins

In some embodiments, provided is a binding protein (e.g., a multivalent binding protein) comprising four polypeptide chains that form three antigen binding domains; wherein the four polypeptide chains comprise: a first heavy chain polypeptide comprising a structure represented by the formula:

VH₁-L₃-VH₂-L₄-CH1 [I],
a first light chain polypeptide chain comprising a structure represented by the formula:

VL₂-L₁-VL₁-L₂-CL₁ [II],
a second heavy chain polypeptide comprising a structure represented by the formula:

VH₃-CH1 [III],
and a second light chain polypeptide chain comprising a structure represented by the formula:

VL₃-CL₂ [IV]
wherein VL₁ is a first immunoglobulin light chain variable domain; VL₂ is a second immunoglobulin light chain variable domain; VL₃ is a third immunoglobulin light chain variable domain; VH₁ is a first immunoglobulin heavy chain variable domain; VH₂ is a second immunoglobulin heavy chain variable domain; VH₃ is a third immunoglobulin heavy chain variable domain; CL₁ is a first immunoglobulin light chain constant domain; CL₂ is a second immunoglobulin light chain constant domain; CH₁ is an immunoglobulin CH₁ heavy chain constant domain; and L₁, L₂, L₃ and L₄ are amino acid linkers; wherein the polypeptide of formula I and the polypeptide of formula II form a cross-over light chain-heavy chain pair; wherein VH₁ and VL₁ associate to form a first antigen binding domain, VH₂ and VL₂ associate to form a second antigen binding domain, and VH₃ and VL₃ associate to form a third antigen binding domain; and wherein:
a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype immunoglobulin light chain variable domain; or
d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain. In some embodiments, provided is a binding protein (e.g., a multivalent binding protein) comprising four polypeptide chains that form three antigen binding domains; wherein the four polypeptide chains comprise a first heavy chain polypeptide comprising a structure represented by the formula:

VH₁-L₃-VH₂-L₄-CH1-CH2-CH3 [Ia],
a first light chain polypeptide chain comprising a structure represented by the formula:

VL₂-L₁-VL₁-L₂-CL₁ [II],
a second heavy chain polypeptide comprising a structure represented by the formula:

VH₃-CH1-CH2-CH3 [IIIa],
and a second light chain polypeptide chain comprising a structure represented by the formula:

VL₃-CL₂ [IV]
wherein VL₁ is a first immunoglobulin light chain variable domain; VL₂ is a second immunoglobulin light chain variable domain; VL₃ is a third immunoglobulin light chain variable domain; VH₁ is a first immunoglobulin heavy chain variable domain; VH₂ is a second immunoglobulin heavy chain variable domain; VH₃ is a third immunoglobulin heavy chain variable domain; CL₁ is a first immunoglobulin light chain constant domain; CL₂ is a second immunoglobulin light chain constant domain; CH1 is an immunoglobulin CH1 heavy chain constant domain; CH2 is an immunoglobulin CH2 heavy chain constant domain; CH3 is an immunoglobulin CH3 heavy chain constant domain; and L₁, L₂, L₃ and L₄ are amino acid linkers; wherein the polypeptide of formula I and the polypeptide of formula II form a cross-over light chain-heavy chain pair; wherein VH₁ and VL₁ associate to form a first antigen binding domain, VH₂ and VL₂ associate to form a second antigen binding domain, and VH₃ and VL₃ associate to form a third antigen binding domain; and wherein:
a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₂ without the one or more amino acid substitutions;
b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype immunoglobulin light chain variable domain; or
d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain. An example of this embodiment is shown in FIG. 1F. In some embodiments, the binding protein is trispecific and capable of specifically binding three different antigen targets. In some embodiments, at least one of L₁, L₂, L₃ or L₄ is independently 0 amino acids in length. In some embodiments, a linker of 0 amino acids in length indicates that the linker is absent from the binding protein. In some embodiments, L₁, L₂, L₃ or L₄ are each independently at least one amino acid in length.

In some embodiments, the second heavy chain polypeptide of the binding protein (e.g., multivalent binding protein) comprises a structure represented by the formula:

VH₃-L₅-VH₄-L₆-CH1 [IIIb],
and the second light chain polypeptide chain of the binding protein (e.g., multivalent binding protein) comprises a structure represented by the formula:

VL₄-L₇-VL₃-L₈-CL₂ [IVa]
wherein VL₄ is a fourth immunoglobulin light chain variable domain, VH₄ is a fourth immunoglobulin heavy chain variable domain, L₅, L₆, L₇ and L₈ are amino acid linkers, wherein the polypeptide of formula IIIa and the polypeptide of formula IVa form a cross-over light chain-heavy chain pair, wherein VH₄ and VL₄ associate to form a fourth antigen binding domain; and wherein:
a) CL₁ comprises one or more amino acid substitutions which reduce binding to a KappaSelect chromatography material compared to a CL₁ without the one or more amino acid substitutions and VL₃ and VL₄ each comprise one or more amino acid substitutions which reduce binding to a protein L chromatography material compared to a VL₃ and VL₄ without the one or more amino acid substitutions;
b) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions, and wherein VL₄ is a λ subtype immunoglobulin light chain variable domain or a κ2 subtype light chain variable immunoglobulin domain;
c) CL₁ comprises one or more amino acid substitutions which reduce binding to a KappaSelect chromatography material compared to a CL₁ without the one or more amino acid substitutions and VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
d) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
e) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype immunoglobulin light chain variable domain; or
f) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain. In some embodiments, the second heavy chain polypeptide chain of the binding protein (e.g., multivalent binding protein) comprises a structure represented by the formula:

VH₃-L₅-VH₄-L₆-CH1-CH2-CH3 [IIIc],
wherein CH2 is an immunoglobulin CH2 heavy chain constant domain and CH3 is an immunoglobulin CH3 heavy chain constant domain. An example of this embodiment is shown in FIG. 1G. In some embodiments, the binding protein is tetraspecific and capable of specifically binding four different antigen targets. In some embodiments, at least one of L₁, L₂, L₃, L₄, L₅, L₆, L₇ or L₈ is independently 0 amino acids in length. In some embodiments, a linker of 0 amino acids in length indicates that the linker is absent from the binding protein. In some embodiments, L₁, L₂, L₃, L₄, L₅, L₆, L₇ or L₈ are each independently at least one amino acid in length.

In one embodiment, the binding protein of the disclosure is a trispecific and/or trivalent binding protein comprising four polypeptide chains that form three antigen binding sites that specifically bind one or more (e.g., three) different antigen targets or target proteins, wherein a first polypeptide chain comprises a structure represented by the formula:

VL₂-L₁-VL₁-L₂-CL₁ [I]
and a second polypeptide chain comprises a structure represented by the formula:

VH₁-L₃-VH₂-L₄-CH1-hinge-CH2-CH3 [II]
and a third polypeptide chain comprises a structure represented by the formula:

VH₃-CH1-hinge-CH₂-CH3 [III]
and a fourth polypeptide chain comprises a structure represented by the formula:

VL₃-CL₂ [IV]
wherein:
VL1 is a first immunoglobulin light chain variable domain;
VL2 is a second immunoglobulin light chain variable domain;
VL3 is a third immunoglobulin light chain variable domain;
VH1 is a first immunoglobulin heavy chain variable domain;
VH2 is a second immunoglobulin heavy chain variable domain;
VH3 is a third immunoglobulin heavy chain variable domain;
CL1 is a first immunoglobulin light chain constant domain;
CL2 is a second immunoglobulin light chain constant domain;
CH1 is an immunoglobulin CH1 heavy chain constant domain;
CH2 is an immunoglobulin CH2 heavy chain constant domain;
CH3 is an immunoglobulin CH3 heavy chain constant domain;
hinge is an immunoglobulin hinge region connecting the CH1 and CH2 domains; and L1, L2, L3 and L4 are amino acid linkers;
and wherein the polypeptide of formula I and the polypeptide of formula II form a cross-over light chain-heavy chain pair, wherein VH₁ and VL₁ associate to form a first antigen binding domain, VH₂ and VL₂ associate to form a second antigen binding domain, and VH₃ and VL₃ associate to form a third antigen binding domain; and wherein:
a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype immunoglobulin light chain variable domain; or
d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain.

In some embodiments, the first polypeptide chain and the second polypeptide chain have a cross-over orientation that forms two distinct antigen binding sites. In some embodiments, the VH₁ and VL₁ form a binding pair and form the first antigen binding site. In some embodiments, the VH₂ and VL₂ form a binding pair and form the second antigen binding site. In some embodiments, the third polypeptide and the fourth polypeptide form a third antigen binding site. In some embodiments, the VH₃ (e.g., the VH₃ of the third polypeptide) and VL₃ (e.g., the VL₃ of the fourth polypeptide) form a binding pair and form the third antigen binding site.

In some embodiments, the binding protein of the disclosure comprises three antigen binding sites that specifically bind one, two, or three antigen targets or target proteins (e.g., one antigen target, two different antigen targets, or three different antigen targets). In some embodiments, the binding protein binds three antigen targets. In some embodiments, the binding protein binds three different antigen targets. In some embodiments, two of the antigen binding sites bind the same antigen target. In those embodiments, the binding protein comprises the same binding domains twice, or different binding domains, and/or specifically binds different antigens or epitopes on the same antigen target. In some embodiments, three of the antigen binding sites bind the same antigen target. In those embodiments, the binding protein comprises the same binding domains three times, or different binding domains, and/or specifically binds different antigens or epitopes on the same antigen target.

In some embodiments, the binding of the CL₁ of the multivalent binding protein to a KappaSelect chromatography material is reduced by about 90% compared to the binding of a CL₁ of a multivalent binding protein without the one or more amino acid substitutions. In some embodiments, the binding of the VL₄ (if present) and/or the VL₃ to a protein L chromatography material is reduced by about 90% compared to the binding of a VL₄ (if present) and/or the VL₃ of a multivalent binding protein without the one or more amino acid substitutions. In some embodiments, the one or more amino acid substitutions in the CL₁ of the multivalent binding protein is at a position corresponding to 109, 110 or 199, wherein numbering is according to the EU index. In some embodiments, the one or more amino acid substitutions in the CL₁ is a T109A substitution, a V110D substitution, a Q199K substitution, T109A-V110D substitutions, or T109A-V 110D-Q199K substitutions, wherein amino acid numbering is according to the EU Index. In some embodiments, the one or more amino acid substitutions in the CL₁ of the multivalent binding protein is at a position corresponding to 109, 198, 199, or 202, wherein numbering is according to the EU index (see, e.g., Edelman et al., 1969, Proc Natl Acad Sci USA 63: 78-85). In some embodiments, the one or more amino acid substitutions in the CL₁ is a H198R substitution, a Q199W substitution, or T109A-S202R substitutions, wherein amino acid numbering is according to the EU Index. In some embodiments, the one or more amino acid substitutions in the VL₄ (if present) and/or the VL₃ is a substitution of a framework amino acid. In some embodiments, the one or more amino acid substitutions in the VL₄ (if present) and/or the VL₃ of the multivalent binding protein is at a position corresponding to 12 or 18, wherein numbering is according to Kabat. In some embodiments, the one or more amino acid substitutions in VL₄ (if present) and/or the VL₃ is a S12P substitution, a R18P substitution, a R18Q substitution, S12P-R18P substitutions, or S12P-R18Q substitutions, wherein amino acid numbering is according to Kabat.

In some embodiments, the binding of the CL₂ of the multivalent binding protein to a KappaSelect chromatography material is reduced by about 90% compared to the binding of a CL₂ of a multivalent binding protein without the one or more amino acid substitutions. In some embodiments, the binding of the VL₁ and/or the VL₂ to a protein L chromatography material is reduced by about 90% compared to the binding of a VL₁ and/or a VL₂ of a multivalent binding protein without the one or more amino acid substitutions. In some embodiments, the one or more amino acid substitutions in the CL₂ of the multivalent binding protein is at a position corresponding to 109, 110 or 199, wherein numbering is according to the EU index. In some embodiments, the one or more amino acid substitutions in the CL₂ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the CL₁ of the multivalent binding protein is at a position corresponding to 109, 198, 199, or 202, wherein numbering is according to the EU index (see, e.g., Edelman et al., 1969, Proc Natl Acad Sci USA 63: 78-85). In some embodiments, the one or more amino acid substitutions in the CL₁ is a H198R substitution, a Q199W substitution, or T109A-S202R substitutions, wherein amino acid numbering is according to the EU Index. In some embodiments, the one or more amino acid substitutions in the VL₁ and/or the VL₂ is a substitution of a framework amino acid. In some embodiments, the one or more amino acid substitutions in the VL₁ and/or the VL₂ of the multivalent binding protein is at a position corresponding to 12 or 18, wherein numbering is according to Kabat. In some embodiments, the one or more amino acid substitutions in VL₁ and/or VL₂ is a S12P substitution, a R18P substitution, a R18Q substitution, S12P-R18P substitutions, or S12P-R18Q substitutions, wherein amino acid numbering is according to Kabat.

In some embodiments, the CH3 domain of the first heavy chain polypeptide and/or the CH3 domain of the second heavy chain polypeptide of the multivalent binding protein is a human IgG1 or IgG4 CH3 domain. In some embodiments, the CH3 domain of the first heavy chain polypeptide comprises amino acid substitutions at positions corresponding to (e.g., such as relative to) positions 354 and 366 of human IgG1, wherein amino acid numbering is according to the EU Index. See, e.g., Spiess et al. (2013) JBC, 288(37): P26583-26593. In some embodiments, the amino acid substitutions are (or correspond to) S354C and T366W of human IgG1, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. In some embodiments, the CH3 domain of the first heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 354 and 366 of human IgG1, wherein numbering is according to the EU Index. In some embodiments, the amino acid substitutions are (or correspond to) S354C and T366W, wherein amino acid numbering is according to the EU Index.

In some embodiments, the CH3 domain of the second heavy chain polypeptide of the multivalent binding protein comprises (such as further comprises) one or more amino acid substitutions which reduces binding to protein A. In some embodiments, the CH3 domain of the first heavy chain polypeptide of the multivalent binding protein comprises (such as further comprises) one or more amino acid substitutions which reduces binding to protein A. In some embodiments, the one or more amino acid substitutions in the CH3 domain of the first or second heavy chain polypeptide which reduce binding to a Protein A chromatography material are amino acid substitutions at positions corresponding to (e.g., such as relative to) positions 435 and 436 of human IgG1, wherein numbering is according to the EU Index. In some embodiments, the amino acid substitutions are (or correspond to) H435R and Y436F wherein amino acid numbering is according to the EU Index.

In some embodiments, multivalent binding protein is a multispecific antibody or antigen binding fragment thereof.

Exemplary Cross-Mab Bivalent Binding Proteins

In some embodiments, the binding protein is a multivalent binding protein comprising four polypeptide chains that form two antigen binding domains; wherein the four polypeptide chains comprise a first heavy chain polypeptide comprising a structure represented by the formula:

VH₁-CH1 [I],
a first light chain polypeptide chain comprising a structure represented by the formula:

VL₁-CL₁ [II],
a second heavy chain polypeptide comprising a structure represented by the formula:

VH₂-CL₂ [III],
and a second light chain polypeptide chain comprising a structure represented by the formula:

VL₂-CH1 [IV];
wherein VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, VH₁ is a first immunoglobulin heavy chain variable domain, VH₂ is a second immunoglobulin heavy chain variable domain, CH₁ is an immunoglobulin heavy chain constant domain, and wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments, provided is a binding protein (e.g., a multivalent binding protein) comprising four polypeptide chains that form two antigen binding domains; wherein the four polypeptide chains comprise:
a first heavy chain polypeptide comprising a structure represented by the formula:

VH₁-CH1-CH2-CH3 [I],
a first light chain polypeptide chain comprising a structure represented by the formula:

VL₁-CL₁ [II],
a second heavy chain polypeptide comprising a structure represented by the formula:

VH₂-CL₂-CH2-CH3 [III],
and a second light chain polypeptide chain comprising a structure represented by the formula:

VL₂-CH1 [IV];
wherein VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, VH₁ is a first immunoglobulin heavy chain variable domain, VH₂ is a second immunoglobulin heavy chain variable domain. CH₁ is an immunoglobulin CH₁ heavy chain constant domain; CH₂ is an immunoglobulin CH₂ heavy chain constant domain; and CH3 is an immunoglobulin CH3 heavy chain constant domain; wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain. An example of this embodiment is shown in FIG. 1H.

In some embodiments, the binding protein is a multivalent binding protein comprising four polypeptide chains that form two antigen binding domains; wherein the four polypeptide chains comprise:
a first heavy chain polypeptide comprising a structure represented by the formula:

VH₁-CH1 [I],
a first light chain polypeptide chain comprising a structure represented by the formula:

VL₁-CL₁ [II],
a second heavy chain polypeptide comprising a structure represented by the formula:

VL₂-CH1 [III],
and a second light chain polypeptide chain comprising a structure represented by the formula:

VH₂-CL₂ [IV];
wherein VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, VH₁ is a first immunoglobulin heavy chain variable domain, VH₂ is a second immunoglobulin heavy chain variable domain, and CH₁ is an immunoglobulin CH₁ heavy chain constant domain; wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

In some embodiments, provided is a binding protein (e.g., a multivalent binding protein) comprising four polypeptide chains that form two antigen binding domains; wherein the four polypeptide chains comprise:
a first heavy chain polypeptide comprising a structure represented by the formula:

VH₁-CH1-CH2-CH3 [I],
a first light chain polypeptide chain comprising a structure represented by the formula:

VL₁-CL₁ [II],
a second heavy chain polypeptide comprising a structure represented by the formula:

VL₂-CH1-CH2-CH3 [III],
and a second light chain polypeptide chain comprising a structure represented by the formula:

VH₂-CL₂ [IV];
wherein VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain; VH₁ is a first immunoglobulin heavy chain variable domain, VH₂ is a second immunoglobulin heavy chain variable domain, CH1, CH2 and CH3 are immunoglobulin heavy chain constant domains; wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain. An example of this embodiment is show in FIG. 1I.

In some embodiments, the binding of the CL₁ or the CL₂ of the multivalent binding protein to a KappaSelect chromatography material is reduced by about 90%, as compared to the binding of a CL₁ or a CL₂ of a multivalent binding protein without the one or more amino acid substitutions. In some embodiments, the binding of the VL₁ or the VL₂ of the multivalent binding protein to a protein L chromatography material is reduced by about 90%, as compared to the binding of a VL₁ or a VL₂ of a multivalent binding protein without the one or more amino acid substitutions. In some embodiments, the one or more amino acid substitutions in the CL₁ or the CL₂ of the multivalent binding protein is at a position corresponding to 109, 110 or 199, wherein numbering is according to the EU index. In some embodiments, the one or more amino acid substitutions in the CL₁ or the CL₂ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the CL₁ or the CL₂ of the multivalent binding protein is at a position corresponding to 109, 198, 199, or 202, wherein numbering is according to the EU Index. In some embodiments, the one or more amino acid substitutions in the CL₁ or the CL₂ is a H198R substitution, a Q199W substitution, or T 109A-S202R substitutions, wherein amino acid numbering is according to the EU Index. In some embodiments, the one or more amino acid substitutions in the VL₃ or the VL₄ of the multivalent binding protein is a substitution of a framework amino acid. In some embodiments, the one or more amino acid substitutions in VL₃ or VL₄ is at a position corresponding to 12 or 18, wherein numbering is according to Kabat. In some embodiments, the one or more amino acid substitutions in the VL₃ or the VL₄ of the multivalent binding protein is a S12P substitution, a R18P substitution, a R18Q substitution, S12P-R18P substitutions, or S12P-R18Q substitutions, wherein numbering is according to Kabat.

In some embodiments, the CH3 domain of the first heavy chain polypeptide and/or the CH3 domain of the second heavy chain polypeptide of the multivalent binding protein is a human IgG1 or IgG4 CH3 domain. In some embodiments, the CH3 domain of the first heavy chain polypeptide comprises amino acid substitutions at positions corresponding to (e.g., such as relative to) positions 354 and 366 of human IgG1, wherein amino acid numbering is according to the EU Index. See, e.g., Spiess et al. (2013) JBC, 288(37): P26583-26593. In some embodiments, the amino acid substitutions are (or correspond to) S354C and T366W of human IgG1, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. In some embodiments, the CH3 domain of the first heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 354 and 366 of human IgG1, wherein numbering is according to the EU Index. In some embodiments, the amino acid substitutions are (or correspond to) S354C and T366W, wherein amino acid numbering is according to the EU Index.

In some embodiments, the CH3 domain of the second heavy chain polypeptide of the multivalent binding protein comprises (such as further comprises) one or more amino acid substitutions which reduces binding to protein A. In some embodiments, the CH3 domain of the first heavy chain polypeptide of the multivalent binding protein comprises (such as further comprises) one or more amino acid substitutions which reduces binding to protein A. In some embodiments, the one or more amino acid substitutions in the CH3 domain of the first or second heavy chain polypeptide which reduce binding to a Protein A chromatography material are amino acid substitutions at positions corresponding to (e.g., such as relative to) positions 435 and 436 of human IgG1, wherein numbering is according to the EU Index. In some embodiments, the amino acid substitutions are (or correspond to) H435R and Y436F wherein amino acid numbering is according to the EU Index.

In some embodiments, the multivalent binding protein is a bispecific antigen binding protein. In some embodiments, the first antigen binding domain and the second antigen binding of the multivalent binding protein domain bind different antigens. In some embodiments, multivalent binding protein is a multispecific antibody or antigen binding fragment thereof.

Exemplary Tandem Fab Binding Proteins

In some embodiments, provided is a binding protein (e.g., a multivalent binding protein) comprising four polypeptide chains that form four antigen binding domains, wherein the four polypeptide chains comprise:
a first heavy chain polypeptide comprising a structure represented by the formula:

VH₁-CH1₁-L₁-VH₂-CH1₂ [I],
a first light chain polypeptide comprising a structure represented by the formula:

VL₁-CL₁-L₂-VL₂-CL₂ [II],
a second heavy chain polypeptide comprising a structure represented by the formula:

VH₃-CH1₃-L₃-VH₄-CH1₄ [III],
a second light chain polypeptide comprising a structure represented by the formula:

VL₃-CL₃-L₄-VL₄-CL₄ [IV]
wherein VL₁ is a first immunoglobulin light chain variable domain, VL₂ is a second immunoglobulin light chain variable domain, VL₃ is a third immunoglobulin light chain variable domain, VL₄ is a fourth immunoglobulin light chain variable domain, CL₁ is a first immunoglobulin light chain constant domain, CL₂ is a second immunoglobulin light chain constant domain, CL₃ is a third immunoglobulin light chain constant domain, CL₄ is a fourth immunoglobulin light chain constant domain, VH₁ is a first immunoglobulin heavy chain variable domain, VH₂ is a second immunoglobulin heavy chain variable domain, VH₃ is a third immunoglobulin heavy chain variable domain, VH₄ is a fourth immunoglobulin heavy chain variable domain, CH1₁ is a first immunoglobulin heavy chain constant domain, CH1₂ is a second immunoglobulin heavy chain constant domain, CH1₃ is a third immunoglobulin heavy chain constant domain, CH1₄ is a fourth immunoglobulin heavy chain constant domain, and L₁, L₂, L₃ and L₄ are amino acid linkers; wherein:
a) CL₁ and CL₂ each comprise one or more amino acid substitutions which reduce binding to a KappaSelect chromatography material compared to a CL₁ and CL₂ without the one or more amino acid substitutions and VL₃ and VL₄ each comprise one or more amino acid substitutions which reduce binding to a protein L chromatography material compared to a VL₃ and VL₄ without the one or more amino acid substitutions;
b) CL₁ and CL₂ each comprise one or more amino acid substitutions which reduce binding to a KappaSelect chromatography material compared to a CL₁ and CL₂ without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype light chain variable immunoglobulin domain;
c) CL₁ and CL₂ each comprises one or more amino acid substitutions which reduce binding to a KappaSelect chromatography material compared to a CL₁ and CL₂ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a λ2 subtype immunoglobulin light chain variable domain;
d) CL₁ comprises one or more amino acid substitutions which reduce binding to a KappaSelect chromatography material compared to a CL₁ without the one or more amino acid substitutions, CL₂ is a λ subtype immunoglobulin light chain constant domain, and VL₃ and VL₄ each comprise one or more amino acid substitutions which reduce binding to a protein L chromatography material compared to a VL₃ and VL₄ without the one or more amino acid substitutions;
e) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
f) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
g) CL₂ comprises one or more amino acid substitutions which reduce binding to a KappaSelect chromatography material compared to a CL₂ without the one or more amino acid substitutions, CL₁ is a λ subtype immunoglobulin light chain constant domain, and VL₃ and VL₄ each comprise one or more amino acid substitutions which reduce binding to a protein L chromatography material compared to a VL₃ and VL₄ without the one or more amino acid substitutions;
h) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
i) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain. In some embodiments, at least one of L₁, L₂, L₃ or L₄ is independently 0 amino acids in length. In some embodiments, a linker of 0 amino acids in length indicates that the linker is absent from the binding protein. In some embodiments, L₂ and/or L₄ is absent. In some embodiments, L₁, L₂, L₃ or L₄ are each independently at least one amino acid in length.

In some embodiments, provided is a binding protein (e.g., a multivalent binding protein) comprising four polypeptide chains that form four antigen binding domains; wherein the four polypeptide chains comprise:
a first heavy chain polypeptide comprising a structure represented by the formula:

VH₁-L₁-VH₂-L₂-CH1₁ [I],
a first light chain polypeptide comprising a structure represented by the formula:

VL₁-L₃-VL₂-L₄-CL₁ [II],
a second heavy chain polypeptide comprising a structure represented by the formula:

VH₃-L₅-VH₄-L₆-CH1₂ [III],
a second light chain polypeptide comprising a structure represented by the formula:

VL₃-L₇-VL₄-L₈-CL₂ [IV]
wherein VL₁ is a first immunoglobulin light chain variable domain, VL₂ is a second immunoglobulin light chain variable domain, VL₃ is a third immunoglobulin light chain variable domain, VL₄ is a fourth immunoglobulin light chain variable domain, CL₁ is a first immunoglobulin light chain constant domain, CL₂ is a second immunoglobulin light chain constant domain, VH₁ is a first immunoglobulin heavy chain variable domain, VH₂ is a second immunoglobulin heavy chain variable domain, VH₃ is a third immunoglobulin heavy chain variable domain, VH₄ is a fourth immunoglobulin heavy chain variable domain, CH1₁ is a first immunoglobulin CH₁ heavy chain constant domain, CH1₂ is a second immunoglobulin CH1 heavy chain constant domain, and L₁, L₂, L₃, L₄ L₅, L₆, L₇ and L₈ are amino acid linkers; wherein:
a) CL₁ comprises one or more amino acid substitutions which reduce binding to a KappaSelect chromatography material compared to a CL₁ without the one or more amino acid substitutions and VL₃ and VL₄ each comprise one or more amino acid substitutions which reduce binding to a protein L chromatography material compared to a VL₃ and VL₄ without the one or more amino acid substitutions;
b) CL₁ comprise one or more amino acid substitutions which reduce binding to a KappaSelect chromatography material compared to a C_{L} without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
c) CL₁ comprises one or more amino acid substitutions which reduce binding to a KappaSelect chromatography material compared to a CL₁ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
d) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
e) CL₂ comprise one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin light chain variable domain;
f) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain;
and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain. In some embodiments, at least one of L₁, L₂, L₃, L₄, L₅, L₆, L₇, or L₈ is independently 0 amino acids in length. In some embodiments, a linker of 0 amino acids in length indicates that the linker is absent from the binding protein. In some embodiments, wherein at least one of L₃, L₄, L₇, or L₈ is absent. In some embodiments, L₁, L₂, L₃, L₄, L₅, L₆, L₇, or L₈ are each independently at least one amino acid in length. In some embodiments, the binding of the CL₁ or the CL₂ of the multivalent binding protein to a KappaSelect chromatography material is reduced by about 90%, as compared the binding of a CL₁ or a CL₂ of a multispecific binding protein without the one or more amino acid substitutions. In some embodiments, the binding of the VL₃ and/or the VL₄ of the multivalent binding protein to a protein L chromatography material is reduced by about 90%, as compared to the binding of a VL₃ and/or a VL₄ of a multivalent binding protein without the one or more amino acid substitutions. In some embodiments, the one or more amino acid substitutions in the CL₁ or the CL₂ of the multivalent binding protein is at a position corresponding to 109, 110 or 199, wherein numbering is according to the EU index. In some embodiments, the one or more amino acid substitutions in the CL₁ or the CL₂ is a T109A substitution, a V110D substitution, a Q199K substitution, T109A-V110D substitutions, or T109A-V110D-Q199K substitutions, wherein amino acid numbering is according to the EU Index. In some embodiments, the one or more amino acid substitutions in the CL₁ of the multivalent binding protein is at a position corresponding to 109, 198, 199, or 202, wherein numbering is according the EU Index. In some embodiments, the one or more amino acid substitutions in the CL₁ is a H198R substitution, a Q199W substitution, or T109A-S202R substitutions, wherein amino acid numbering is according to the EU Index. In some embodiments, the one or more amino acid substitutions in the VL₃ and/or the VL₄ of the multivalent binding protein is a substitution of a framework amino acid. In some embodiments, the one or more amino acid substitutions in the VL₃ and/or the VL₄ of the multivalent binding protein is at a position corresponding to 12 or 18, wherein numbering is according to Kabat. In some embodiments, the one or more amino acid substitutions in the VL₃ or the VL₄ is a S12P substitution, a R18P substitution, a R18Q substitution, S12P-R18P substitutions, or S12P-R18Q substitutions, wherein numbering is according to Kabat.

In some embodiments, the first heavy chain polypeptide of the multivalent binding protein comprises a structure represented by the formula:

VH₁-CH1₁-L₁-VH₂-CH1₂-CH2-CH3 [Ia],
and the second heavy chain polypeptide comprises a structure represented by the formula:

VH₃-CH1₃-L₃-VH₄-CH1₄-CH2-CH3 [IIIa].
An example of this embodiment is shown in FIG. 1J.

In some embodiments, the first heavy chain polypeptide of the multivalent binding peptide comprises a structure represented by the formula:

VH₁-L₁-VH₂-L₂-CH1₁-CH2-CH3 [Ia],
and the second heavy chain polypeptide comprises a structure represented by the formula:

VH₃-L₅-VH₄-L₆-CH1₂-CH2-CH3 [IIIa].
An example of this embodiment is shown in FIG. 1K.

In some embodiments, the CH3 domain of the first heavy chain polypeptide and/or the CH3 domain of the second heavy chain polypeptide of the multivalent binding protein is a human IgG1 or IgG4 CH3 domain. In some embodiments, the CH3 domain of the first heavy chain polypeptide comprises amino acid substitutions at positions corresponding to (e.g., such as relative to) positions 354 and 366 of human IgG1, wherein amino acid numbering is according to the EU Index. See, e.g., Spiess et al. (2013) JBC, 288(37): P26583-26593. In some embodiments, the amino acid substitutions are (or correspond to) S354C and T366W of human IgG1, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. In some embodiments, the CH3 domain of the first heavy chain polypeptide comprises amino acid substitutions at positions corresponding to (e.g., such as relative to) positions 349, 366, 368, 407, 435, and 436 of human IgG1, wherein numbering is according to the EU Index. In some embodiments, the amino acid substitutions are (or correspond to) Y349C, T366S, L368A, and Y407V, wherein amino acid numbering is according to the EU Index. Additionally or alternatively, in some embodiments, the CH3 domain of the second heavy chain polypeptide comprises amino acid substitutions at positions corresponding to (e.g., such as relative to) positions 354 and 366 of human IgG1, wherein numbering is according to the EU Index. In some embodiments, the amino acid substitutions are (or correspond to) S354C and T366W, wherein amino acid numbering is according to the EU Index.

In some embodiments, the CH3 domain of the second heavy chain polypeptide of the multivalent binding protein comprises (such as further comprises) one or more amino acid substitutions which reduces binding to protein A. In some embodiments, the CH3 domain of the first heavy chain polypeptide of the multivalent binding protein comprises (such as further comprises) one or more amino acid substitutions which reduces binding to protein A. In some embodiments, the one or more amino acid substitutions in the CH3 domain of the first or second heavy chain polypeptide which reduce binding to a Protein A chromatography material are amino acid substitutions at positions corresponding to (e.g., such as relative to) positions 435 and 436 of human IgG1, wherein numbering is according to the EU Index. In some embodiments, the amino acid substitutions are (or correspond to) H435R and Y436F wherein amino acid numbering is according to the EU Index.

In some embodiments, the binding protein is tetraspecific and capable of specifically binding four different antigen targets.

In some embodiments, multivalent binding protein is a multispecific antibody or antigen binding fragment thereof.

Exemplary Multivalent Binding Proteins that Comprise a Fusion Protein

In some embodiments, provided is a binding protein (e.g., a multivalent binding protein) comprising four polypeptide chains that form an antigen binding domain and a target binding domain; wherein the four polypeptide chains comprise:
a first polypeptide chain (e.g., a first heavy chain polypeptide) that comprises a structure represented by the formula:

VH₁-CH1₁ [I],
a second polypeptide chain (e.g., a first light chain polypeptide) that comprises a structure represented by the formula:

VL₁-CL₁ [II],
a third polypeptide (e.g., a second heavy chain polypeptide) that comprises a structure represented by the formula:

fusion polypeptide-L₁-CH1₂ [III] or

L₁-CH1₂ [IIIa]
and a fourth polypeptide (e.g., a second light chain polypeptide) that comprises a structure represented by the formula:

fusion polypeptide-L₂-CL₂ [IV] or

L₂-CL₂ [IVa]
wherein VL₁ is a first immunoglobulin light chain variable domain, CL₁ is a first immunoglobulin light chain constant domain, CL₂ is a second immunoglobulin light chain constant domain, VH₁ is a first immunoglobulin heavy chain variable domain, CH1₁ is a first CH₁ immunoglobulin heavy chain constant domain, CH1₂ is a second CH₁ immunoglobulin heavy chain constant domain; and L₁ and L₂ are amino acid linkers; wherein CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions; and wherein VL₁ and VH₁ form an antigen binding domain. In some embodiments, the binding protein (e.g., a multivalent binding protein) comprises a third polypeptide comprising [III] and a fourth polypeptide comprising formula [IV]. In some embodiments, the binding protein (e.g., a multivalent binding protein) comprises a third polypeptide comprising [IIIa] and a fourth polypeptide comprising formula [IV]. In some embodiments, the binding protein (e.g., a multivalent binding protein) comprises a third polypeptide comprising [III] and a fourth polypeptide comprising formula [IVa]. In some embodiments, wherein the binding protein (e.g., a multivalent binding protein) comprises a third polypeptide comprising [III] and a fourth polypeptide comprising formula [IV], each fusion polypeptide binds a target antigen. In some embodiments, the target antigens are the same. In some embodiments, the target antigens are different. In some embodiments, wherein the binding protein (e.g., a multivalent binding protein) comprises a third polypeptide comprising [III] and a fourth polypeptide comprising formula [IV], the fusion polypeptides dimerize and bind a single target. An example of the embodiments described above is shown in FIG. 1L. In some embodiments, L₁ and/or L₂ are independently 0 amino acids in length. In some embodiments, a linker of 0 amino acids in length indicates that the linker is absent from the binding protein. In some embodiments, L₁ and/or L₂ are independently at least one amino acid in length.

In some embodiments, the binding of the CL₂ of the multivalent binding protein to KappaSelect chromatography material is reduced by about 90%, as compared to the binding of a CL₂ of a multivalent binding protein without the one or more amino acid substitutions. In some embodiments, the binding of the VL₁ to a protein L chromatography material is reduced by about 90%, as compared to the binding of a VL₁ without the one or more amino acid substitutions. In some embodiments, the one or more amino acid substitutions in the CL₂ of the multivalent binding protein is at a position corresponding to 109, 110 or 199, wherein numbering is according to the EU index. In some embodiments, the one or more amino acid substitutions in of CL₂ is a T109A substitution, a V110D substitution, a Q199K substitution, T109A-V110D substitutions, or T109A-V 110D-Q199K substitutions, wherein amino acid numbering is according to the EU Index. In some embodiments, the one or more amino acid substitutions in the CL₂ of the multivalent binding protein is at a position corresponding to 109, 198, 199, or 202, wherein numbering is according to the EU Index. In some embodiments, the one or more amino acid substitutions in the CL₂ is a H198R substitution, a Q199W substitution, or T109A-S202R substitutions, wherein amino acid numbering is according to the EU Index. In some embodiments, the one or more amino acid substitutions in the VL₁ of the multivalent binding protein is a substitution of a framework amino acid. In some embodiments, the one or more amino acid substitutions in VL₁ is at a position corresponding to 12 or 18, wherein numbering is according to Kabat. In some embodiments, the one or more amino acid substitutions in VL₁ is a S12P substitution, a R18P substitution, a R18Q substitution, S12P-R18P substitutions, or S12P-R18Q substitutions, wherein numbering is according to Kabat.

In some embodiments, provided is a binding protein (e.g., a multivalent binding protein) comprising four polypeptide chains that form two antigen binding domains and a target binding domain; wherein the four polypeptide chains comprise:
a first polypeptide chain (e.g., a first heavy chain polypeptide) that comprises a structure represented by the formula:

VH₁-CH1₁-L₁-VH₂-CH1₂ [I],
a second polypeptide chain (e.g., a first light chain polypeptide) that comprises a structure represented by the formula:

VL₁-CL₁-L₂-VL₂-CL₂ [II],
a third polypeptide chain (e.g., a second heavy chain polypeptide) that comprises a structure represented by the formula:

fusion polypeptide-L₃-CH1₃ [III],
and a fourth polypeptide chain (e.g., a second light chain polypeptide) that comprises a structure represented by the formula:

CL₃ [IVa]
wherein: VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, CL₃ is a third immunoglobulin light chain constant domain, VH₁ is a first immunoglobulin heavy chain variable domain, VH₂ is a second immunoglobulin heavy chain variable domain, CH1₁ is a first immunoglobulin heavy chain constant domain, CH1₂ is a second immunoglobulin heavy chain constant domain, CH1₃ is a third immunoglobulin heavy chain constant domain, and L₁, L₂, and L₃ are amino acid linkers; wherein:
a) CL₃ comprises one or more amino acid substitutions which reduce binding to a KappaSelect chromatography material compared to a CL₃ without the one or more amino acid substitutions, VL₁ and VL₂ each comprise one or more amino acid substitutions which reduce binding to a protein L chromatography material compared to a VL₁ and VL₂ without the one or more amino acid substitutions,
b) CL₃ comprises one or more amino acid substitutions which reduce binding to a KappaSelect chromatography material compared to a CL₃ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin light chain variable domain; or
c) CL₃ comprises one or more amino acid substitutions which reduce binding to a KappaSelect chromatography material compared to a CL₃ without the one or more amino acid substitutions, VL₂ comprises one or more amino acid substitutions which reduce binding to a protein L chromatography material compared to a VL₂ without the one or more amino acid substitutions, and VL₁ is a λ subtype immunoglobulin light chain variable domain or a κ2 subtype immunoglobulin light chain variable domain; and
wherein VL₁ and VH₁ form a first antigen binding domain and VL₂ and VH₂ form a second antigen binding domain.

In some embodiments, provided is a binding protein (e.g., a multivalent binding protein) comprising four polypeptide chains that form two antigen binding domains and a target binding domain; wherein the four polypeptide chains comprise:
a first polypeptide chain (e.g., a first heavy chain polypeptide) that comprises a structure represented by the formula:

VH₁-CH1₁-L₁-VH₂-CH1₂ [I],
a second polypeptide chain (e.g., a first light chain polypeptide) comprises a structure represented by the formula:

VL₁-CL₁-L₂-VL₂-CL₂ [II],
a third polypeptide chain (e.g., a second heavy chain polypeptide) that comprises a structure represented by the formula:

CH1₃ [IIIa],
and a fourth polypeptide chain (e.g., a second light chain polypeptide) that comprises a structure represented by the formula:

fusion polypeptide-L₃-CL₃ [IVb]
wherein: VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, CL₃ is a third immunoglobulin light chain constant domain, VH₁ is a first immunoglobulin heavy chain variable domain, VH₂ is a second immunoglobulin heavy chain variable domain, CH1₁ is a first immunoglobulin heavy chain constant domain, CH1₂ is a second immunoglobulin heavy chain constant domain, CH1₃ is a third immunoglobulin heavy chain constant domain, and L₁, L₂, and L₃ are amino acid linkers; wherein:
a) CL₃ comprises one or more amino acid substitutions which reduce binding to a KappaSelect chromatography material compared to a CL₃ without the one or more amino acid substitutions, VL₁ and VL₂ each comprise one or more amino acid substitutions which reduce binding to a protein L chromatography material compared to a VL₁ and VL₂ without the one or more amino acid substitutions,
b) CL₃ comprises one or more amino acid substitutions which reduce binding to a KappaSelect chromatography material compared to a CL₃ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin light chain variable domain; or
c) CL₃ comprises one or more amino acid substitutions which reduce binding to a KappaSelect chromatography material compared to a CL₃ without the one or more amino acid substitutions, VL₂ comprises one or more amino acid substitutions which reduce binding to a protein L chromatography material compared to a VL₂ without the one or more amino acid substitutions, and VL₁ is a λ subtype immunoglobulin light chain variable domain or a κ2 subtype immunoglobulin light chain variable domain; and wherein VL₁ and VH₁ form a first antigen binding domain and VL₂ and VH₂ form a second antigen binding domain.

In some embodiments, at least one of L₁, L₂, or L₃ are each independently 0 amino acids in length. In some embodiments, a linker of 0 amino acids in length indicates that the linker is absent from the binding protein. In some embodiments, L₁, L₂, or L₃ are each independently at least one amino acid in length. In some embodiments, the binding of the CL₃ of the multivalent binding protein to KappaSelect chromatography material is reduced by about 90%, as compared to the binding of a CL₃ of a multivalent binding protein without the one or more amino acid substitutions. In some embodiments, the binding of the VL₁ and/or the VL₂ to a protein L chromatography material is reduced by about 90%, as compared to the binding of a VL₁ or a VL₂ without the one or more amino acid substitutions. In some embodiments, the one or more amino acid substitutions in the CL₃ of the multivalent binding protein is at a position corresponding to 109, 110 or 199, wherein numbering is according to the EU index. In some embodiments, the one or more amino acid substitutions in of CL₃ is a T109A substitution, a V110D substitution, a Q199K substitution, T109A-V110D substitutions, or T109A-V110D-Q199K substitutions, wherein numbering is according to the EU index. In some embodiments, the one or more amino acid substitutions in the CL₃ of the multivalent binding protein is at a position corresponding to 109, 198, 199, or 202, wherein numbering is according to the EU Index. In some embodiments, the one or more amino acid substitutions in the CL₃ is a H198R substitution, a Q199W substitution, or T 109A-S202R substitutions, wherein amino acid numbering is according to the EU index. In some embodiments, the one or more amino acid substitutions in the VL₁ and/or the VL₂ of the multivalent binding protein is a substitution of a framework amino acid. In some embodiments, the one or more amino acid substitutions in VL₁ and/or VL₂ is at a position corresponding to 12 or 18, wherein numbering is according to Kabat. In some embodiments, the one or more amino acid substitutions in VL₁ and/or VL₂ is a S12P substitution, a R18P substitution, a R18Q substitution, S12P-R18P substitutions, or S12P-R18Q substitutions, wherein numbering is according to Kabat.

In some embodiments, provided is a binding protein (e.g., a multivalent binding protein) comprising four polypeptide chains that form two antigen binding domains and a target binding domain; wherein the four polypeptide chains comprise:
a first polypeptide chain (e.g., a first heavy chain polypeptide) that comprises a structure represented by the formula:

VH₁-CH1₁-L₁-VH₂-CH1₂ [I],
a second polypeptide chain (e.g., a first light chain polypeptide) that comprises a structure represented by the formula:

VL₁-CL₁-L₂-VL₂-CL₂ [II],
a third polypeptide chain (e.g., a second heavy chain polypeptide) that comprises a structure represented by the formula:

fusion polypeptide-L₃-CH1₃ [III],
and a fourth polypeptide chain (e.g., a second light chain polypeptide) that comprises a structure represented by the formula:

fusion polypeptide-L₄-CL₃ [IV]
wherein: VL₁ is a first immunoglobulin light chain variable domain, VL₂ is a second immunoglobulin light chain variable domain, CL₁ is a first immunoglobulin light chain constant domain, CL₂ is a second immunoglobulin light chain constant domain, CL₃ is a third immunoglobulin light chain constant domain, VH₁ is a first immunoglobulin heavy chain variable domain, VH₂ is a second immunoglobulin heavy chain variable domain, CH1₁ is a first immunoglobulin heavy chain constant domain, CH1₂ is a second immunoglobulin heavy chain constant domain, CH1₃ is a third immunoglobulin heavy chain constant domain, and L₁, L₂, L₃ and L₄ are amino acid linkers; wherein:
a) CL₃ comprises one or more amino acid substitutions which reduce binding to a KappaSelect chromatography material compared to a CL₃ without the one or more amino acid substitutions, VL₁ and VL₂ each comprise one or more amino acid substitutions which reduce binding to a protein L chromatography material compared to a VL₁ and VL₂ without the one or more amino acid substitutions,
b) CL₃ comprises one or more amino acid substitutions which reduce binding to a KappaSelect chromatography material compared to a CL₃ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin light chain variable domain; or
c) CL₃ comprises one or more amino acid substitutions which reduce binding to a KappaSelect chromatography material compared to a CL₃ without the one or more amino acid substitutions, VL₂ comprises one or more amino acid substitutions which reduce binding to a protein L chromatography material compared to a VL₂ without the one or more amino acid substitutions, and VL₁ is a λ subtype immunoglobulin light chain variable domain or a κ2 subtype immunoglobulin light chain variable domain; and
wherein VL₁ and VH₁ form a first antigen binding domain and VL₂ and VH₂ form a second antigen binding domain. In some embodiments, each fusion polypeptide binds a target antigen. In some embodiments, the target antigens are the same. In some embodiments, the target antigens are different. In some embodiments, the fusion polypeptides dimerize and bind a single target. An example of the embodiments described above is shown in FIG. 1M.

In some embodiments, the first heavy chain polypeptide of the multivalent binding protein comprises a first CH2 immunoglobulin heavy chain constant domain and a first CH3 immunoglobulin heavy chain constant domain and the second heavy chain polypeptide of the multivalent binding protein comprises a second CH2 immunoglobulin heavy chain constant domain and a second CH3 immunoglobulin heavy chain constant domain. In some embodiments, the first CH₃ domain and/or the CH3 domain is a human IgG1 or IgG4 CH3 domain. In some embodiments, the first CH3 domain comprises amino acid substitutions at positions corresponding to (e.g., relative to) positions 354 and 366 of human IgG1, wherein numbering is according to the EU Index. In some embodiments, the amino acid substitutions are (or correspond to) S354C and T366W, wherein amino acid numbering is according to the EU index. Additionally or alternatively, in some embodiments, the second CH3 domain comprises amino acid substitutions at positions corresponding to (e.g., relative to) positions 349, 366, 368, 407, 435, and 436 of human IgG1, wherein numbering is according to the EU Index, wherein the amino acid substitutions are (or correspond to) Y349C, T366S, L368A, and Y407V, wherein amino acid numbering is according to the EU index.

In some embodiments, the second CH3 domain of the multivalent binding protein comprises (such as further comprises) one or more amino acid substitutions which reduce binding to protein A. In some embodiments, the CH3 of the first heavy chain polypeptide of the multivalent binding protein comprises (such as further comprises) one or more amino acid substitutions which reduce binding to protein A. In some embodiments, the one or more amino acid substitutions which reduce binding to a Protein A chromatography material are amino acid substitutions at positions corresponding to (e.g., relative to) positions 435 and 436 of human IgG1, wherein numbering is according to the EU Index. In some embodiments, the amino acid substitutions are (or correspond to) H435R and Y436F, wherein amino acid numbering is according to the EU index. In some embodiments, multivalent binding protein is a multispecific antibody or antigen binding fragment thereof.

Linkers

Examples of suitable linkers include a single glycine (Gly) residue; a diglycine peptide (Gly-Gly); a tripeptide (Gly-Gly-Gly); a peptide with four glycine residues (Gly-Gly-Gly-Gly (SEQ ID NO: 1)); a peptide with five glycine residues (Gly-Gly-Gly-Gly-Gly (SEQ ID NO: 2)); a peptide with six glycine residues (Gly-Gly-Gly-Gly-Gly-Gly (SEQ ID NO: 3)); a peptide with seven glycine residues (Gly-Gly-Gly-Gly-Gly-Gly-Gly (SEQ ID NO: 4)); a peptide with eight glycine residues (Gly-Gly-Gly-Gly-Gly-Gly-Gly-Gly (SEQ ID NO: 5)). Other combinations of amino acid residues may be used such as the peptide Gly-Gly-Gly-Gly-Ser (SEQ ID NO: 6), the peptide Gly-Gly-Gly-Gly-Ser-Gly-Gly-Gly-Gly-Ser (SEQ ID NO: 7), the peptide Gly-Gly-Gly-Gly-Ser-Gly-Gly-Gly-Gly-Ser-Gly-Gly-Gly-Gly-Ser (SEQ ID NO: 8), and the peptide Gly-Gly-Ser-Gly-Ser-Ser-Gly-Ser-Gly-Gly (SEQ ID NO: 6). In some embodiments, the linker is or comprises (GGGGS)n, e.g., (SEQ ID NO:6)n (wherein n is an integer between 0 and 5), e.g., GGGGSGGGGS (SEQ ID NO: 9), GGGGSGGGGSGGGGS (SEQ IS NO: 10), etc. In some embodiments, the linker is or comprises S, RT, TKGPS (SEQ ID NO: 11), GQPKAAP (SEQ ID NO: 12), or GGSGSSGSGG (SEQ ID NO: 13). Other suitable linkers include a single Ser residue or a single Val residue; the dipeptide Arg-Thr, Gln-Pro, Ser-Ser, Thr-Lys, and Ser-Leu; Thr-Lys-Gly-Pro-Ser (SEQ ID NO: 13); Thr-Val-Ala-Ala-Pro (SEQ ID NO: 14); Gln-Pro-Lys-Ala-Ala (SEQ ID NO: 15); Gln-Arg-Ile-Glu-Gly (SEQ ID NO: 16); Ala-Ser-Thr-Lys-Gly-Pro-Ser (SEQ ID NO: 17); Arg-Thr-Val-Ala-Ala-Pro-Ser (SEQ ID NO: 18); Gly-Gln-Pro-Lys-Ala-Ala-Pro (SEQ ID NO: 19); Asp-Lys-Thr-His-Thr (SEQ ID NO: 20); Gly-Gln-Pro-Lys-Ala-Ala-Pro (SEQ ID NO: 21); Thr-Lys-Gly-Pro-Ser-Arg (SEQ ID NO: 22); and His-Ile-Asp-Ser-Pro-Asn-Lys (SEQ ID NO: 23). The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins. For additional descriptions of linker sequences, see, e.g., WO2012135345.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

In some embodiments, a linker of a multivalent binding protein disclosed herein comprises a sequence derived from a naturally occurring sequence at the junction between an antibody variable domain and an antibody constant domain (e.g., as described in WO2012/135345). For example, in some embodiments, the linker comprises a sequence found at the transition between an endogenous V_{H} and C_{H1} domain, or between an endogenous V_{L} and C_{L} domain (e.g., kappa or lambda). In some embodiments, the linker comprises a sequence found at the transition between an endogenous human V_{H} and CH₁ domain, or between an endogenous human V_{L} and C_{L} domain (e.g., human kappa or lambda).

Fc Regions and Constant Domains

In some embodiments, a multivalent binding protein of the present disclosure comprises one or more Fc variants. In some examples, the term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. In some examples, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

To improve the yields of the binding proteins, the C_{H3} domains can be altered by the “knob-into-holes” technology, which is described in detail with several examples in, for example, International Publication No. WO 96/027011, Ridgway et al., 1996, Protein Eng. 9: 617-21; and Merchant et al., 1998, Nat. Biotechnol. 16: 677-81. Specifically, the interaction surfaces of the two CH₃ domains are altered to increase the heterodimerization of both heavy chains containing these two CH3 domains. Each of the two CH3 domains (of the two heavy chains) can be the “knob,” while the other is the “hole.” The introduction of a disulfide bridge further stabilizes the heterodimers (Merchant et al., 1998; Atwell et al., 1997, J. Mol. Biol. 270: 26-35) and increases the yield. In particular embodiments, the knob is on the second pair of polypeptides with a single variable domain. In other embodiments, the knob is on the first pair of polypeptides having the cross-over orientation. In yet other embodiments, the CH3 domains do not include a knob in hole. In some embodiments, the CH3 domain of the first heavy chain polypeptide comprises amino acid substitutions at positions corresponding to (e.g., relative to) positions 354 and 366 of human IgG1, wherein numbering is according to the EU Index. See, e.g., Spiess et al. (2013) JBC, 288(37): P26583-26593. In some embodiments, the amino acid substitutions are (or correspond to) S354C and T366W (i.e., knob substitutions), wherein amino acid numbering is according to the EU index. Additionally or alternatively, in some embodiments, the CH3 domain of the second heavy chain polypeptide comprises amino acid substitutions at positions corresponding to (e.g., relative to) positions 349, 366, 368, 407, 435, and 436 of human IgG1, wherein numbering is according to the EU Index. In some embodiments, the amino acid substitutions are (or correspond to) Y349C, T366S, L368A, and Y407V (i.e., hole substitutions) wherein amino acid numbering is according to the EU index.

In some embodiments, a binding protein of the present disclosure comprises one or more mutations to improve serum half-life (see, e.g., Hinton, P. R. et al. (2006) J. Immunol. 176(1):346-56). In some embodiments, the mutation comprises substitutions at positions corresponding to (e.g., such as relative to) positions 428 and 434 of human IgG1, wherein numbering is according to the EU Index, wherein the amino acid substitutions are (or correspond to) M428L and N434S wherein amino acid numbering is according to the EU index.

In some embodiments, a binding protein of the present disclosure comprises one or more mutations to improve purification, e.g., by modulating the affinity for a chromatography material. For example, it is known that heterodimeric binding proteins can be selectively purified away from their homodimeric forms if one of the two Fc regions of the heterodimeric form contains mutation(s) that reduce or eliminate binding to Protein A, because the heterodimeric form will have an intermediate affinity for Protein A-based purification than either homodimeric form and can be selectively eluted from Protein A, e.g., by use of a different pH (see, e.g., Smith, E. J. et al. (2015) Sci. Rep. 5:17943). In some embodiments, the mutation comprises substitutions at positions corresponding to (e.g., such as relative to) positions 435 and 436 of human IgG1, wherein numbering is according to the EU Index, wherein the amino acid substitutions are (or correspond to) H435R and Y436F wherein amino acid numbering is according to the EU index.

In some embodiments, a multivalent binding protein of the present disclosure comprises one or more mutations to reduce effector function, e.g., Fc receptor-mediated antibody-dependent cellular phagocytosis (ADCP), complement-dependent cytotoxicity (CDC), and/or antibody-dependent cellular cytotoxicity (ADCC). In some embodiments, the mutations comprise amino acid substitutions at positions corresponding to (e.g., relative to) positions 234, 235, and/or 239 of human IgG1, wherein numbering is according to the EU Index. In some embodiments, the amino acid substitutions are (or correspond to) L234A, L235A, and/or P329A, wherein amino acid numbering is according to the EU index.

In some embodiments, the types of mutations described supra can be combined in any order or combination. For example, a binding protein of the present disclosure can comprise two or more of the “knob” and “hole” mutations, the one or more mutations to improve serum half-life, the one or more mutations to improve IgG4 stability, the one or more mutations to improve purification, and/or the one or more mutations to reduce effector function described supra.

### Methods of Purifying Multivalent Binding Proteins

Exemplary Chromatography Materials

Protein A

Staphylococcal protein A (SPA or “Protein A”) is one of the first discovered immunoglobulin binding molecules and has been extensively studied during the past decades. Due to its affinity to immunoglobulins, Protein A has found widespread use as a tool in the detection and purification of antibodies, antibody constructs, and Fc-containing fusion proteins. composed of five immunoglobulin-binding domains, each of which are able to bind proteins from many mammalian species, most notably Immunoglobulin G (IgG) through the heavy chain within the Fc region. While the native form of Protein A was used as the ligand for first generation Protein A resins, recombinant forms (rProtein A) produced in E. coli are the most prevalent today. Commercially available Protein A chromatography materials include, but are not limited to, those detailed herein below.

KappaSelect

KappaSelect is an affinity chromatography material that specifically binds to the constant region of the kappa light chain (LC). The KappaSelect ligand comprises a camelid single-variable heavy-chain (HC) Ig (VHH) domain that binds with high affinity to all human Cκ-LC independent of VL sequences. The KappaSelect ligand interacts primarily with residues in Cκ and to some extent with residues in Vκ-Cκ hinge region. Commercially available KappaSelect chromatography materials include, but are not limited to, those detailed herein below.

Protein L

Protein L is a cell wall protein of the bacterium Peptostreptococcus magnus (Björck et al. (1988) “Novel Bacterial-Cell Wall Protein with Affinity for Ig L-Chains.” J. Immunol. 140: 1194-1197) that binds to the variable region of the kappa light chain without interfering with the antigen binding site (Nilson et al. (1992) “Protein L from Peptostreptococcus magnus binds to the kappa light chain variable domain.” J Biol Chem. 267: 2234-2239) of an antibody or antibody construct (e.g., a multivalent binding protein described herein). Protein L interacts with FW1 in V-region of a kappa light chain, and its binding is restricted to VL of κ1, κ3 and κ4 subtypes. Commercially available Protein L chromatography materials include, but are not limited to, those detailed herein below.

Exemplary Two Step Purification Process

Provided herein is a method of purifying a multivalent binding protein described herein, which method comprises (a) subjecting a composition comprising the multivalent binding protein to Protein L chromatography in bind and elute mode to generate a protein L eluate, and (b) subjecting the protein L eluate to KappaSelect chromatography in bind and elute chromatography to generate a KappaSelect eluate, wherein the KappaSelect eluate comprises the multivalent binding protein and is essentially free of mispaired polypeptides. For example, in some embodiments, the multivalent binding protein is contacted with Protein L under conditions suitable for isolating the binding protein away from undesirable protein species comprising either 0 or 2 VL domains that comprise one or more amino acid substitutions that reduce the binding of the VL to a protein L chromatography material (e.g., as compared to a VL domain without the one or more amino acid substitutions). The Protein L eluate thus generated is then contacted with a KappaSelect chromatography material under conditions suitable for isolating the binding protein away from undesirable protein species comprising either 0 or 2 CL domains that comprise one or more amino acid substitutions that reduce the binding of the CL to a KappaSelect chromatography material (e.g., as compared to a CL domain without the one or more amino acid substitutions). Also provided herein is a method of purifying a multivalent binding protein disclosed herein, the method comprising (a) subjecting a composition comprising the multivalent binding protein and mispaired antibodies to KappaSelect chromatography in bind and elute mode to generate as KappaSelect eluate and (b) subjecting the KappaSelect eluate to Protein L chromatography in bind and elute mode to generate a protein L eluate, wherein the protein L eluate comprises the multivalent binding protein and is essentially free of mispaired polypeptides.

Conditions suitable for using Protein L and KappaSelect chromatography materials in bind and elute mode known in the art. In some embodiments, the Protein L and/or the KappaSelect is attached to a substrate or resin, e.g., as part of a chromatography material. In some embodiments, the Protein L chromatography is a Pierce™ Protein L chromatography cartridge, a Capto™ L chromatography, HiTrap® Protein L chromatography, a TOYOPEARL® AF-rProtein L-650F chromatography, or a KanCap™ L chromatography. Additionally or alternatively, in some embodiments of any of the purification processes disclosed herein, the KappaSelect chromatography is a HiTrap™ KappaSelect or a CaptureSelect™ Kappa XL chromatography.

The KappaSelect eluate comprises the multivalent binding protein and is essentially free of mispaired polypeptides. In some embodiments, the multivalent binding protein in the KappaSelect eluate is at least about any one of 80%, 81%, 82% 83%, 84%, 85%, 86%, 87%, 88%, 89%, 90%, 91%, 92%, 93%, 94%, 95%, 96%, 97%, 98%, or 99% pure, including any range in between these values. In some embodiments, the multivalent binding protein in the KappaSelect eluate is more than about 99% pure. In some embodiments, less than about any of 15%, 14%, 13%, 12%, 11%, 10%, 9%, 8%, 7%, 6%, 5% 4%, 3%, 2%, or 1% of the multivalent binding polypeptides in the KappaSelect eluate are mispaired polypeptides. In some embodiments, less than about 1% of the multivalent binding protein in the KappaSelect eluate are mispaired polypeptides.

Exemplary Three Step Purification Process

Provided herein is a method of purifying a multivalent binding protein described herein, which method comprises (a) subjecting a composition comprising the multivalent binding protein to Protein A chromatography in bind and elute mode to generate a Protein A eluate, (b) subjecting the Protein A eluate to Protein L chromatography in bind and elute mode to generate a protein L eluate, and (c) subjecting the protein L eluate to KappaSelect chromatography in bind and elute mode to generate a KappaSelect eluate, wherein the KappaSelect eluate comprises the multivalent binding protein and is essentially free of mispaired polypeptides. For example, in some embodiments, the multivalent binding protein is contacted with Protein A under conditions for isolating the binding protein away from undesirable protein species that comprise 2 CH3 domains that each comprise Y349C, T366S, L368A, and Y407V substitutions (relative to human IgG1), wherein amino acid numbering is according to the EU index, and wherein the. In some embodiments, the multivalent binding protein is contacted with Protein A under conditions for isolating the binding protein away from undesirable protein species that comprise 2 CH3 domains that each comprise H435R and Y436F substitutions (relative to human IgG1), wherein amino acid numbering is according to the EU index. In some embodiments, the multivalent binding protein is contacted with Protein A under conditions for isolating the binding protein away from undesirable protein species that comprise 2 CH3 domains that each comprise Y349C, T366S, L368A, and Y407V substitutions and H435R and Y436F substitutions (relative to human IgG1), wherein amino acid numbering is according to the EU index. In some embodiments, the second CH3 domain of the multivalent binding protein comprises (such as further comprises) one or more amino acid substitutions which reduce binding to protein A. In some embodiments, the CH3 of the first heavy chain polypeptide of the multivalent binding protein comprises (such as further comprises) one or more amino acid substitutions which reduce binding to protein A. In some embodiments, the one or more amino acid substitutions which reduce binding to a Protein A chromatography material are (or correspond to) amino acid substitutions at positions corresponding to positions 435 and 436 of human IgG1, wherein numbering is according to the EU index. In some embodiments, the amino acid substitutions are (or correspond to) H435R and Y436F, wherein amino acid numbering is according to the EU index. In some embodiments, multivalent binding protein is a multispecific antibody or antigen binding fragment thereof.

The Protein A eluate is then contacted with Protein L under conditions suitable for isolating the binding protein away from undesirable protein species comprising either 0 or 2 VL domains that comprise one or more amino acid substitutions that reduce the binding of the VL to a protein L chromatography material (e.g., as compared to a VL domain without the one or more amino acid substitutions). The Protein L eluate is then contacted with a KappaSelect chromatography material under conditions suitable for isolating the binding protein away from undesirable protein species comprising either 0 or 2 CL domains that comprise one or more amino acid substitutions that reduce the binding of the CL to a KappaSelect chromatography material (e.g., as compared to a CL domain without the one or more amino acid substitutions). Also provided is a method of purifying a multivalent binding protein described herein, which method comprises (a) subjecting a composition comprising the multivalent binding protein to Protein A chromatography in bind and elute mode to generate a Protein A eluate, (b) subjecting the Protein A eluate to KappaSelect chromatography in bind and elute mode to generate a KappaSelect eluate, and (c) subjecting the protein KappaSelect eluate to Protein L chromatography in bind and elute mode to generate a protein L eluate, wherein the L eluate comprises the multivalent binding protein and is essentially free of mispaired polypeptides.

Conditions suitable for the use of Protein A, Protein L, and KappaSelect chromatography materials in bind and elute mode are known in the art. In some embodiments, the Protein A, Protein L, and or KappaSelect ligand is attached to a substrate or resin, e.g., as part of a chromatography material. In some embodiments of any of the purification processes disclosed herein, the Protein A chromatography is a MabSelect™, MabSelect SuRe™, MabSelect SuRe™ LX, MabSelect PrismA, ProSep®-vA, ProSep® Ultra Plus, Protein A Sepharose® Fast Flow, or Toyopearl® AF-rProtein A chromatography. In some embodiments, the Protein L chromatography is a Pierce™ Protein L chromatography cartridge, a Capto™ L chromatography, HiTrap® Protein L chromatography, a TOYOPEARL® AF-rProtein L-650F chromatography, or a KanCap™ L chromatography. Additionally or alternatively, in some embodiments of any of the purification processes disclosed herein, the KappaSelect chromatography is a HiTrap™ KappaSelect or a CaptureSelect™ Kappa XL chromatography.

The KappaSelect eluate comprises the multivalent binding protein and is essentially free of mispaired polypeptides. In some embodiments, the multivalent binding protein in the KappaSelect eluate is at least about any one of 80%, 81%, 82% 83%, 84%, 85%, 86%, 87%, 88%, 89%, 90%, 91%, 92%, 93%, 94%, 95%, 96%, 97%, 98%, or 99% pure, including any range in between these values. In some embodiments, the multivalent binding protein in the KappaSelect eluate is more than about 99% pure. In some embodiments, less than about any of 15%, 14%, 13%, 12%, 11%, 10%, 9%, 8%, 7%, 6%, 5% 4%, 3%, 2%, or 1% of the multivalent binding polypeptides in the KappaSelect eluate are mispaired polypeptides. In some embodiments, less than about 1% of the multivalent binding protein in the KappaSelect eluate are mispaired polypeptides.

In some embodiments of any of the purification processes disclosed herein, the composition comprising the multivalent binding protein is derived from a host cell engineered to produce the multispecific binding protein. In some embodiments, the composition comprising the multivalent binding protein is a host cell culture supernatant. In some embodiments, the composition comprising the multivalent binding protein further comprises mispaired polypeptides. In some embodiments, the composition comprising the multivalent binding protein is filtered prior to chromatography (e.g., a first chromatography step). In some embodiments, a purification process disclosed herein further comprises a polishing step after the KappaSelect chromatography or protein L chromatography. In some embodiments, the polishing step is a size exclusion chromatography.

In some embodiments of any of the purification processes disclosed herein, the composition comprising the multivalent binding protein is combined with a pharmaceutically acceptable carrier. Exemplary pharmaceutically acceptable carriers and excipients are described in father detail elsewhere herein.

Methods of Producing a Multivalent Binding Protein with Reduced Binding to a KappaSelect and/or a Protein L Chromatography Material

Nucleic Acids and Vectors

Nucleic acid molecules encoding multivalent binding proteins described herein are also contemplated. In some embodiments, provided is a nucleic acid (or a set of nucleic acids) encoding an multivalent binding protein described herein. Standard recombinant DNA methodologies are used to construct the polynucleotides that encode the polypeptides which form the binding proteins, incorporate these polynucleotides into recombinant expression vectors, and introduce such vectors into host cells. See, e.g., Sambrook et al., 2001, MOLECULAR CLONING: A LABORATORY MANUAL (Cold Spring Harbor Laboratory Press, 3rd ed.). Enzymatic reactions and purification techniques may be performed according to manufacturer's specifications, as commonly accomplished in the art, or as described herein. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

Other aspects of the present disclosure relate to isolated nucleic acid molecules comprising a nucleotide sequence encoding any of the binding proteins (e.g., multivalent binding proteins) described herein. In some embodiments, the isolated nucleic acid is operably linked to a heterologous promoter to direct transcription of the binding protein-coding nucleic acid sequence. A promoter may refer to nucleic acid control sequences which direct transcription of a nucleic acid. A first nucleic acid sequence is operably linked to a second nucleic acid sequence when the first nucleic acid sequence is placed in a functional relationship with the second nucleic acid sequence. For instance, a promoter is operably linked to a coding sequence of a binding protein if the promoter affects the transcription or expression of the coding sequence. Examples of promoters may include, but are not limited to, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), from heterologous eukaryotic promoters (such as the actin promoter, an immunoglobulin promoter, from heat-shock promoters, and the like), the CAG-promoter (Niwa et al., Gene 108(2):193-9, 1991), the phosphoglycerate kinase (PGK)-promoter, a tetracycline-inducible promoter (Masui et al., Nucleic Acids Res. 33:e43, 2005), the lac system, the trp system, the tac system, the trc system, major operator and promoter regions of phage lambda, the promoter for 3-phosphoglycerate kinase, the promoters of yeast acid phosphatase, and the promoter of the yeast alpha-mating factors. Polynucleotides encoding binding proteins of the present disclosure may be under the control of a constitutive promoter, an inducible promoter, or any other suitable promoter described herein or other suitable promoter that will be readily recognized by one skilled in the art.

In some embodiments, the isolated nucleic acid is incorporated into a vector. In some embodiments, the vector is an expression vector. Expression vectors may include one or more regulatory sequences operatively linked to the polynucleotide to be expressed. The term “regulatory sequence” includes promoters, enhancers and other expression control elements (e.g., polyadenylation signals). Examples of suitable enhancers may include, but are not limited to, enhancer sequences from mammalian genes (such as globin, elastase, albumin, α-fetoprotein, insulin and the like), and enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like). Examples of suitable vectors may include, for example, plasmids, cosmids, episomes, transposons, and viral vectors (e.g., adenoviral, vaccinia viral, Sindbis-viral, measles, herpes viral, lentiviral, retroviral, adeno-associated viral vectors, etc.). Expression vectors can be used to transfect host cells, such as, for example, bacterial cells, yeast cells, insect cells, and mammalian cells. Biologically functional viral and plasmid DNA vectors capable of expression and replication in a host are known in the art, and can be used to transfect any cell of interest.

Other aspects of the present disclosure relate to a vector system comprising one or more vectors encoding a first, second, third, and fourth polypeptide chain of any of the binding proteins described herein. In some embodiments, the vector system comprises a first vector encoding the first polypeptide chain of the binding protein, a second vector encoding the second polypeptide chain of the binding protein, a third vector encoding the third polypeptide chain of the binding protein, and a fourth vector encoding the fourth polypeptide chain of the binding protein. In some embodiments, the vector system comprises a first vector encoding the first and second polypeptide chains of the binding protein, and a second vector encoding the third and fourth polypeptide chains of the binding protein. In some embodiments, the vector system comprises a first vector encoding the first and third polypeptide chains of the binding protein, and a second vector encoding the second and fourth polypeptide chains of the binding protein. In some embodiments, the vector system comprises a first vector encoding the first and fourth polypeptide chains of the binding protein, and a second vector encoding the second and third polypeptide chains of the binding protein. In some embodiments, the vector system comprises a first vector encoding the first, second, third, and fourth polypeptide chains of the binding protein. The one or more vectors of the vector system may be any of the vectors described herein. In some embodiments, the one or more vectors are expression vectors.

Isolated Host Cells

Other aspects of the present disclosure relate to an isolated host cell comprising one or more isolated polynucleotides, vectors, and/or vector systems described herein. In some embodiments, the host cell is a bacterial cell (e.g., an E. coli cell). In some embodiments, the host cell is a yeast cell (e.g., an S cerevisiae cell). In some embodiments, the host cell is an insect cell. Examples of insect host cells may include, for example, Drosophila cells (e.g., S2 cells), Trichoplusia ni cells (e.g., High Five™ cells), and Spodoptera frugiperda cells (e.g., Sf21 or Sf9 cells). In some embodiments, the host cell is a mammalian cell. Examples of mammalian host cells may include, for example, human embryonic kidney cells (e.g., 293 or 293 cells subcloned for growth in suspension culture), Expi293TM cells, CHO cells, baby hamster kidney cells (e.g., BHK, ATCC CCL 10), mouse sertoli cells (e.g., TM4 cells), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor cells (e.g., MMT 060562, ATCC CCL51), TRI cells, MRC 5 cells, FS4 cells, a human hepatoma line (e.g., Hep G2), and myeloma cells (e.g., NS0 and Sp2/0 cells).

Other aspects of the present disclosure relate to a method of producing any of the binding proteins described herein. In some embodiments, the method includes a) culturing a host cell (e.g., any of the host cells described herein) comprising an isolated nucleic acid, vector, and/or vector system (e.g., any of the isolated nucleic acids, vectors, and/or vector systems described herein) under conditions such that the host cell expresses the binding protein; and b) isolating the binding protein from the host cell. Methods of culturing host cells under conditions to express a protein are well known to one of ordinary skill in the art. Methods of isolating a multivalent binding protein described herein from cultured host cells are well known to one of ordinary skill in the art. Methods of purifying a multivalent binding protein described herein are detailed elsewhere herein.

### Exemplary Uses for Multivalent Binding Proteins

The multivalent binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in certain embodiments, binding proteins can be labeled with a detectable moiety. The detectable moiety can be any one that is capable of producing, either directly or indirectly, a detectable signal. For example, the detectable moiety can be a radioisotope, such as ³H, ⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The binding proteins are also useful for in vivo imaging. A binding protein labeled with a detectable moiety can be administered to an animal, preferably into the bloodstream, and the presence and location of the labeled antibody in the host assayed. The binding protein can be labeled with any moiety that is detectable in an animal, whether by nuclear magnetic resonance, radiology, or other detection means known in the art.

The binding proteins can also be used for cell activation, tumor targeting, neutralization of cytokine activities, neutralization of viral infection, combination of multiple signaling events, to treat cancer, arthritis, and/or inflammatory disorders. For example, in some embodiments, a binding protein specifically binds one, two, or three antigen targets selected from A2AR, APRIL, ATPDase, BAFF, BAFFR, BCMA, BlyS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-1a), CCL4 (also known as MIP-1b), CCL5 (also known as RANTES), CCL7 (also known as MCP-3), CCL8 (also known as mcp-2), CCL11 (also known as eotaxin), CCL15 (also known as MIP-Id), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CD19, CD20, CD23 (also known as FCER2, a receptor for IgE), CD24, CD27, CD28, CD38, CD39, CD40, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDL1), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL₁ (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, ILlA, ILIB, ILIF10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb (also known as a receptor for IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, NKG2D, NTPDase-1, OX40, OX40L, PD-1H, platelet receptor, PROMI, S152, SISPI, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TNFRSF7, Tp55, TREMI, TSLP (also known as a co-receptor for IL7Ra), TSLPR, TWEAK, VEGF, VISTA, Vstm3, WUCAM, and XCR1 (also known as GPR5/CCXCR1). In some embodiments, one or more of the above antigen targets are human antigen targets.

In some embodiments, a binding protein of the present disclosure is administered to a patient in need thereof for the treatment or prevention of cancer. For example, in some embodiments, the binding protein comprises one antigen binding site that specifically binds a T-cell surface protein and another antigen binding site that specifically binds a tumor target protein (e.g., two antigen binding sites that specifically bind T-cell surface proteins and one antigen binding site that specifically binds a tumor target protein, or two antigen binding sites that specifically bind tumor target proteins and one antigen binding site that specifically binds a T-cell surface protein). In certain embodiments, the binding protein comprises an antigen binding site that specifically binds CD3, an antigen binding site that specifically binds CD28, and an antigen binding site that specifically binds a tumor target protein selected from CD19, CD20, CD38, Her2, and LAMP1. In some embodiments, the binding protein is co-administered with a chemotherapeutic agent. In some embodiments, the patient is a human.

In some embodiments, a binding protein of the present disclosure is administered to a patient in need thereof for the treatment or prevention of an inflammatory disease or disorder. In some embodiments, the binding protein comprises three antigen binding sites that each specifically bind a cytokine target protein selected from IL-4, IL-13 and TNFa. In some embodiments, the binding protein is co-administered with an anti-inflammatory agent. In some embodiments, the patient is a human.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Therapeutic Compositions Comprising Multivalent Binding Proteins and Administration Thereof

Therapeutic or pharmaceutical compositions comprising binding proteins are within the scope of the disclosure. Such therapeutic or pharmaceutical compositions can comprise a therapeutically effective amount of a binding protein, or binding protein-drug conjugate, in admixture with a pharmaceutically or physiologically acceptable formulation agent selected for suitability with the mode of administration.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery or subcutaneous. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(-)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single-dose administration unit. The kits can each contain both a first container having a dried protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

In some embodiments, the present disclosure relates to a method of preventing and/or treating a proliferative disease or disorder (e.g., cancer). In some embodiments, the method comprises administering to a patient a therapeutically effective amount of at least one of the binding proteins described herein. In some embodiments, the patient is a human. In some embodiments, the at least one binding protein is administered in combination with one or more anti-cancer therapies (e.g., any anti-cancer therapy known in the art). In some embodiments, the at least one binding protein is administered before the one or more anti-cancer therapies. In some embodiments, the at least one binding protein is administered concurrently with the one or more anti-cancer therapies. In some embodiments, the at least one binding protein is administered after the one or more anti-retroviral therapies.

In some embodiments, the present disclosure relates to a method of preventing and/or treating an inflammatory disease or disorder (e.g., cancer). In some embodiments, the method comprises administering to a patient a therapeutically effective amount of at least one of the binding proteins described herein. In some embodiments, the patient is a human. In some embodiments, the at least one binding protein is administered in combination with one or more anti-inflammatory therapies (e.g., any anti-inflammatory therapy known in the art). In some embodiments, the at least one binding protein is administered before the one or more anti-inflammatory therapies. In some embodiments, the at least one binding protein is administered concurrently with the one or more anti-inflammatory therapies. In some embodiments, the at least one binding protein is administered after the one or more anti-inflammatory therapies.

Without limiting the present disclosure, a number of embodiments of the present disclosure are described below for purpose of illustration.

### Exemplary Embodiments

The invention provides the following enumerated exemplary embodiments.
1. A multivalent binding protein comprising four polypeptide chains that form two antigen binding domains; wherein the four polypeptide chains comprise:
a first heavy chain polypeptide comprising a structure represented by the formula:

VH1-CH1 [I],
first light chain polypeptide chain comprising a structure represented by the formula:

VL1-CL1[II],a
second heavy chain polypeptide comprising a structure represented by the formula:

VH2-CH1 [III],
and a second light chain polypeptide chain comprising a structure represented by the formula:

VL2-CL2 [IV];
wherein:
VL1 is a first immunoglobulin light chain variable domain;
VL2 is a second immunoglobulin light chain variable domain, wherein VL2 is a κ1, κ3, or κ4 subtype light chain variable domain,
CL₁ is a first immunoglobulin light chain constant domain, wherein CL₁ is a CK subtype light chain constant domain;
CL₂ is a second immunoglobulin light chain constant domain;
VH1 is a first immunoglobulin heavy chain variable domain;
VH₂ is a second immunoglobulin heavy chain variable domain;
CH₁ is an immunoglobulin heavy chain constant domain;
wherein CL₁ comprises one or more amino acid substitutions which reduce binding to a KappaSelect chromatography material compared to a CL₁ without the one or more amino acid substitutions,
wherein VL₂ comprises one or more amino acid substitutions which reduce binding to a protein L chromatography material compared to a VL₂ without the one or more amino acid substitutions, and
wherein VH₁ and VL₁ associate to form a first antigen binding domain and VH₂ and VL₂ associate to form a second antigen binding domain.
2. A multivalent binding protein comprising four polypeptide chains that form two antigen binding domains; wherein the four polypeptide chains comprise:
a first heavy chain polypeptide comprising a structure represented by the formula:

VH1-CH1-CH2-CH3 [I],
a first light chain polypeptide chain comprising a structure represented by the formula:

VL1-CL1 [II],
a second heavy chain polypeptide comprising a structure represented by the formula:

VH2-CH1-CH2-CH3 [III],
and a second light chain polypeptide chain comprising a structure represented by the formula:

VL2-CL2 [IV];
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
VH1 is a first immunoglobulin heavy chain variable domain;
VH2 is a second immunoglobulin heavy chain variable domain;
CH1 is an immunoglobulin CH1 heavy chain constant domain,
CH2 is an immunoglobulin CH2 heavy chain constant domain and
CH3 is an immunoglobulin CH3 heavy chain constant domain;
wherein CL1 comprises one or more amino acid substitutions which reduce binding to a KappaSelect chromatography material compared to a CL1 without the one or more amino acid substitutions,
wherein VL2 comprises one or more amino acid substitutions which reduce binding to a protein L chromatography material compared to a VL₂ without the one or more amino acid substitutions, and
   wherein VH1 and VL1 associate to form a first antigen binding domain and VH2 and VL2 associate to form a second antigen binding domain.
3. The multivalent binding protein of embodiment 1 or embodiment 2, wherein binding of the CL1 that comprises the one or more substitutions to KappaSelect is reduced by about 90% compared to a CL1 without the one or more amino acid substitutions.
4. The multivalent binding protein of any one of embodiments 1-3 wherein binding of the VL2 that comprises the one or more substitutions to the protein L chromatography material is reduced by about 90% compared to a VL2 without the one or more amino acid substitutions.
5. The multivalent binding protein of any one of embodiments 1-4, wherein the one or more amino acid substitutions in the CL1 that comprises the one or more substitutions is at a position corresponding to 109, 110 or 199, wherein numbering is according to the EU index.
6. The multivalent binding protein of embodiment 5, wherein the one or more amino acid substitutions in the CL1 that comprises the one or more substitutions is a T109A substitution, a V110D substitution, a Q199K substitution, T109A-V110D substitutions, or T 109A-V110D-Q199K substitutions, wherein amino acid numbering is according to the EU index.
7. The multivalent binding protein of any one of embodiments 1-4, wherein the one or more amino acid substitutions in the CL1 that comprises the one or more substitutions is at a position corresponding to 109, 198, 199, or 202, wherein numbering is according to the EU index.
8. The multivalent binding protein of embodiment 7, wherein the one or more amino acid substitutions in the CL1 that comprises the one or more substitutions is a H198R substitution, a Q199W substitution, or T109A-S202R substitutions, wherein amino acid numbering is according to the EU index.
9. The multivalent binding protein of any one of embodiments 1-8, wherein the one or more amino acid substitutions in the VL2 that comprises the one or more substitutions is a substitution of a framework amino acid.
10. The multivalent binding protein of any one of embodiments 1-9, wherein the one or more amino acid substitutions in the VL2 that comprises the one or more substitutions is at a position corresponding to 12 or 18, wherein numbering is according to Kabat.
11. The multivalent binding protein of embodiment 10, wherein the one or more amino acid substitutions in the VL2 that comprises the one or more substitutions is a S12P substitution, a R18P substitution, a R18Q substitution, S12P-R18P substitutions, or S12P-R18Q substitutions, wherein numbering is according to Kabat.
12. The multivalent binding protein of any one of embodiments 2-11, wherein the CH3 domain of the first heavy chain polypeptide and/or the CH3 domain of the second heavy chain polypeptide is a human IgG1 or IgG4 CH3 domain.
13. The multivalent binding protein of any one of embodiments 2-12, wherein the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V.
14. The multivalent binding protein of any one of embodiments 2-13, wherein the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W.
15. The multivalent binding protein of embodiment 13, wherein the CH3 of the second heavy chain polypeptide comprises one or more amino acid substitutions that reduce binding to protein A.
16. The multivalent binding protein of embodiment 14, wherein the CH3 of the first heavy chain polypeptide comprises one or more amino acid substitutions that reduce binding to protein A.
17. The multivalent binding protein of claim 15 or 16, wherein one or more amino acid substitutions that reduce binding to Protein A are amino acid substitutions at positions corresponding to positions 435 and 436 of human IgG1, wherein numbering is according to the EU Index.
18. The multivalent binding protein of claim 17, wherein the amino acid substitutions are H435R and Y436F, wherein amino acid numbering is according to the EU index.
19. The multivalent binding protein of any one of claims 2-18, wherein the CH1, CH2 and CH₃ domains of the first heavy chain polypeptide are different from the CH1, CH2 and CH₃ domains of the second heavy chain polypeptide.
20. The multivalent binding protein of any one of claims 2-19, wherein the first heavy chain polypeptide is derived from a different species than the second heavy chain polypeptide.
21. The multivalent binding protein of any one of claims 2-11, 19 or 20, wherein the first heavy chain polypeptide and the first light chain polypeptide are derived from a mouse heavy chain immunoglobulin and a mouse light chain immunoglobulin, and the second heavy chain polypeptide and the second light chain polypeptides are derived from a rat heavy chain immunoglobulin and a rat light chain immunoglobulin.
22. The multivalent binding protein of any one of claims 2-11, wherein the first heavy chain polypeptide and the second heavy chain polypeptide each comprise an IgG4 CH3 domain.
23. The multivalent binding protein of claim 22, wherein the first heavy chain polypeptide comprises a κ409R amino acid substitution and the second heavy chain polypeptide comprises a F405L amino acid substitution, wherein numbering is according to the EU index.
24. The multivalent binding protein of any one of claims 1-23, wherein the multivalent binding protein is a bispecific antigen binding protein.
25. The multivalent binding protein of any one of claims 1-24, wherein the first antigen binding domain and the second antigen binding domain bind different antigens.
26. The multivalent binding protein of any one of claims 2-18, wherein the first heavy chain polypeptide chain comprises a structure represented by the formula:

VH1-CH1-CH2-CH3-VH3-L-VL3 [Ia],
wherein the second heavy chain polypeptide comprising a structure represented by the formula:

VH2-CH1-CH2-CH3 [IIIa],
wherein:
   VL3 is a third immunoglobulin light chain variable domain;
   VH3 is a third immunoglobulin heavy chain variable domain;
   L is an amino acid linker;
   wherein VH3 and VL3 associate to form a third antigen binding domain.
27. The multivalent binding protein of any one of claims 2-18,
wherein the first heavy chain polypeptide chain comprises a structure represented by the formula:

VH1-CH1-CH2-CH3-VH3 [Ib],
wherein the second heavy chain polypeptide comprising a structure represented by the formula:

VH2-CH1-CH2-CH3 [IIIb],
wherein:
VH3 is a third immunoglobulin heavy chain variable domain
28. The multivalent binding protein of claim 26, wherein VL3 comprises one or more amino acid substitutions that reduce binding to the protein L chromatography material compared to a VL3 without the one or more amino acid substitutions.
29. The multivalent binding protein of claim 26, wherein VL3 is a λ subtype immunoglobulin light chain variable domain or a κ2 immunoglobulin light chain variable domain.
30. The multivalent binding protein of any one of claims 26-29, wherein the multivalent binding protein is bispecific or trispecific.
31. The multivalent binding protein of any one of claims 26-30, wherein the first antigen binding domain, the second antigen binding domain bind, and the third antigen binding domains bind two or three different antigens.
32. The multivalent binding protein of any one of claims 26-30, wherein the first antigen binding domain binds a first antigen, the second antigen binding domain binds a second antigen, and the third antigen binding domain binds a third antigen.
33. The multivalent binding protein of any one of claims 26-30, wherein the first antigen binding domain and the second antigen binding domain bind a first antigen and the third antigen binding domain binds a second antigen
34. A multivalent binding protein comprising four polypeptide chains that form three antigen binding domains; wherein the four polypeptide chains comprise:
a first heavy chain polypeptide comprising a structure represented by the formula:

VH1-L3-VH2-L4-CH1 [I],
a first light chain polypeptide chain comprising a structure represented by the formula:

VL2-L1-VL1-L2-CL1 [II],
a second heavy chain polypeptide comprising a structure represented by the formula:

VH3-CH1 [III],
and a second light chain polypeptide chain comprising a structure represented by the formula:

VL3-CL2 [IV]
wherein:
   VL1 is a first immunoglobulin light chain variable domain;
   VL2 is a second immunoglobulin light chain variable domain;
   VL3 is a third immunoglobulin light chain variable domain;
   VH1 is a first immunoglobulin heavy chain variable domain;
   VH2 is a second immunoglobulin heavy chain variable domain;
   VH3 is a third immunoglobulin heavy chain variable domain;
   CL1 is a first immunoglobulin light chain constant domain;
   CL2 is a second immunoglobulin light chain constant domain;
   CH1 is an immunoglobulin CH1 heavy chain constant domain; and
   L1, L2, L3 and L4 are amino acid linkers;
   wherein the polypeptide of formula I and the polypeptide of formula II form a cross-over light chain-heavy chain pair;
   wherein VH1 and VL1 associate to form a first antigen binding domain, VH2 and VL2 associate to form a second antigen binding domain, and VH3 and VL3 associate to form a third antigen binding domain; and wherein:
   a) CL1 comprises one or more amino acid substitutions which reduce binding to a KappaSelect chromatography material compared to a CL1 without the one or more amino acid substitutions and VL3 comprises one or more amino acid substitutions which reduce binding to a protein L chromatography material compared to a VL3 without the one or more amino acid substitutions;
   b) CL2 comprises one or more amino acid substitutions which reduce binding to a KappaSelect chromatography material compared to a CL2 without the one or more amino acid substitutions and VL1 and VL2 each comprise one or more amino acid substitutions which reduce binding to a protein L chromatography material compared to a VL₁ and VL2 without the one or more amino acid substitutions;
   c) CL2 comprises one or more amino acid substitutions which reduce binding to a KappaSelect chromatography material compared to a CL2 without the one or more amino acid substitutions, VL1 comprises one or more amino acid substitutions which reduce binding to a protein L chromatography material compared to a VL1 without the one or more amino acid substitutions, and wherein VL2 is a λ subtype immunoglobulin light chain variable domain or a κ2 subtype immunoglobulin light chain variable domain; or
   d) CL2 comprises one or more amino acid substitutions which reduce binding to a KappaSelect chromatography material compared to a CL2 without the one or more amino acid substitutions, VL2 comprises one or more amino acid substitutions which reduce binding to a protein L chromatography material compared to a VL2 without the one or more amino acid substitutions, and wherein VL1 is a λ subtype immunoglobulin light chain variable domain or a κ2 subtype immunoglobulin light chain variable domain.
35. A multivalent binding protein comprising four polypeptide chains that form three antigen binding domains; wherein the four polypeptide chains comprise:
a first heavy chain polypeptide comprising a structure represented by the formula:

VH1-L3-VH2-L4-CH1-CH2-CH3 [Ia],
a first light chain polypeptide chain comprising a structure represented by the formula:

VL2-L1-VL1-L2-CL1 [II],
a second heavy chain polypeptide comprising a structure represented by the formula:

VH3-CH1-CH2-CH3 [IIIa],
and a second light chain polypeptide chain comprising a structure represented by the formula:

VL3-CL2 [IV]
wherein:
   VL1 is a first immunoglobulin light chain variable domain;
   VL2 is a second immunoglobulin light chain variable domain;
   VL3 is a third immunoglobulin light chain variable domain;
   VH1 is a first immunoglobulin heavy chain variable domain;
   VH2 is a second immunoglobulin heavy chain variable domain;
   VH3 is a third immunoglobulin heavy chain variable domain;
   CL1 is a first immunoglobulin light chain constant domain;
   CL2 is a second immunoglobulin light chain constant domain;
   CH1 is an immunoglobulin CH1 heavy chain constant domain;
   CH2 is an immunoglobulin CH2 heavy chain constant domain;
   CH3 is an immunoglobulin CH3 heavy chain constant domain; and
   L1, L2, L3 and L4 are amino acid linkers;
   wherein the polypeptide of formula I and the polypeptide of formula II form a cross-over light chain-heavy chain pair;
   wherein VH1 and VL1 associate to form a first antigen binding domain, VH2 and VL2 associate to form a second antigen binding domain, and VH3 and VL3 associate to form a third antigen binding domain; and
   wherein:
   a) CL1 comprises one or more amino acid substitutions which reduce binding to a KappaSelect chromatography material compared to a CL1 without the one or more amino acid substitutions and VL3 comprises one or more amino acid substitutions which reduce binding to a protein L chromatography material compared to a VL3 without the one or more amino acid substitutions;
   b) CL2 comprises one or more amino acid substitutions which reduce binding to a KappaSelect chromatography material compared to a CL2 without the one or more amino acid substitutions and VL1 and VL2 each comprise one or more amino acid substitutions which reduce binding to a protein L chromatography material compared to a VL₁ and VL2 without the one or more amino acid substitutions;
   c) CL2 comprises one or more amino acid substitutions which reduce binding to a KappaSelect chromatography material compared to a CL2 without the one or more amino acid substitutions, VL1 comprises one or more amino acid substitutions which reduce binding to a protein L chromatography material compared to a VL1 without the one or more amino acid substitutions, and wherein VL2 is a λ subtype immunoglobulin light chain variable domain or a κ2 subtype light chain variable immunoglobulin domain; or
   d) CL2 comprises one or more amino acid substitutions which reduce binding to a KappaSelect chromatography material compared to a CL2 without the one or more amino acid substitutions, VL2 comprises one or more amino acid substitutions which reduce binding to a protein L chromatography material compared to a VL2 without the one or more amino acid substitutions, and wherein VL1 is a λ subtype immunoglobulin light chain variable domain or a κ2 subtype light chain variable immunoglobulin domain.
36. The multivalent binding protein of embodiment 34 or 35, wherein the binding protein is trispecific and capable of specifically binding three different antigen targets.
37. The multivalent binding protein of any one of embodiments 34-36, wherein at least one of L1, L2, L3 and/or L4 are each independently 0 amino acids in length.
38. The multivalent binding protein of any one of embodiments 34-36, wherein L1, L2, L3 and/or L4 are each independently at least one amino acid in length.
39. The multivalent binding protein of embodiment 34 or 35, wherein:
the second heavy chain polypeptide comprises a structure represented by the formula:

VH3-L5-VH4-L6-CH1 [IIIb],
and a second light chain polypeptide chain comprising a structure represented by the formula:

VL4-L7-VL3-L8-CL2 [IVa]
wherein:
   VL4 is a fourth immunoglobulin light chain variable domain;
   VH4 is a fourth immunoglobulin heavy chain variable domain;
   L5, L6, L7 and L8 are amino acid linkers;
   wherein the polypeptide of formula IIIa and the polypeptide of formula IVa form a cross-over light chain-heavy chain pair;
   wherein VH4 and VL4 associate to form a fourth antigen binding domain; and wherein:
   a) CL1 comprises one or more amino acid substitutions which reduce binding to a KappaSelect chromatography material compared to a CL1 without the one or more amino acid substitutions and VL3 and VL4 each comprise one or more amino acid substitutions which reduce binding to a protein L chromatography material compared to a VL3 and VL4 without the one or more amino acid substitutions;
   b) CL1 comprises one or more amino acid substitutions which reduce binding to a KappaSelect chromatography material compared to a CL1 without the one or more amino acid substitutions and VL3 comprises one or more amino acid substitutions which reduce binding to a protein L chromatography material compared to a VL3 without the one or more amino acid substitutions, and wherein VL4 is a λ subtype immunoglobulin light chain variable domain or a κ2 subtype immunoglobulin light chain variable domain;
   c) CL1 comprises one or more amino acid substitutions which reduce binding to a KappaSelect chromatography material compared to a CL1 without the one or more amino acid substitutions and VL4 comprises one or more amino acid substitutions which reduce binding to a protein L chromatography material compared to a VL4 without the one or more amino acid substitutions, and wherein VL3 is a λ subtype immunoglobulin light chain variable domain or a κ2 subtype immunoglobulin light chain variable domain;
   d) CL2 comprises one or more amino acid substitutions which reduce binding to a KappaSelect chromatography material compared to a CL2 without the one or more amino acid substitutions and VL1 and VL2 each comprise one or more amino acid substitutions which reduce binding to a protein L chromatography material compared to a VL1 and VL2 without the one or more amino acid substitutions;
   e) CL2 comprises one or more amino acid substitutions which reduce binding to a KappaSelect chromatography material compared to a CL2 without the one or more amino acid substitutions, VL1 comprises one or more amino acid substitutions which reduce binding to a protein L chromatography material compared to a VL1 without the one or more amino acid substitutions, and wherein VL2 is a λ subtype immunoglobulin light chain variable domain or a κ2 subtype immunoglobulin light chain variable domain; or
   f) CL2 comprises one or more amino acid substitutions which reduce binding to a KappaSelect chromatography material compared to a CL2 without the one or more amino acid substitutions, VL2 comprises one or more amino acid substitutions which reduce binding to a protein L chromatography material compared to a VL2 without the one or more amino acid substitutions, and wherein VL1 is a λ subtype immunoglobulin light chain variable domain or a κ2 subtype immunoglobulin light chain variable domain.
40. The multivalent binding protein of embodiment 39, wherein:
the second heavy chain polypeptide comprises a structure represented by the formula:

VH3-L5-VH4-L6-CH1-CH2-CH3 [IIIc],
wherein:
CH2 is an immunoglobulin CH2 heavy chain constant domain and
CH3 is an immunoglobulin CH3 heavy chain constant domain.
41. The multivalent binding protein of embodiment 39 or 40, wherein the binding protein is tetraspecific and capable of specifically binding four different antigen targets.
42. The multivalent binding protein of any one of embodiments 39-41, wherein at least one of L1, L2, L3, L4, L5, L6, L7 and/or L8 are each independently 0 amino acids in length.
43. The multivalent binding protein of any one of embodiments 39-41, wherein L1, L2, L3, L4, L5, L6, L7 and/or L8 are each independently at least one amino acid in length.
44. The multivalent binding protein of any one of embodiments 34-43, wherein binding of the CL1 or the CL2 that comprises the one or more amino acid substitutions to the KappaSelect chromatography material is reduced by about 90% compared to a CL1 or a CL2 without the one or more amino acid substitutions.
45. The multivalent binding protein of any one of embodiments 34-44, wherein binding of the VL1, the VL2, the VL3 and/or the VL4 that comprises the one or more substitutions to the protein L chromatography material is reduced by about 90% compared to a VL1, a VL2, a VL3 and/or a VL4 without the one or more amino acid substitutions.
46. The multivalent binding protein of any one of embodiments 34-45, wherein the one or more amino acid substitutions in the CL1 or the CL2 is at a position corresponding to 109, 110 or 199, wherein numbering is according to the EU index.
47. The multivalent binding protein of embodiment 46, wherein the one or more amino acid substitutions in the CL1 or the CL2 that comprises the one or more amino acid substitutions is a T109A substitution, a V110D substitution, a Q199K substitution, T109A-V110D substitutions, or T109A-V110D-Q199K substitutions, wherein amino acid numbering is according to the EU index.
48. The multivalent binding protein of any one of embodiments 34-45, wherein the one or more amino acid substitutions in the CL1 or the CL2 that comprises the one or more substitutions is at a position corresponding to 109, 198, 199, or 202, wherein numbering is according to the EU index.
49. The multivalent binding protein of embodiment 48, wherein the one or more amino acid substitutions in the CL1 or the CL2 that comprises the one or more substitutions is a H198R substitution, a Q199W substitution, or T109A-S202R substitutions, wherein amino acid numbering is according to the EU index.
50. The multivalent binding protein of any one of embodiments 34-49, wherein the one or more amino acid substitutions in the VL1, the VL2, the VL3 and/or the VL4 that comprises the one or more substitutions is a substitution of a framework amino acid.
51. The multivalent binding protein of any one of embodiments 34-50, wherein the one or more amino acid substitutions in the VL1, the VL2, the VL3 and/or the VL4 that comprises the one or more substitutions is at a position corresponding to 12 or 18, wherein numbering is according to Kabat.
52. The multivalent binding protein of embodiment 51, wherein the one or more amino acid substitutions in the VL1, the VL2, the VL3 and/or the VL4 that comprises the one or more substitutions is a S12P substitution, a R18P substitution, a R18Q substitution, S12P-R18P substitutions, or S12P-R18Q substitutions, wherein numbering is according to Kabat.
53. The multivalent binding protein of any one of embodiments 35-38 and 40-52, wherein the CH3 domain of the first heavy chain polypeptide and/or the CH3 domain of the second heavy chain polypeptide is a human IgG1 or IgG4 CH3 domain.
54. The multivalent binding protein of any one of embodiments 35-38 and 40-53, wherein the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V.
55. The multivalent binding protein of any one of embodiments 35-38 and 40-53, wherein the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W.
56. The multivalent binding protein of embodiment 54, wherein the CH3 of the second heavy chain polypeptide comprises one or more amino acid substitutions that reduce binding to protein A.
57. The multivalent binding protein of embodiment 55, wherein the CH3 of the first heavy chain polypeptide comprises one or more amino acid substitutions that reduce binding to protein A.
58. The multivalent binding protein of embodiment 56 or 57, wherein one or more amino acid substitutions that reduce binding to Protein A are amino acid substitutions at positions corresponding to positions 435 and 436 of human IgG1, wherein numbering is according to the EU Index.
59. The multivalent binding protein of embodiment 58, wherein the amino acid substitutions are H435R and Y436F, wherein amino acid numbering is according to the EU index.
60. A multivalent binding protein comprising four polypeptide chains that form two antigen binding domains; wherein the four polypeptide chains comprise:
a first heavy chain polypeptide comprising a structure represented by the formula:

VH1-CH1 [I],
a first light chain polypeptide chain comprising a structure represented by the formula:

VL1-CL1 [II],
a second heavy chain polypeptide comprising a structure represented by the formula:

VH2-CL2 [III],
and a second light chain polypeptide chain comprising a structure represented by the formula:

VL2-CH1 [IV];
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
VH1 is a first immunoglobulin heavy chain variable domain;
VH2 is a second immunoglobulin heavy chain variable domain;
CH1 is an immunoglobulin heavy chain constant domain
wherein
a) CL2 comprises one or more amino acid substitutions which reduce binding to a KappaSelect chromatography material compared to a CL2 without the one or more amino acid substitutions and VL1 comprises one or more amino acid substitutions which reduce binding to a protein L chromatography material compared to a VL1 without the one or more amino acid substitutions, or
b) CL1 comprises one or more amino acid substitutions which reduce binding to a KappaSelect chromatography material compared to a CL1 without the one or more amino acid substitutions and VL2 comprises one or more amino acid substitutions which reduce binding to a protein L chromatography material compared to a VL2 without the one or more amino acid substitutions;
and wherein VH1 and VL1 associate to form a first antigen binding domain and VH2 and VL2 associate to form a second antigen binding domain.
61. A multivalent binding protein comprising four polypeptide chains that form two antigen binding domains; wherein the four polypeptide chains comprise:
a first heavy chain polypeptide comprising a structure represented by the formula:

VH1-CH1-CH2-CH3 [I],
a first light chain polypeptide chain comprising a structure represented by the formula:

VL1-CL1 [II],
a second heavy chain polypeptide comprising a structure represented by the formula:

VH2-CL2-CH2-CH3 [III],
and a second light chain polypeptide chain comprising a structure represented by the formula:

VL2-CH1 [IV];
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
VH1 is a first immunoglobulin heavy chain variable domain;
VH2 is a second immunoglobulin heavy chain variable domain;
CH1 is an immunoglobulin heavy chain constant domain;
CH2 is an immunoglobulin CH2 heavy chain constant domain; and
CH3 is an immunoglobulin CH3 heavy chain constant domain
wherein
   a) CL2 comprises one or more amino acid substitutions which reduce binding to a KappaSelect chromatography material compared to a CL2 without the one or more amino acid substitutions and VL1 comprises one or more amino acid substitutions which reduce binding to a protein L chromatography material compared to a VL1 without the one or more amino acid substitutions, or
   b) CL1 comprises one or more amino acid substitutions which reduce binding to a KappaSelect chromatography material compared to a CL1 without the one or more amino acid substitutions and VL2 comprises one or more amino acid substitutions which reduce binding to a protein L chromatography material compared to a VL2 without the one or more amino acid substitutions;
and wherein VH1 and VL1 associate to form a first antigen binding domain and VH2 and VL2 associate to form a second antigen binding domain.
62. A multivalent binding protein comprising four polypeptide chains that form two antigen binding domains; wherein the four polypeptide chains comprise:
a first heavy chain polypeptide comprising a structure represented by the formula:

VH1-CH1 [I],
a first light chain polypeptide chain comprising a structure represented by the formula:

VL1-CL1 [II],
a second heavy chain polypeptide comprising a structure represented by the formula:

VL₂-CH₁ [III],
and a second light chain polypeptide chain comprising a structure represented by the formula:

VH2-CL2 [IV];
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
VH1 is a first immunoglobulin heavy chain variable domain;
VH2 is a second immunoglobulin heavy chain variable domain;
CH1 is an immunoglobulin heavy chain constant domain;
CH2 is an immunoglobulin CH2 heavy chain constant domain; and
CH3 is an immunoglobulin CH3 heavy chain constant domain;
wherein
   a) CL2 comprises one or more amino acid substitutions which reduce binding to a KappaSelect chromatography material compared to a CL2 without the one or more amino acid substitutions and VL1 comprises one or more amino acid substitutions which reduce binding to a protein L chromatography material compared to a VL1 without the one or more amino acid substitutions, or
   b) CL1 comprises one or more amino acid substitutions which reduce binding to a KappaSelect chromatography material compared to a CL1 without the one or more amino acid substitutions and VL2 comprises one or more amino acid substitutions which reduce binding to a protein L chromatography material compared to a VL2 without the one or more amino acid substitutions;
and wherein VH1 and VL1 associate to form a first antigen binding domain and VH2 and VL2 associate to form a second antigen binding domain.
63. A multivalent binding protein comprising four polypeptide chains that form two antigen binding domains; wherein the four polypeptide chains comprise:
a first heavy chain polypeptide comprising a structure represented by the formula:

VH1-CH1-CH2-CH3 [I],
a first light chain polypeptide chain comprising a structure represented by the formula:

VL1-CL1 [II],
a second heavy chain polypeptide comprising a structure represented by the formula:

VH2-CH1-CH2-CH3 [III],
and a second light chain polypeptide chain comprising a structure represented by the formula:

VL2-CL2 [IV];
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
VH1 is a first immunoglobulin heavy chain variable domain;
VH2 is a second immunoglobulin heavy chain variable domain;
CH1 is an immunoglobulin heavy chain constant domain;
CH2 is an immunoglobulin CH2 heavy chain constant domain; and
CH3 is an immunoglobulin CH3 heavy chain constant domain;
wherein
   a) CL2 comprises one or more amino acid substitutions which reduce binding to a KappaSelect chromatography material compared to a CL2 without the one or more amino acid substitutions and VL1 comprises one or more amino acid substitutions which reduce binding to a protein L chromatography material compared to a VL1 without the one or more amino acid substitutions, or
   b) CL1 comprises one or more amino acid substitutions which reduce binding to a KappaSelect chromatography material compared to a CL1 without the one or more amino acid substitutions and VL2 comprises one or more amino acid substitutions which reduce binding to a protein L chromatography material compared to a VL2 without the one or more amino acid substitutions;
and wherein VH1 and VL1 associate to form a first antigen binding domain and VH2 and VL2 associate to form a second antigen binding domain.
64. The multivalent binding protein of any one of embodiments 60-63, wherein binding of the CL1 or the CL2 that comprises the one or more amino acid substitutions to the KappaSelect chromatography material is reduced by about 90% compared to a CL1 or a CL2 without the one or more amino acid substitutions.
65. The multivalent binding protein of any one of embodiments 60-64, wherein binding of the VL1 or the VL2 that comprises the one or more amino acid substitutions to the protein L chromatography material is reduced by about 90% compared to a VL1 or a VL2 without the one or more amino acid substitutions.
66. The multivalent binding protein of any one of embodiments 60-65, wherein the one or more amino acid substitutions in the CL1 or the CL2 that comprises the one or more amino acid substitutions is at a position corresponding to 109, 110 or 199, wherein numbering is according to the EU index.
67. The multivalent binding protein of embodiment 66, wherein the one or more amino acid substitutions in the CL1 or the CL2 that comprises the one or more amino acid substitutions is a T109A substitution, a V110D substitution, a Q199K substitution, T109A-V110D substitutions, or T109A-V110D-Q199K substitutions.
68. The multivalent binding protein of any one of embodiments 60-65, wherein the one or more amino acid substitutions in the CL1 or the CL2 that comprises the one or more substitutions is at a position corresponding to 109, 198, 199, or 202, wherein numbering is according to the EU index.
69. The multivalent binding protein of embodiment 68, wherein the one or more amino acid substitutions in the CL1 or the CL2 that comprises the one or more substitutions is a H198R substitution, a Q199W substitution, or T109A-S202R substitutions, wherein amino acid numbering is according to the EU index.
70. The multivalent binding protein of any one of embodiments 60-69, wherein the one or more amino acid substitutions in the VL1 or the VL2 that comprises the one or more amino acid substitutions is a substitution of a framework amino acid.
71. The multivalent binding protein of any one of embodiments 60-70, wherein the one or more amino acid substitutions in the VL1 or the VL2 that comprises the one or more amino acid substitutions is at a position corresponding to 12 or 18, wherein numbering is according to Kabat.
72. The multivalent binding protein of embodiment 71, wherein the one or more amino acid substitutions in VL1 or VL2 that comprises the one or more amino acid substitutions is a S12P substitution, a R18P substitution, a R18Q substitution, S12P-R18P substitutions, or S12P-R18Q substitutions, wherein numbering is according Kabat.
73. The multivalent binding protein of any one of embodiments 57-66, wherein the CH3 domain of the first heavy chain polypeptide and/or the CH3 domain of the second heavy chain polypeptide is a human IgG1 or IgG4 CH3 domain.
74. The multivalent binding protein of any one of embodiments 61-73, wherein the CH₃ domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V.
75. The multivalent binding protein of any one of embodiments 61-74, wherein the CH₃ domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W.
76. The multivalent binding protein of embodiment 74, wherein the CH3 of the second heavy chain polypeptide comprises one or more amino acid substitutions that reduce binding to a Protein A chromatography material.
77. The multivalent binding protein of embodiment 75, wherein the CH3 of the first heavy chain polypeptide comprises one or more amino acid substitutions that reduce binding to a Protein A chromatography material.
78. The multivalent binding protein of embodiment 76 or 77, wherein one or more amino acid substitutions which reduces binding to the Protein A chromatography material are amino acid substitutions at positions corresponding to positions 435 and 436 of human IgG1, wherein numbering is according to the EU Index.
79. The multivalent binding protein of embodiment 78, wherein the amino acid substitutions are H435R and Y436F, wherein amino acid numbering is according to the EU index.
80. The multivalent binding protein of any one of embodiments 56-73, wherein the multivalent binding protein is a bispecific antigen binding protein.
81. The multivalent binding protein of any one of embodiments 60-80, wherein the first antigen binding domain and the second antigen binding domain bind different antigens.
82. A multivalent binding protein comprising four polypeptide chains that form four antigen binding domains; wherein the four polypeptide chains comprise:
a first heavy chain polypeptide comprising a structure represented by the formula:

VH1-CH11-L1-VH2-CH12 [I],
a first light chain polypeptide comprising a structure represented by the formula:

VL1-CL1-L2-VL2-CL2 [II],
a second heavy chain polypeptide comprising a structure represented by the formula:

VH3-CH13-L3-VH4-CH14 [III],
a second light chain polypeptide comprising a structure represented by the formula:

VL3-CL3-L4-VL4-CL4 [IV]
wherein:
   VL1 is a first immunoglobulin light chain variable domain;
   VL2 is a second immunoglobulin light chain variable domain;
   VL3 is a third immunoglobulin light chain variable domain;
   VL4 is a fourth immunoglobulin light chain variable domain;
   CL1 is a first immunoglobulin light chain constant domain;
   CL2 is a second immunoglobulin light chain constant domain;
   CL3 is a third immunoglobulin light chain constant domain;
   CL4 is a fourth immunoglobulin light chain constant domain;
   VH1 is a first immunoglobulin heavy chain variable domain;
   VH2 is a second immunoglobulin heavy chain variable domain;
   VH3 is a third immunoglobulin heavy chain variable domain;
   VH4 is a fourth immunoglobulin heavy chain variable domain;
   CH11 is a first immunoglobulin heavy chain constant domain;
   CH12 is a second immunoglobulin heavy chain constant domain;
   CH13 is a third immunoglobulin heavy chain constant domain;
   CH14 is a fourth immunoglobulin heavy chain constant domain;
   and
   L1, L2, L3 and L4 are amino acid linkers;
   wherein:
   a) CL1 and CL2 each comprise one or more amino acid substitutions which reduce binding to a KappaSelect chromatography material compared to a CL1 and CL2 without the one or more amino acid substitutions and VL3 and VL4 each comprise one or more amino acid substitutions which reduce binding to a protein L chromatography material compared to a VL3 and VL4 without the one or more amino acid substitutions;
   b) CL1 and CL2 each comprise one or more amino acid substitutions which reduce binding to a KappaSelect chromatography material compared to a CL1 and CL2 without the one or more amino acid substitutions, VL3 comprises one or more amino acid substitutions which reduce binding to a protein L chromatography material compared to a VL3 without the one or more amino acid substitutions, and VL₄ is a λ subtype immunoglobulin light chain variable domain or a κ2 subtype immunoglobulin light chain variable domain;
   c) CL1 and CL2 each comprises one or more amino acid substitutions which reduce binding to a KappaSelect chromatography material compared to a CL1 and CL2 without the one or more amino acid substitutions, VL4 comprises one or more amino acid substitutions which reduce binding to a protein L chromatography material compared to a VL4 without the one or more amino acid substitutions, and wherein VL3 is a λ subtype immunoglobulin light chain variable domain or a κ2 subtype immunoglobulin light chain variable domain;
   d) CL1 comprises one or more amino acid substitutions which reduce binding to a KappaSelect chromatography material compared to a CL1 without the one or more amino acid substitutions, CL2 is a λ subtype immunoglobulin light chain constant domain, and VL3 and VL4 each comprise one or more amino acid substitutions which reduce binding to a protein L chromatography material compared to a VL₁ and VL2 without the one or more amino acid substitutions;
   e) CL1 comprises one or more amino acid substitutions which reduce binding to a KappaSelect chromatography material compared to a CL1 without the one or more amino acid substitutions, CL2 is a λ subtype immunoglobulin light chain constant domain, VL3 comprises one or more amino acid substitutions which reduce binding to a protein L chromatography material compared to a VL3 without the one or more amino acid substitutions, and VL4 is a λ subtype immunoglobulin light chain variable domain or a κ2 subtype immunoglobulin light chain variable domain;
   f) CL1 comprises one or more amino acid substitutions which reduce binding to a KappaSelect chromatography material compared to a CL1 without the one or more amino acid substitutions, CL2 is a λ subtype immunoglobulin light chain constant domain, VL4 comprises one or more amino acid substitutions which reduce binding to a protein L chromatography material compared to a VL4 without the one or more amino acid substitutions, and wherein VL3 is a λ subtype immunoglobulin light chain variable domain or a κ2 subtype immunoglobulin light chain variable domain;
   g) CL2 comprises one or more amino acid substitutions which reduce binding to a KappaSelect chromatography material compared to a CL2 without the one or more amino acid substitutions, CL1 is a λ subtype immunoglobulin light chain constant domain, and VL3 and VL4 each comprise one or more amino acid substitutions which reduce binding to a protein L chromatography material compared to a VL3 and VL4 without the one or more amino acid substitutions;
   h) CL2 comprises one or more amino acid substitutions which reduce binding to a KappaSelect chromatography material compared to a CL2 without the one or more amino acid substitutions, CL1 is a λ subtype immunoglobulin light chain constant domain, VL3 comprises one or more amino acid substitutions which reduce binding to a protein L chromatography material compared to a VL3 without the one or more amino acid substitutions, and VL4 is a λ subtype immunoglobulin light chain variable domain or a κ2 subtype immunoglobulin light chain variable domain;
   i) CL2 comprises one or more amino acid substitutions which reduce binding to a KappaSelect chromatography material compared to a CL2 without the one or more amino acid substitutions, CL1 is a λ subtype immunoglobulin light chain constant domain, VL4 comprises one or more amino acid substitutions which reduce binding to a protein L chromatography material compared to a VL4 without the one or more amino acid substitutions, and wherein VL3 is a λ subtype immunoglobulin light chain variable domain or a κ2 subtype immunoglobulin light chain variable domain;
and wherein VL1 and VH1 form a first antigen binding domain, VL2 and VH2 form a second antigen binding domain, VL3 and VH3 form a third antigen binding domain, and VL4 and VH4 form a fourth antigen binding domain.
83. The multivalent binding protein of embodiment 82, wherein at least one of L1, L2, L3 or L4 are each independently 0 amino acids in length.
84. The binding protein of embodiment 82 or 83, wherein at least one of L1, L2, L3 or L4 are each independently at least one amino acid in length.
85. A multivalent binding protein comprising four polypeptide chains that form four antigen binding domains; wherein the four polypeptide chains comprise:
a first heavy chain polypeptide comprising a structure represented by the formula:

VH1-L1-VH2-L2-CH11 [I],
a first light chain polypeptide comprising a structure represented by the formula:

VL1-L3-VL2-L4-CL1 [II],
a second heavy chain polypeptide comprising a structure represented by the formula:

VH3-L5-VH4-L6-CH12 [III],
a second light chain polypeptide comprising a structure represented by the formula:

VL3-L7-VL4-L8-CL2 [IV]
wherein:
   VL1 is a first immunoglobulin light chain variable domain;
   VL2 is a second immunoglobulin light chain variable domain;
   VL3 is a third immunoglobulin light chain variable domain;
   VL4 is a fourth immunoglobulin light chain variable domain;
   CL1 is a first immunoglobulin light chain constant domain;
   CL2 is a second immunoglobulin light chain constant domain;
   VH1 is a first immunoglobulin heavy chain variable domain;
   VH2 is a second immunoglobulin heavy chain variable domain;
   VH3 is a third immunoglobulin heavy chain variable domain;
   VH4 is a fourth immunoglobulin heavy chain variable domain;
   CH11 is a first immunoglobulin heavy chain constant domain;
   CH12 is a second immunoglobulin heavy chain constant domain;
   and
   L1, L2, L3, L4 L5, L6, L7 and L8 are amino acid linkers;
   wherein:
   a) CL1 comprises one or more amino acid substitutions which reduce binding to a KappaSelect chromatography material compared to a CL1 without the one or more amino acid substitutions and VL3 and VL4 each comprise one or more amino acid substitutions which reduce binding to a protein L chromatography material compared to a VL3 and VL4 without the one or more amino acid substitutions;
   b) CL1 comprises one or more amino acid substitutions which reduce binding to a KappaSelect chromatography material compared to a CL1 without the one or more amino acid substitutions, VL3 comprises one or more amino acid substitutions which reduce binding to a protein L chromatography material compared to a VL3 without the one or more amino acid substitutions, and VL4 is a λ subtype immunoglobulin light chain variable domain or a λ2 subtype immunoglobulin light chain variable domain;
   c) CL1 comprises one or more amino acid substitutions which reduce binding to a KappaSelect chromatography material compared to a CL1 without the one or more amino acid substitutions, VL4 comprises one or more amino acid substitutions which reduce binding to a protein L chromatography material compared to a VL4 without the one or more amino acid substitutions, and wherein VL3 is a λ subtype immunoglobulin light chain variable domain or a κ2 subtype immunoglobulin light chain variable domain;
and wherein VL1 and VH1 form a first antigen binding domain, VL2 and VH2 form a second antigen binding domain, VL3 and VH3 form a third antigen binding domain, and VL4 and VH4 form a fourth antigen binding domain.
86. The multivalent binding protein of embodiment 85, wherein at least one of L1, L2, L3, L4, L5, L6, L7, or L8 are each independently 0 amino acids in length.
87. The binding protein of embodiment 85 or 86, wherein at least one of L1, L2, L3, L4, L5, L6, L7, or L8 are each independently at least one amino acid in length.
88. The multivalent binding protein of any one of embodiments 82-87, wherein binding of the CL1 and/or the CL2 that comprises the one or more amino acid substitutions to the KappaSelect chromatography material is reduced by about 90% compared to a CL1 and/or a CL2 without the one or more amino acid substitutions.
89. The multivalent binding protein of any one of embodiments 82-88, wherein binding of the VL3 and/or the VL4 that comprises the one or more amino acid substitutions to the protein L chromatography material is reduced by about 90% compared to a VL3 and/or a VL4 without the one or more amino acid substitutions.
90. The multivalent binding protein of any one of embodiments 82-89, wherein the one or more amino acid substitutions in the CL₁ and/or the CL₂ that comprises the one or more amino acid substitutions is at a position corresponding to 109, 110 or 199, wherein numbering is according to the EU index.
91. The multivalent binding protein of embodiment 90, wherein the one or more amino acid substitutions in the CL1 and/or the CL2 that comprises the one or more amino acid substitutions is a T109A substitution, a V110D substitution, a Q199K substitution, T109A-V110D substitutions, or T 109A-V 110D-Q199K substitutions, wherein amino acid numbering is according to the EU index.
92. The multivalent binding protein of any one of embodiments 82-89, wherein the one or more amino acid substitutions in the CL1 and/or the CL2 that comprises the one or more substitutions is at a position corresponding to 109, 198, 199, or 202, wherein numbering is according to the EU index.
93. The multivalent binding protein of embodiment 92, wherein the one or more amino acid substitutions in the CL1 and/or the CL2 that comprises the one or more substitutions is a H198R substitution, a Q199W substitution, or T109A-S202R substitutions, wherein amino acid numbering is according to the EU index.
94. The multivalent binding protein of any one of embodiments 82-93, wherein the one or more amino acid substitutions in the VL3 and/or the VL4 that comprises the one or more amino acid substitutions is a substitution of a framework amino acid.
95. The multivalent binding protein of any one of embodiments 82-94, wherein the one or more amino acid substitutions in the VL3 and/or the VL4 that comprises the one or more amino acid substitutions is at a position corresponding to 12 or 18, wherein numbering is according to Kabat.
96. The multivalent binding protein of embodiment 95, wherein the one or more amino acid substitutions in the VL3 and/or the VL4 that comprises the one or more amino acid substitutions is a S12P substitution, a R18P substitution, a R18Q substitution, S12P-R18P substitutions, or S12P-R18Q substitutions, wherein numbering is according to Kabat.
97. The multivalent binding protein of any one of embodiments 82 or 86-96, wherein
the first heavy chain polypeptide comprising a structure represented by the formula:

VH1-CH11-L1-VH2-CH12-CH2-CH3 [Ia],
the second heavy chain polypeptide comprising a structure represented by the formula:

VH3-CH13-L3-VH4-CH14-CH2-CH3 [IIIa].
98. The multivalent binding protein of any one of embodiments 83-96, wherein
the first heavy chain polypeptide comprising a structure represented by the formula:

VH1-L1-VH2-L2-CH11-CH2-CH3 [Ia],
the second heavy chain polypeptide comprising a structure represented by the formula:

VH3-L5-VH4-L6-CH12-CH2-CH3 [IIIa].
99. The multivalent binding protein embodiment 97, wherein the CH3 domain of the first heavy chain polypeptide and/or the CH3 domain of the second heavy chain polypeptide is a human IgG1 or IgG4 CH3 domain.
100. The multivalent binding protein of embodiment 97 or 99, wherein the CH3 domain of the first heavy chain polypeptide comprises amino acid substitutions at positions corresponding to positions 354 and 366 of human IgG1, wherein numbering is according to the EU Index, wherein the amino acid substitutions are S354C and T366W; wherein the CH3 domain of the second heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V.
101. The multivalent binding protein of any one of embodiments 96-100, wherein the CH3 domain of the first heavy chain polypeptide comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V; wherein the CH3 domain of the second heavy chain polypeptide comprises amino acid substitutions at positions corresponding to positions 354 and 366 of human IgG1, wherein numbering is according to the EU Index, wherein the amino acid substitutions are S354C and T366W.
102. The multivalent binding protein of embodiment 101, wherein the CH3 of the second heavy chain polypeptide comprises one or more amino acid substitutions that reduce binding to a Protein A chromatography material.
103. The multivalent binding protein of embodiment 101, wherein the CH3 of the first heavy chain polypeptide comprises one or more amino acid substitutions that reduce binding to a Protein A chromatography material.
104. The multivalent binding protein of embodiment 102 or 103, wherein one or more amino acid substitutions that reduce binding to the Protein A chromatography material are amino acid substitutions at positions corresponding to positions 435 and 436 of human IgG1, wherein numbering is according to the EU Index.
105. The multivalent binding protein of embodiment 104, wherein the amino acid substitutions are H435R and Y436F, wherein amino acid numbering is according to the EU index.
106. The multivalent binding protein of any one of embodiments 82-105, wherein the binding protein is tetraspecific and capable of specifically binding four different antigen targets.
107. A multivalent binding protein comprising four polypeptide chains that form an antigen binding domain; wherein the four polypeptide chains comprise:
a first heavy chain polypeptide comprises a structure represented by the formula:

VH1-CH11 [I],
a first light chain polypeptide chain comprises a structure represented by the formula:

VL1-CL1 [II],
a second heavy chain polypeptide comprises a structure represented by the formula:

fusion polypeptide-L1-CH12 [III],
and a second light chain polypeptide chain comprises a structure represented by the formula:

fusion polypeptide-L2-CL2 [IV]
wherein:
VL1 is a first immunoglobulin light chain variable domain;
CL1 is a first immunoglobulin light chain constant domain;
CL2 is a second immunoglobulin light chain constant domain;
VH1 is a first immunoglobulin heavy chain variable domain;
CH11 is a first immunoglobulin heavy chain constant domain;
CH12 is a second immunoglobulin heavy chain constant domain; and
L1 and L2 are amino acid linkers;
wherein CL2 comprises one or more amino acid substitutions which reduce binding to a KappaSelect chromatography material compared to a CL2 without the one or more amino acid substitutions and VL1 comprises one or more amino acid substitutions which reduce binding to a protein L chromatography material compared to a VL1 without the one or more amino acid substitutions; and
wherein VL1 and VH1 form an antigen binding domain.
108. The multivalent binding protein of embodiment 107, wherein L1 or L2 is independently 0 amino acids in length.
109. The binding protein of embodiment 107 or 108, wherein L1 or L2 is independently at least one amino acid in length.
110. A multivalent binding protein comprising four polypeptide chains that form two antigen binding domains; wherein the four polypeptide chains comprise:
a first heavy chain polypeptide comprises a structure represented by the formula:

VH1-CH11-L1-VH2-CH12 [I],
a first light chain polypeptide chain comprises a structure represented by the formula:

VL1-CL1-L2-VL2-CL2 [II],
a second heavy chain polypeptide comprises a structure represented by the formula:

fusion polypeptide-L3-CH13 [III],
and a second light chain polypeptide comprises a structure represented by the formula:

fusion polypeptide-L4-CL3 [IV]
wherein:
VL1 is a first immunoglobulin light chain variable domain;
VL2 is a second immunoglobulin light chain variable domain;
CL1 is a first immunoglobulin light chain constant domain;
CL2 is a second immunoglobulin light chain constant domain;
CL3 is a third immunoglobulin light chain constant domain;
VH1 is a first immunoglobulin heavy chain variable domain;
VH2 is a second immunoglobulin heavy chain variable domain;
CH11 is a first immunoglobulin heavy chain constant domain;
CH12 is a second immunoglobulin heavy chain constant domain;
CH13 is a third immunoglobulin heavy chain constant domain, and
L1, L2, L3 and L4 are amino acid linkers;
wherein:
a) CL3 comprises one or more amino acid substitutions which reduce binding to a KappaSelect chromatography material compared to a CL3 without the one or more amino acid substitutions, VL1 and VL2 each comprise one or more amino acid substitutions which reduce binding to a protein L chromatography material compared to a VL1 and VL2 without the one or more amino acid substitutions,
b) CL3 comprises one or more amino acid substitutions which reduce binding to a KappaSelect chromatography material compared to a CL3 without the one or more amino acid substitutions, VL1 comprises one or more amino acid substitutions which reduce binding to a protein L chromatography material compared to a VL1 without the one or more amino acid substitutions, and VL2 is a λ subtype immunoglobulin light chain variable domain or a λ2 subtype immunoglobulin domain; or
c) CL3 comprises one or more amino acid substitutions which reduce binding to a KappaSelect chromatography material compared to a CL3 without the one or more amino acid substitutions, VL2 comprises one or more amino acid substitutions which reduce binding to a protein L chromatography material compared to a VL2 without the one or more amino acid substitutions, and VL1 is a λ subtype immunoglobulin light chain variable domain or a λ₂ subtype immunoglobulin domain; and
wherein VL1 and VH1 form a first antigen binding domain and VL2 and VH2 form a second antigen binding domain.
111. The multivalent binding protein of embodiment 110, wherein at least one of L1, L2, L3, or L4 are each independently 0 amino acids in length.
112. The binding protein of embodiment 110 or 111, wherein L1, L2, L3 or L4 are each independently at least one amino acid in length.
113. The multivalent binding protein of any one of embodiments 99-104, wherein binding of the CL2 or the CL3 that comprise the one or more amino acid substitutions to the KappaSelect chromatography material is reduced by about 90% compared to a CL2 or a CL3 without the one or more amino acid substitutions.
114. The multivalent binding protein of any one of embodiments 107-113, wherein binding of the VL1 and/or the VL2 that comprise the one or more amino acid substitutions to the protein L chromatography material is reduced by about 90% compared to a VL1 and/or a VL2 without the one or more amino acid substitutions.
115. The multivalent binding protein of any one of embodiments 107-114, wherein the one or more amino acid substitutions in the CL2 or the CL3 that comprise the one or more amino acid substitutions is at a position corresponding to 109, 110 or 199, wherein numbering is according to the EU index.
116. The multivalent binding protein of embodiment 115, wherein the one or more amino acid substitutions in the CL2 or the CL3 that comprise the one or more amino acid substitutions is a T109A substitution, a V110D substitution, a Q199K substitution, T109A-V110D substitutions, or T109A-V110D-Q199K substitutions.
117. The multivalent binding protein of any one of embodiments 107-114, wherein the one or more amino acid substitutions in the CL2 or the CL3 that comprises the one or more substitutions is at a position corresponding to 109, 198, 199, or 202, wherein numbering is according to the EU index.
118. The multivalent binding protein of embodiment 117, wherein the one or more amino acid substitutions in the CL2 or the CL3 that comprises the one or more substitutions is a H198R substitution, a Q199W substitution, or T109A-S202R substitutions, wherein amino acid numbering is according to the EU index.
119. The multivalent binding protein of any one of embodiments 107-118, wherein the one or more amino acid substitutions in the VL1 and/or the VL2 that comprise the one or more amino acid substitutions is a substitution of a framework amino acid.
120. The multivalent binding protein of any one of embodiments 107-119, wherein the one or more amino acid substitutions in the VL1 and/or the VL2 that comprise the one or more amino acid substitutions is at a position corresponding to 12 or 18, wherein numbering is according to Kabat.
121. The multivalent binding protein of embodiment 120, wherein the one or more amino acid substitutions in the VL1 and/or the VL2 is a S12P substitution, a R18P substitution, a R18Q substitution, S12P-R18P substitutions, or S12P-R18Q substitutions, wherein numbering is according to Kabat.
122. The multivalent binding protein of any one of embodiments 107-121, wherein the first heavy chain polypeptide comprises a first CH2 immunoglobulin heavy chain constant domain and a first CH3 immunoglobulin heavy chain constant domain and the second heavy chain polypeptide comprises a second CH2 immunoglobulin heavy chain constant domain and a second CH3 immunoglobulin heavy chain constant domain.
123. The multivalent binding protein of embodiment 122, wherein the first CH3 domain and/or the CH3 domain is a human IgG1 or IgG4 CH3 domain.
124. The multivalent binding protein of any one of embodiments 122 or 123, wherein the first CH3 domain comprises amino acid substitutions at positions corresponding to positions 354 and 366 of human IgG1, wherein numbering is according to the EU Index, wherein the amino acid substitutions are S354C and T366W; wherein second CH3 domain comprises amino acid substitutions at positions corresponding to positions 349, 366, 368, 407, 435, and 436 of human IgG1, wherein numbering is according to the EU Index, wherein the amino acid substitutions are Y349C, T366S, L368A, and Y407V.
125. The multivalent binding protein of embodiment 124, wherein the CH3 of the second heavy chain polypeptide comprises one or more amino acid substitutions which reduces binding to protein A.
126. The multivalent binding protein of embodiment 125, wherein the CH3 of the first heavy chain polypeptide comprises one or more amino acid substitutions which reduces binding to protein A.
127. The multivalent binding protein of embodiment 126, wherein one or more amino acid substitutions which reduces binding to Protein A are amino acid substitutions at positions corresponding to positions 435 and 436 of human IgG1, wherein numbering is according to the EU Index.
128. The multivalent binding protein of embodiment 127, wherein the amino acid substitutions are H435R and Y436F, wherein amino acid numbering is according to the EU index.
129. The multivalent binding protein of any one of embodiments 1-128, wherein the multivalent binding protein is a multispecific antibody or antigen binding fragment thereof.
130. One or more polynucleotide(s) encoding the multivalent binding protein of any one of embodiments 1-129.
131. One or more vector(s) comprising the one or more polynucleotide(s) of embodiment 130.
132. A host cell comprising the one or more polynucleotide(s) of embodiment 130, or the one or more vector(s) of embodiment 131.
133. A method of producing a multivalent binding protein, the method comprising culturing the host cell of embodiment 122 such that the binding protein is produced.
134. The method of embodiment 133, further comprising recovering the binding protein from the host cell.
135. A pharmaceutical composition comprising the multivalent binding protein of any one of embodiments 1-129 and a pharmaceutically acceptable carrier.
136. A method of purifying the multivalent binding protein of any one of embodiments 1 to 129, the method comprising
a) subjecting a composition comprising the multivalent binding protein to Protein L chromatography in bind and elute mode to generate a protein L eluate, and
b) subjecting the protein L eluate to KappaSelect chromatography in bind and elute mode to generate a KappaSelect eluate,
wherein the KappaSelect eluate comprises the multivalent binding protein and is essentially free of mispaired polypeptides.
137. The method of embodiment 136, wherein the multivalent binding protein in the KappaSelect eluate is at least 85% pure, at least 90% pure, or at least 95% pure.
138. The method of embodiment 136 or 137, wherein less than 15%, less than 10%, or less than 5% of the polypeptides in the KappaSelect eluate are mispaired polypeptides.
139. A method of purifying the multivalent binding protein of any one of embodiments 1 to 129, the method comprising
a) subjecting a composition comprising the multivalent binding protein and mispaired antibodies to KappaSelect chromatography in bind and elute chromatography to generate as KappaSelect eluate and
b) subjecting the KappaSelect eluate to Protein L chromatography in bind and elute mode to generate a protein L eluate,
wherein the protein L eluate comprises the multivalent binding protein and is essentially free of mispaired polypeptides.
140. The method of embodiment 139, wherein the multivalent binding protein in the protein L eluate is at least 85% pure, at least 90% pure, or at least 95% pure.
141. The method of embodiment 139 or 140, wherein less than 15%, less than 10%, or less than 5% of the polypeptides in the protein L eluate are mispaired polypeptides.
142. A method of purifying the multivalent binding protein of any one of embodiments 1 to 129, the method comprising
a) subjecting a composition comprising the multivalent binding protein to Protein A chromatography in bind and elute mode to generate a Protein A eluate,
b) subjecting the Protein A eluate to Protein L chromatography in bind and elute mode to generate a protein L eluate, and
c) subjecting the protein L eluate to KappaSelect chromatography in bind and elute mode to generate a KappaSelect eluate,
wherein the KappaSelect eluate comprises the multivalent binding protein and is essentially free of mispaired polypeptides.
143. The method of embodiment 142, wherein the multivalent binding protein in the KappaSelect eluate is at least 85% pure, at least 90% pure, or at least 95% pure.
144. The method of embodiment 142 or 143, wherein less than 15%, less than 10%, or less than 5% of the polypeptides in the KappaSelect eluate are mispaired polypeptides.
145. A method of purifying the multivalent binding protein of any one of embodiments 1 to 129, the method comprising
a) subjecting a composition comprising the multivalent binding protein to Protein A chromatography in bind and elute mode to generate a Protein A eluate,
b) subjecting the Protein A eluate to KappaSelect chromatography in bind and elute mode to generate a KappaSelect eluate, and
c) subjecting the protein KappaSelect eluate to Protein L chromatography in bind and elute mode to generate a protein L eluate,
wherein the L eluate comprises the multivalent binding protein and is essentially free of mispaired polypeptides.
146. The method of embodiment 145, wherein the multivalent binding protein in the protein L eluate is at least 85% pure, at least 90% pure, or at least 95% pure.
147. The method of embodiment 145 or 146, wherein less than 15%, less than 10%, or less than 5% of the polypeptides in the protein L eluate are mispaired polypeptides.
148. The method of any one of embodiments 136-147, wherein the composition comprising the multivalent binding protein is derived from a host cell engineered to express the multispecific binding protein.
149. The method of any one of embodiments 136-148, wherein the composition comprising the multivalent binding protein is a host cell culture supernatant.
150. The method of any one of embodiments 136-149, wherein the composition comprising the multivalent binding protein further comprises mispaired polypeptides.
151. The method of any one of embodiments 136-150, wherein the composition comprising the multivalent binding protein is filtered prior to chromatography.
152. The method of any one of embodiments 136-151, further comprising a polishing step after the KappaSelect or protein L chromatography.
153. The method of embodiment 152, wherein the polishing step is a size exclusion chromatography.
154. The method of any one of embodiments 146-153, wherein the Protein A chromatography is a MabSelect™, MabSelect SuRe™, MabSelect SuRe™ LX, MabSelect PrismA, ProSep®-vA, ProSep® Ultra Plus, Protein A Sepharose® Fast Flow, or Toyopearl® AF-rProtein A chromatography.
155. The method of any one of embodiments 136-154, wherein the protein L chromatography is a Pierce™ Protein L chromatography cartridge, a Capto™ L chromatography, HiTrap® Protein L chromatography, a TOYOPEARL® AF-rProtein L-650F chromatography, or a KanCap™ L chromatography.
156. The method of any one of embodiments 136-155, wherein the KappaSelect chromatography is a HiTrap™ KappaSelect or a CaptureSelect™ Kappa XL chromatography.
157. The method of any one of embodiments 136-156, wherein the composition comprising the multivalent binding protein is combined with a pharmaceutically acceptable carrier.

### Examples

The Examples that follow are illustrative of specific embodiments of the disclosure, and various uses thereof. They are set forth for explanatory purposes only, and should not be construed as limiting the scope of the invention in any way.

### Example 1. Development of Pro-L KO Mutations

Peptostreptococcus magnus protein L (PpL) is a multidomain, bacterial surface protein that interacts with the variable light chain (VL) region of subclasses of human antibody kappa light chains. It does not interact with human antibody lambda light chains. As such, recombinant forms of protein-L have a breadth of applications for the purification of different antibodies and antibody fragments when they are immobilized to chromatography matrices.

To enable the development of novel purification approaches mutations that would diminish or completely abrogate the binding of an antibody light chain (LC) to protein-L were identified. A series of single, double, or triple amino acid substitutions and/or deletions were introduced in the variable light chain framework-1 (FW1) domain of adalimumab, a well characterized antibody targeting TNFα. Adalimumab variants containing these different mutant LCs were produced and characterized these relative to the wild type version for protein-L ligand binding in solution or immobilized on resin, as well as several other qualities including expression titer, interaction with the target protein TNFα, accelerated stability, and predicted immunogenicity.

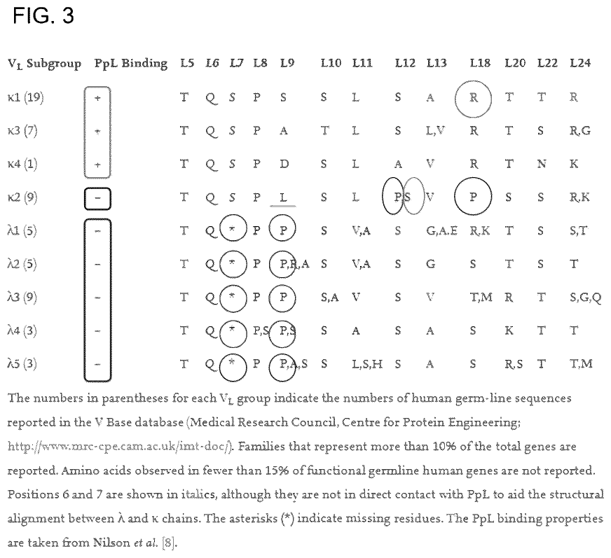

FIG. 3 shows the alignment of residues in human VL kappa and lambda subtypes and their PpL binding properties. Residues most observed in the germline of these genes are shown for each subtype. The sequence information, as well as structural data reported on the nature of the interaction of protein-L with the VL region of antibodies, was used to design a series of mutations in the VL FW1 of our test molecule, Adalimumab to identify changes that would diminish or completely abolish protein-L binding.

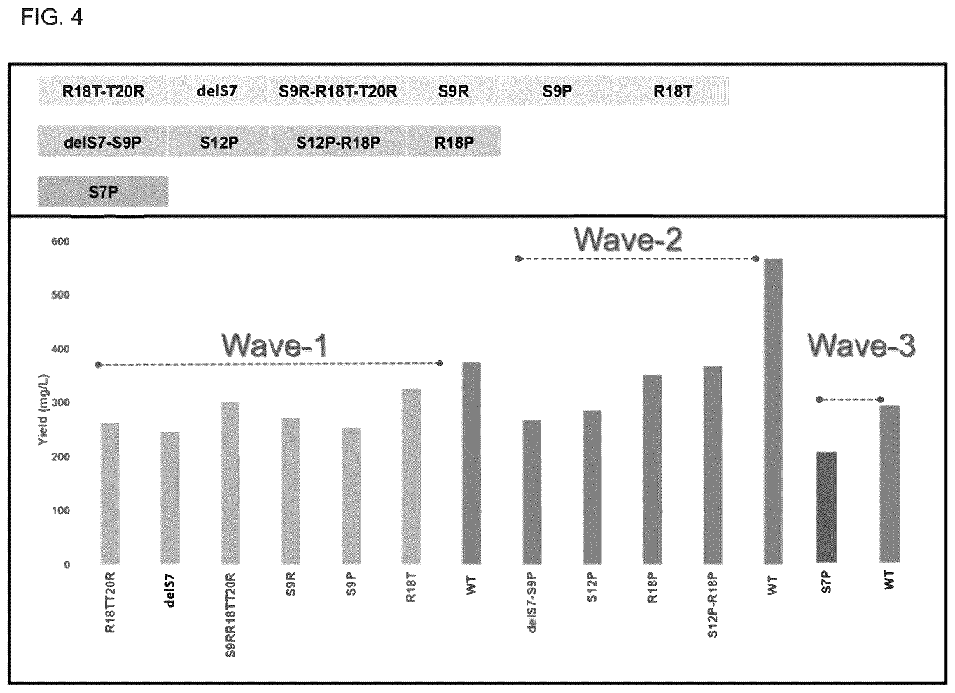

Adalimumab wild-type and 11 Protein-L binding knock-out mutants, i.e., R18T-T20R, delS7, S9R-R18T-T20R, S9R, S9P, R18T, del7-S9P, S12P, S12P-R18P, R18P, and S7P (see FIG. 4, top panel), were expressed in Expi293F cells and purified through mAbSelect SuRe column to evaluate protein titer. Wild type Adalimumab was used as a reference.

Plasmids encoding adalimumab heavy chain and light chains were transfected into 15-30 mL of Expi293F cells which were cultured in Expi293 Expression Medium in shake flasks when cell density reaches at 2.8×10⁶/mL using ExpiFectamine™ 293 Transfection Kit (Thermo fisher A14635) following manufactory protocol. ExpiFectamine™ 293 Transfection Enhancer 1 and 2 were added to the culture media in the second day. Transfected cells were continuous cultured in suspension at 37° C., with 8% CO₂, with shaking speed of 125 rpm for 7 days.

At day 7, transfected cells were removed from culture media by centrifugation at 200g for 10 min. Then the cell culture supernatants were further centrifuged at 3500g for 15 min to clear the cell debris. Finally, cell culture supernatant was filtered through 0.22 uM filter.

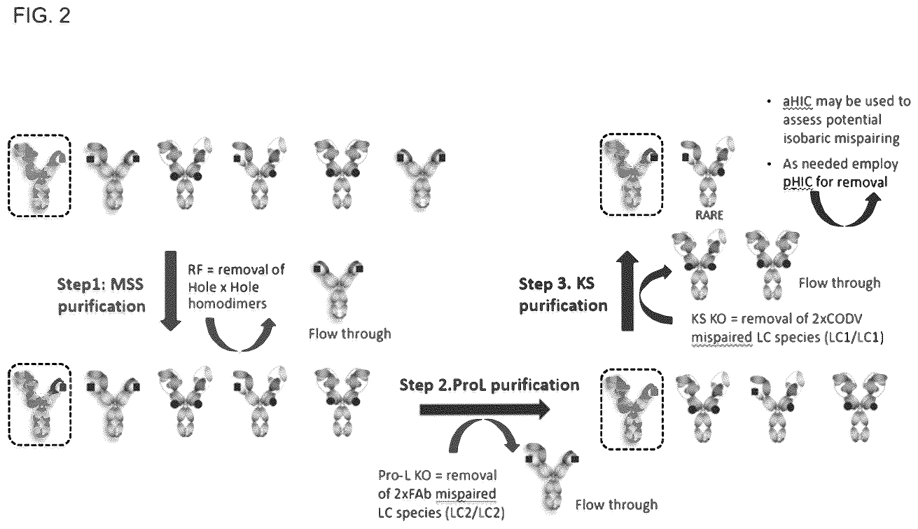

Load filtered cell culture media to 5 mL mAbSelect SuRe column (Cytiva #11-0034-95). The purification procedure is the same as that was described in Step 1 of FIG. 2.

Protein concentration after mAbSelect SuRe purification was determined by A280 nm using Nanodrop. Protein yield was calculated using the protein amount (protein concentration λ volume) divided by cell culture volume. The yield of Protein-L knock-out mutants is slightly lower than that of wild-type adalimumab (FIG. 4, bottom panel).

Biolayer interferometry (BLI) was used to assess binding of adalimumab VL FW1 variants to 100 nM pro-L ligand (ThermoFisher Inc.) in solution. Adalimumab wild-type, 10 Protein-L knock-out mutants, and a lambda light chain negative control antibody were diluted to 100 nM in binding buffer (phosphate buffered saline, pH 7.4, with 1 mg/mL bovine serum albumin), then loaded onto Anti-human IgG Fc Capture (AHC) biosensor (Sartorius, 18-5060) to density of 1.0 nm using biolayer interference system (Octet RED96e, ForteBio). After equilibrium, binding to 100 nM recombinant Protein L (Pierce™ 2118) was performed using 180 seconds association and 180 seconds dissociation at 25° C. Binding responses were captured, and binding affinity was fitted using 1:1 binding model. S12P mutation greatly decreased the binding to Pro-L while S12P_R18P double mutant completely abolished Pro-L binding.

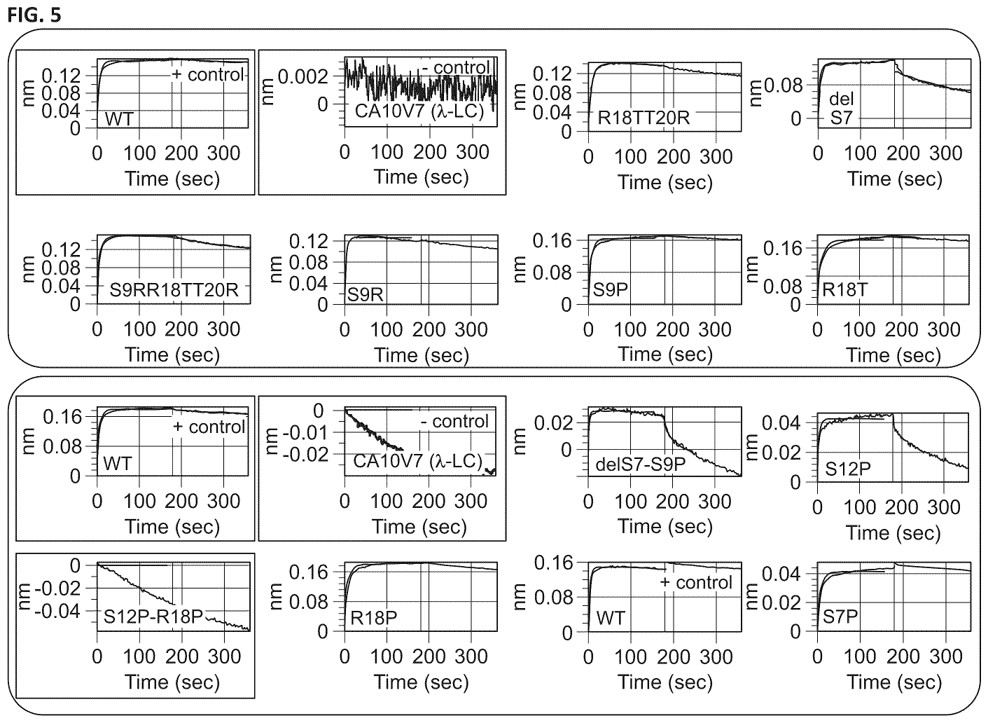

Shown in FIG. 5 are the binding curves for each adalimumab variant in wave-1 (top, which shows the binding for variants R18T-T20R, delS7, S9R-R18T-T20R, S9R, S9P, and R18T), wave-2 (bottom, which shows the binding for variants del7-S9P, S12P, S12P-R18P, and R18P), and wave-3 (bottom, which shows the binding for variant S7P), wild type Adalimumab, and an irrelevant (non-TNFα binding) negative control antibody harboring a lambda light chain (non-protein-L binding).

BLI binding of Adalimumab VL FW1 variants to 100 nM TNFα (from vendor X) in solution was assessed. Adalimumab wild-type, 10 Protein-L knock-out mutants, and a lambda light chain negative control antibody were diluted to 100 nM in Octet binding buffer (phosphate buffered saline, pH 7.4, with 1 mg/mL bovine serum albumin), then loaded onto Anti-human IgG Fc Capture (AHC) biosensor (Sartorius, 18-5060) to density of 1.0 nm using biolayer interference system (Octet RED96e, ForteBio). After equilibrium, binding to 100 nM recombinant TNFα antigen (R&D Systems, 10291-TA-050) was performed using 180 seconds association and 180 seconds dissociation at 25° C. Binding responses were captured, and binding affinity was fitted using 1:1 binding model. These Protein-L knock-out mutants remain the same antigen binding as wild-type adalimumab.

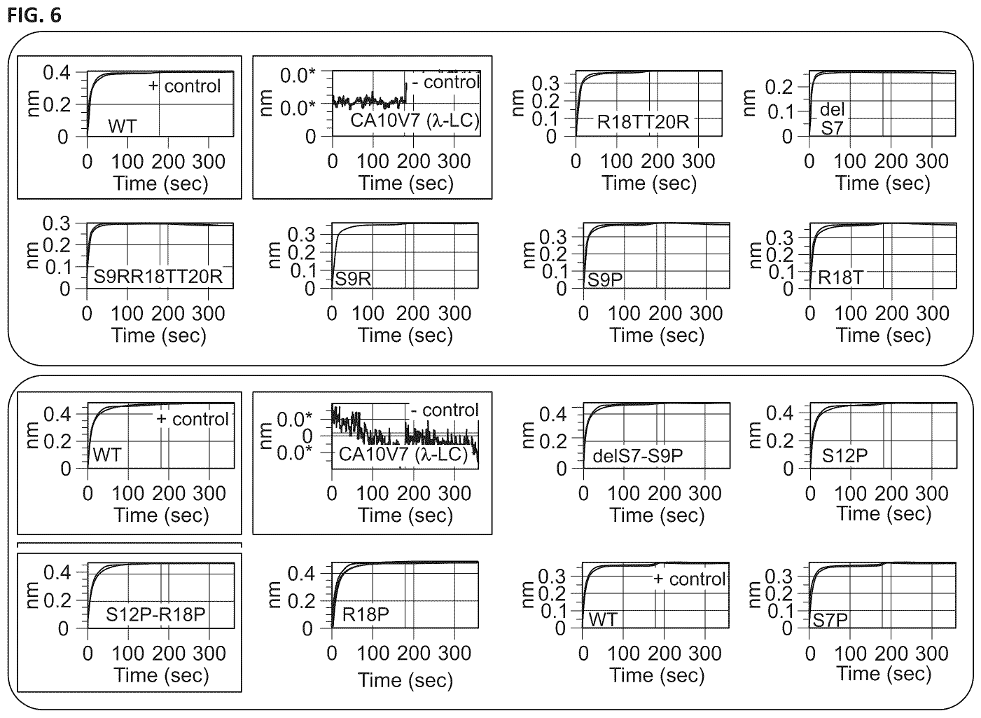

Shown in FIG. 6 are the binding curves for each Adalimumab variant in wave-1 variant in wave-i (top, which shows the binding for variants R18T-T20R, delS7, S9R-R18T-T20R, S9R, S9P, and R18T), wave-2 (bottom, which shows the binding for variants del7-S9P, S12P, S12P-R18P, and R18P), and wave-3 (bottom, which shows the binding for variant S7P), wild type Adalimumab, and an irrelevant (non-TNFα binding) negative control antibody.

Adalimumab VL FW1 variants versus wild-type binding to protein-L Resin (ThermoFisher Inc.) was evaluated. One mg of mAbSelect SuRe resin purified adalimumab wild-type and four Protein-L knock-out mutants in neutral pH buffer, were load to 1 mL of Protein L column (Pierce™, 89928) at a flow rate of 1 mL/min. Then washed with 10 column volume (CV) of phosphate buffered saline. The protein in flow-through and wash buffer were collected and combined. Followed by wash, the column was eluted with 5 CV of 50 mM sodium acetate, pH 3.5. Eluted protein was neutralized using 1M, pH 9.0 Tris buffer. The column was further eluted with 5 CV of 50 mM Glycine-HCl, pH 2.5 and elution was neutralized with Tris buffer. The protein amount in flow through and two elutions was measured using A280 nm on a Nanodrop. delS7-S9P and S12P mutants are partially flow-through (non-binding) from Pro-L column while S12P_R18P completely lost binding to this column.

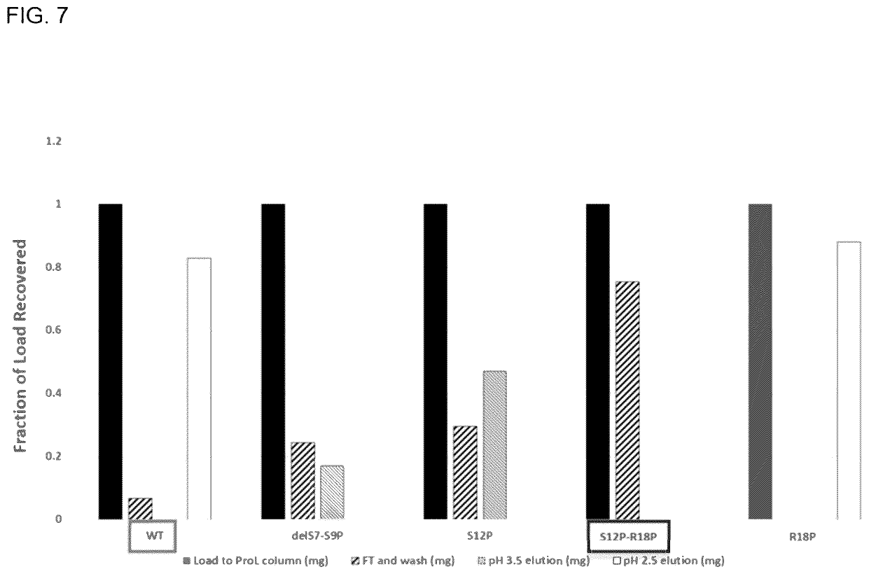

As shown in FIG. 7, load amounts were set to 1.0 and the relative levels of antibody collected in the other samples are shown reflected as fractional amounts of this.

### Example 2: Development of KappaSelect (KS) KO Mutations

KappaSelect (KS) is an affinity chromatography material constructed through the immobilization of a camelid derived heavy chain only antibody (VHH) ligand. KS resin binds selectively and with high capacity and affinity only to the constant region (CL) of kappa light chains.

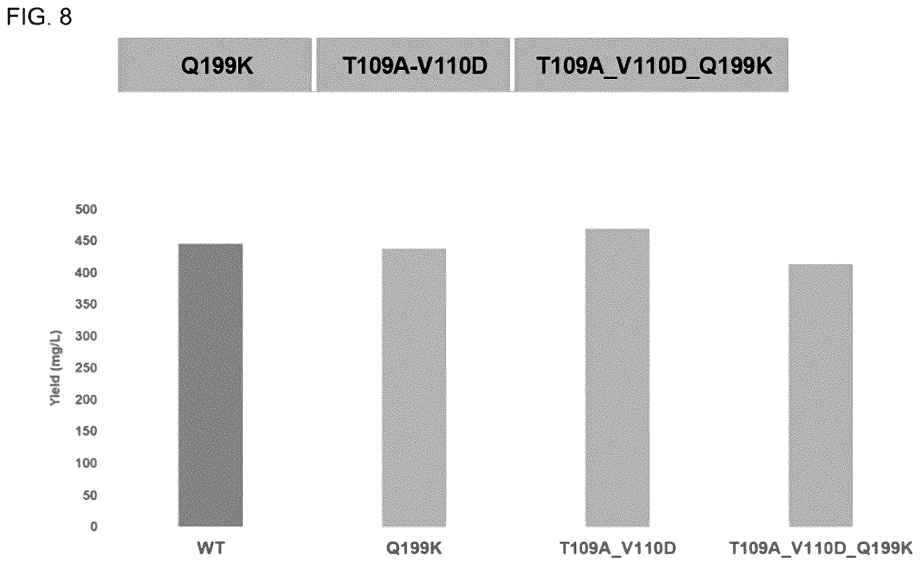

To further extend the development of the novel purification approaches mutations that would diminish or completely abrogate the binding of an antibody LC to the KS ligand and to KS affinity resin were identified. A series of single, double, or triple amino acid substitutions in the CL domain of Adalimumab were designed and tested (FIG. 8, top panel). Adalimumab variants containing these different mutant LCs were produced and characterized relative to the wild type version for KS ligand binding by BLI or KS immobilized on resin, as well as several other qualities including expression titer, interaction with the target protein TNFα, accelerated stability, and predicted immunogenicity.

Wild-type and three KappaSelect binding knock-out mutants were expressed in Expi293F cells and purified by mAbSelect SuRe affinity resin using the methods as described in FIG. 4. Three KappaSelect binding knock-out mutants has similar protein titer comparing to that of wild-type adalimumab (FIG. 8, bottom panel). Wild type Adalimumab was used as a reference.

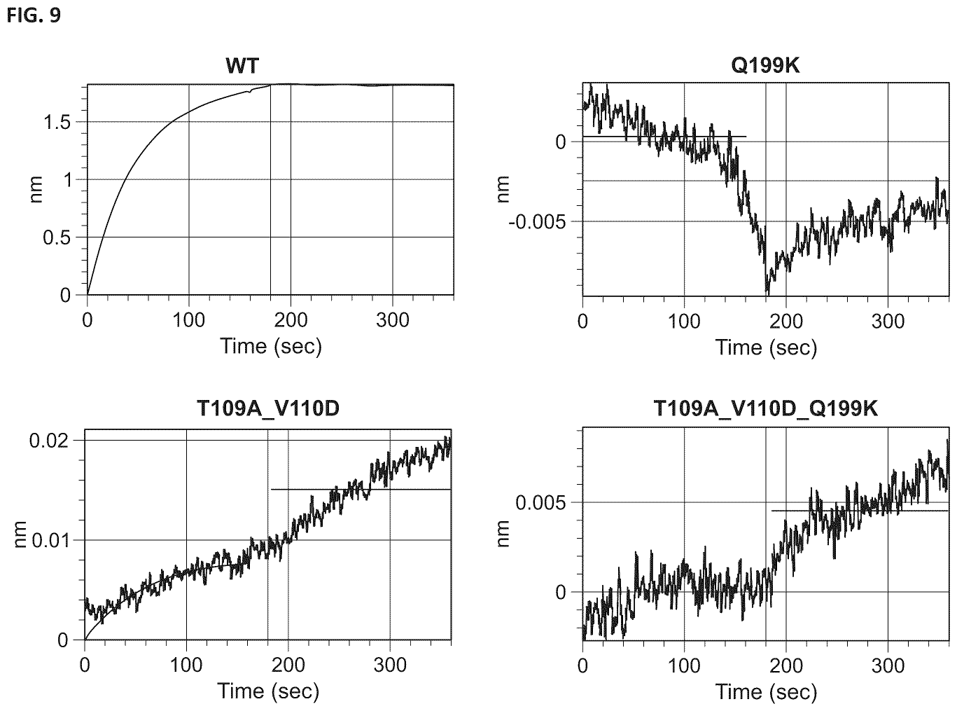

BLI binding of Adalimumab CL variants to KS ligand (Cytiva) was assessed (FIG. 9). CaptureSelect™ biotin anti-LC-kappa (human) conjugate (Thermofisher Scientific, 7103292100) was diluted in Octet binding buffer (phosphate buffered saline, pH 7.4, with 1 mg/mL bovine serum albumin) and load onto Streptavidin (SA) Biosensor (Sartorius, 18-5019), then binding to 100 nM wild-type adalimumab and three KappaSelect binding knock-out mutants in Octet binding buffer on Biolayer Interferometry (Octet RED96e, ForteBio). Binding traces and responses were captured. FIG. 9 shows binding curves for each Adalimumab variant and wild type Adalimumab. All three KappaSelect binding knock-out mutants lost binding to KappaSelect ligand.

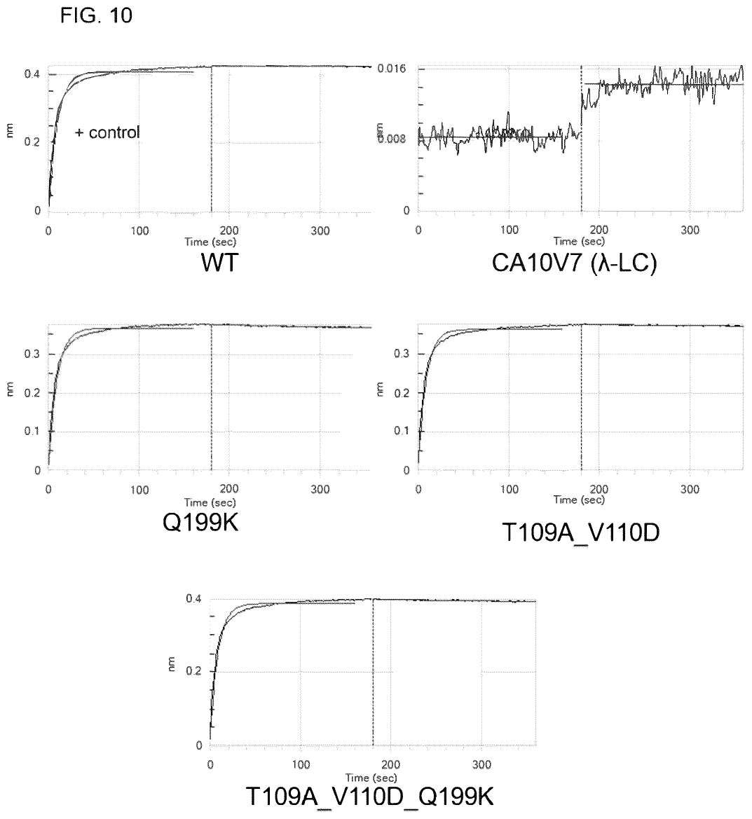

BLI binding assessment of Adalimumab CL variants to 100 nM TNFα (R&D Systems, 10291-TA-050) in solution is shown in FIG. 10. Data were generated using a Forte Bio Octet Biosensor and Anti-human IgG Fc Capture (AHC) biosensor tips (Sartorius, 18-5060) as described above for FIG. 6. Shown are the binding curves for each Adalimumab CL variant, wild type Adalimumab, and an irrelevant (non-TNFα binding) negative control antibody.

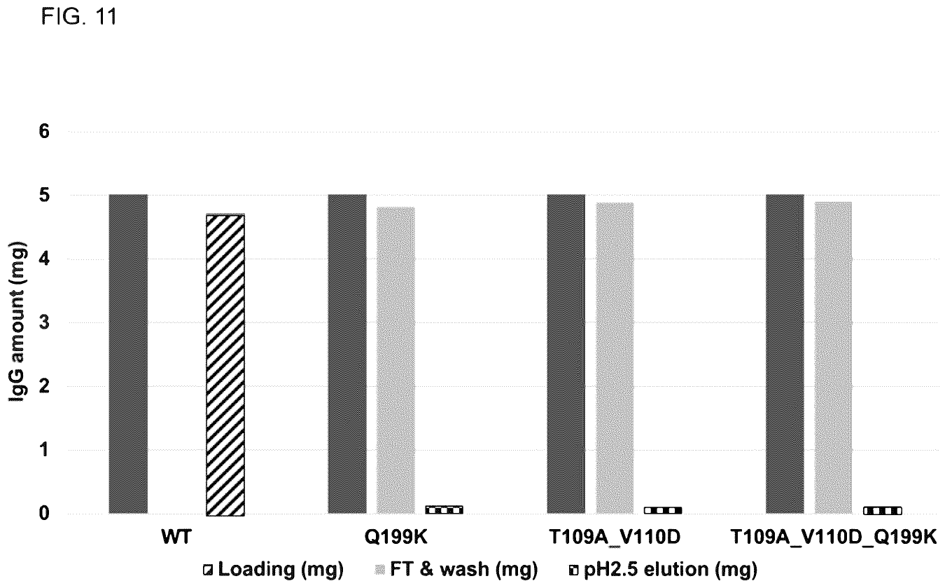

Evaluation of Adalimumab CL variants versus wild-type binding to KS Resin (Cytiva) is shown in FIG. 11. Five mg of mAbSelect SuRe resin purified adalimumab wild-type and three KappaSelect knock-out mutants in neutral pH buffer, were load to 1 mL of HiTrap KappaSelect columns (Cytiva 17545811) at a flow rate of 1 mL/min. Columns were then washed with 10 column volume (CV) of phosphate buffered saline. The protein in flow-through and wash buffer were collected and combined. Followed by wash, the column was eluted with 5 CV of 50 mM Glycine-HCl, pH 2.5 elution buffer and elution was neutralized with 1M, pH 9.0 Tris buffer. The protein amount in flow through and elution was measured using A280 nm on a Nanodrop. All three KappaSelect knock-out mutants are in flow-through with no binding to KappaSelect column. In the graphs shown in FIG. 11, initial load amounts in mg are shown for comparison to the quantities of antibody collected in the other fractions.

### Example 3: Proof of Principle Study for Affinity-Based Removal of Mispaired LC Multispecific Antibody Species Using Non-Pro-L Binding LC

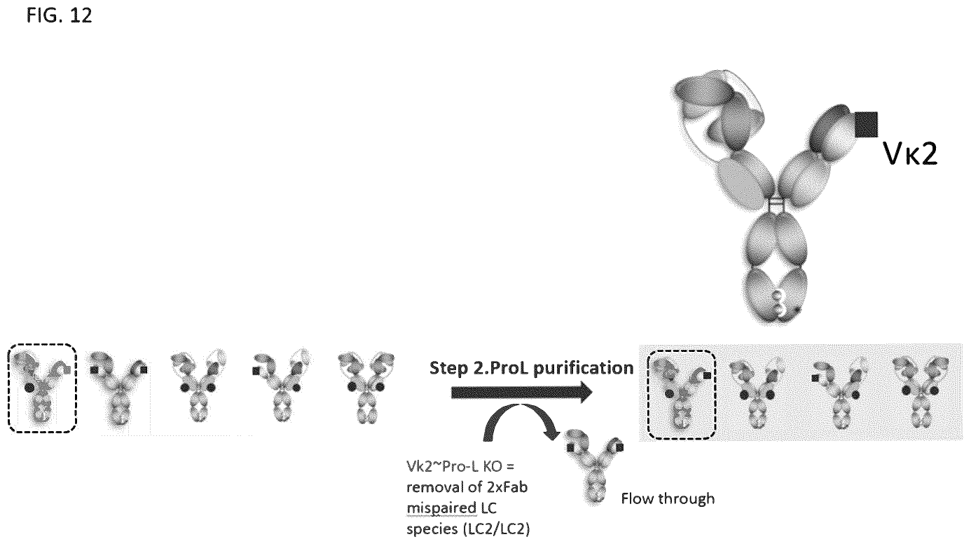

A trispecific CODV containing antibody, harboring a Vk2 Fab arm LC non-competent for pro-L binding and a non-Vk2light chain which is capable for pro-L binding. CODV arm LC capable of pro-L binding, was expressed then processed over MSS (Cytiva) (FIG. 12). The MSS eluted material containing the triAb of interest as well as 2×FabLC and 2×CODV LC mispaired species was then further purified in a second step over protein-L resin.

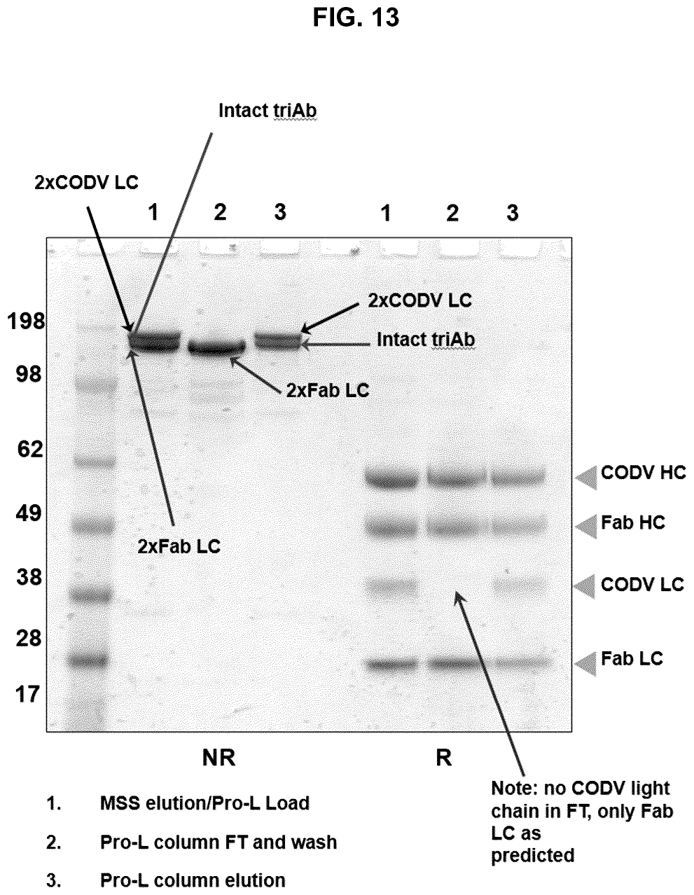

FIG. 13 shows a Coomassie-stained SDS-PAGE gel of samples from the trispecific CODV 2-step purification. Shown are the MSS elution/pro-L load, Pro-L FT/wash, and Pro-L elution under both reducing and non-reducing conditions. The second step purification over pro-L resulted, as expected, in the selective removal of the 2×FabLC mispaired species due to the presence of the Vk2 Fab arm LC non-competent for interaction with pro-L.

### Example 4: Additional Characterizations of Adalimumab Variants with Pro-L KO Mutations

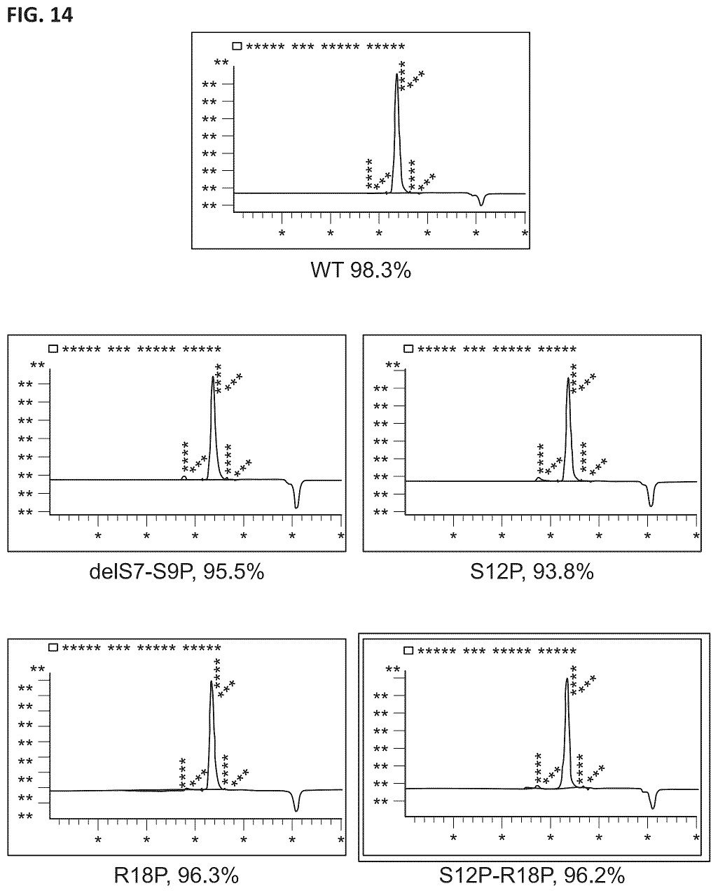

Analytical size exclusion chromatography (aSEC) data of wild type and VL FW1 mutant versions of Adalimumab post-MSS purification is shown in FIG. 14. Percent of expected main peak is shown.

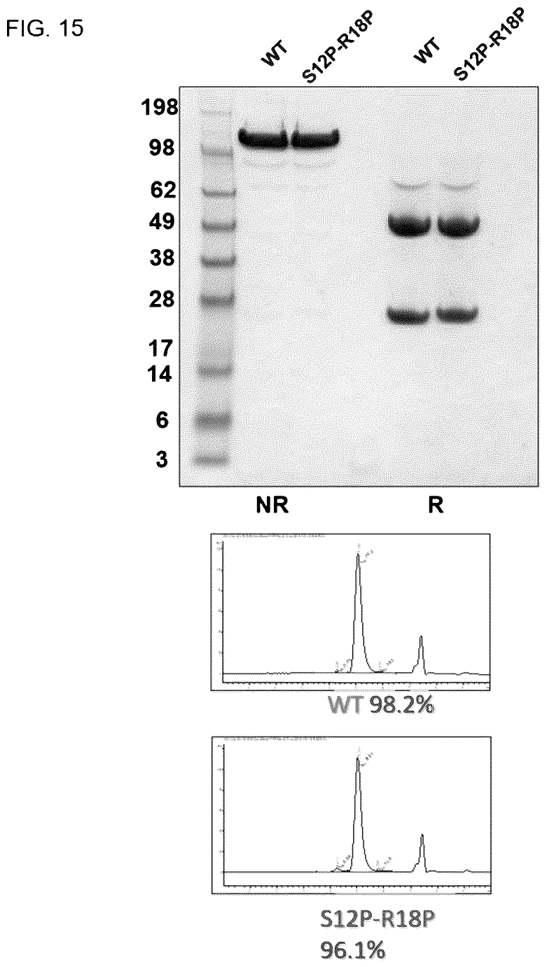

FIG. 15 shows the analysis of samples of wild type adalimumab and the S12P-R18P mutant taken at the end of an accelerated stability test (50 mM HEPES, pH 7.4, 37° C. for 2 weeks). Top panel shows a Coomassie-stained SDS-PAGE gel (NuPAGE™ 4 to 12%, Bis-Tris, Invitrogen, NP0321BOX) of non-reduced and reduced samples is shown. Bottom panels show aSEC data for each respective sample. Percent of expected main peak is shown.

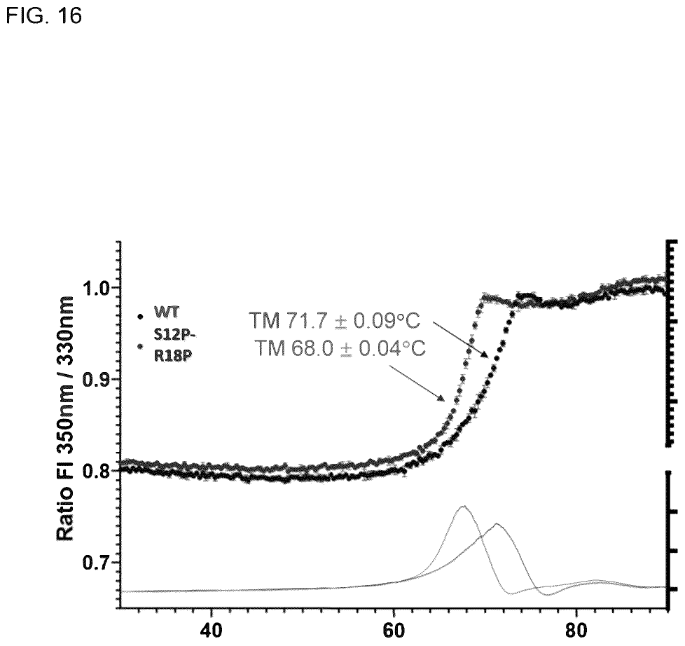

Adalimumab wild-type and Pro-L KO S12P-R18P mut were buffered in 10 mM pH 6.0 histidine buffer, and protein concentrations were normalized to 0.5 mg/mL. Differential Scanning Fluorimetry (nano-DSF) data and derived Tm from NanoTemper Prometheus are shown in FIG. 16. Nine-μd samples were loaded into the respective capillaries (NanoTemper PR-C006) and the temperature was equilibrated at 20° C. for 3 min before ramping up to 95° C. at 1° C./min. The fluorescence emission (330 nm and 350 nm) was recorded as a function of temperature. The maxima of the first derivative of the ratio of fluorescence intensities at 330 nm and 350 nm as a function of temperature were used to determine the melting temperature of the proteins.

### Example 5A. Additional Characterizations of Adalimumab Variants with KS KO Mutations

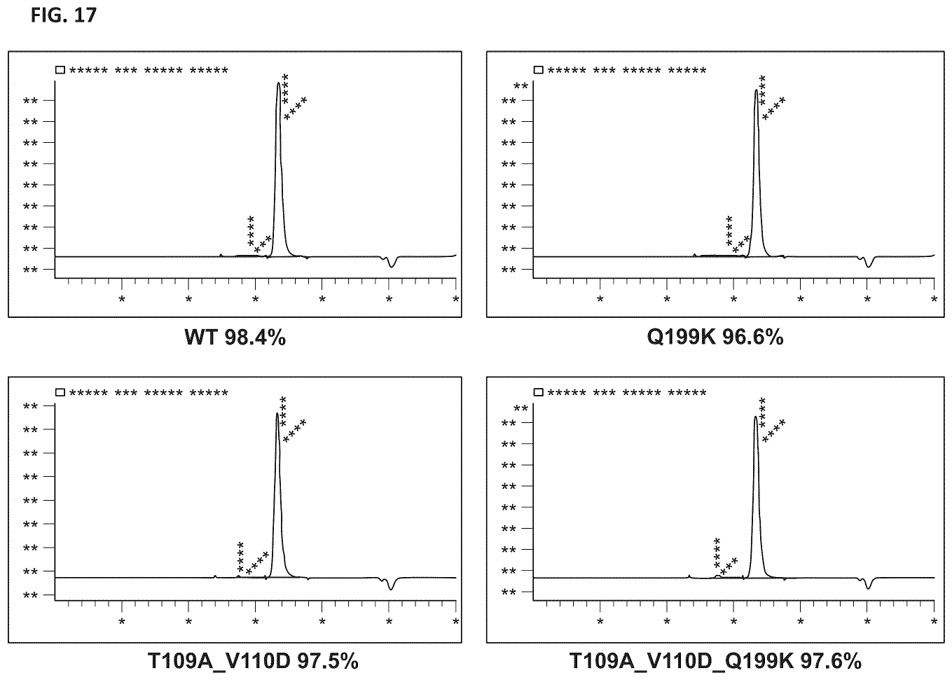

Analytical size exclusion chromatography (aSEC) data of wild type and CL mutant versions of Adalimumab post-MSS purification were performed as described above. Results are shown in FIG. 17. Percent of expected main peak is shown.

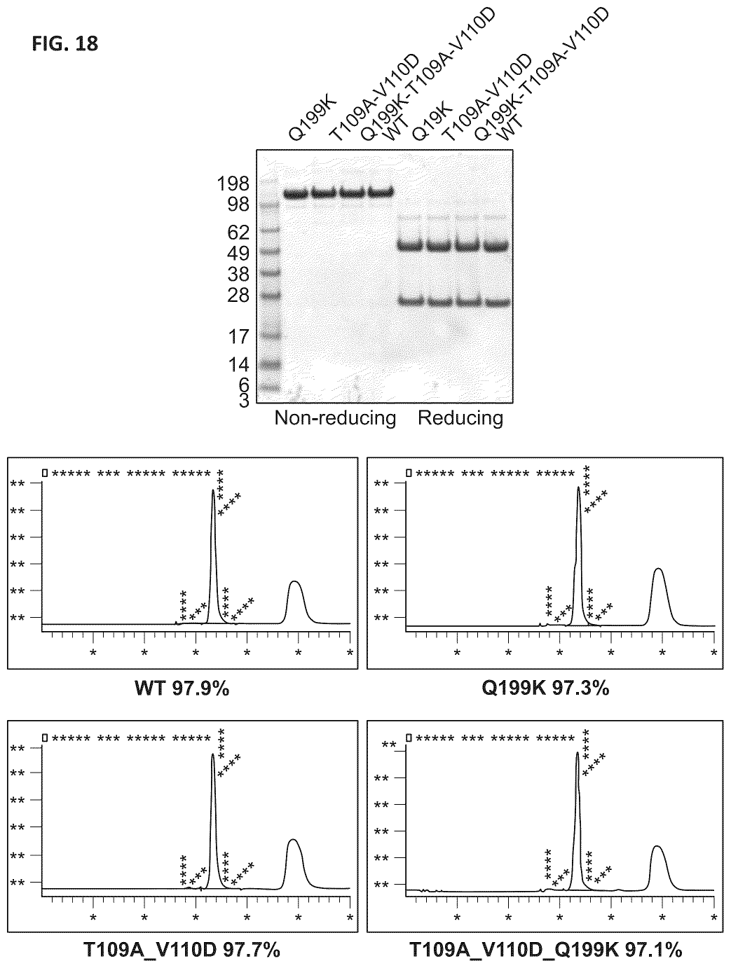

Analysis of samples of WT Adalimumab and the three CL mutants taken at the end of an accelerated stability test (40° C. for 2 weeks) is shown in FIG. 18. Top panel shows a Coomassie-stained SDS-PAGE gel of non-reduced and reduced samples is shown. Bottom panels show aSEC data for each respective sample. Percent of expected main peak is shown.

DSC analysis of WT and KappaSelect KO mutants of adalimumab at F(ab)′2 level was performed.

1 mg of wild-type and three KappaSelect KO mutants of adalimumab was digested with IdeZ protease (New England Biolabs, P0770S) at 37° C. overnight. The next day, 5 μg of digested antibodies were analyzed on SDS-PAGE to confirm F(ab)′2 were completely cleaved from Fc. The rest of protease digested antibody samples were loaded on 1 mL of CaptureSelect CH1-XL affinity resin (Thermo Scientific 494346201). After washing with PBS, samples were eluted with 5 column volume of 0.1 M glycine buffer, pH 2.5, and neutralized with 1M Tris, pH 9.0.

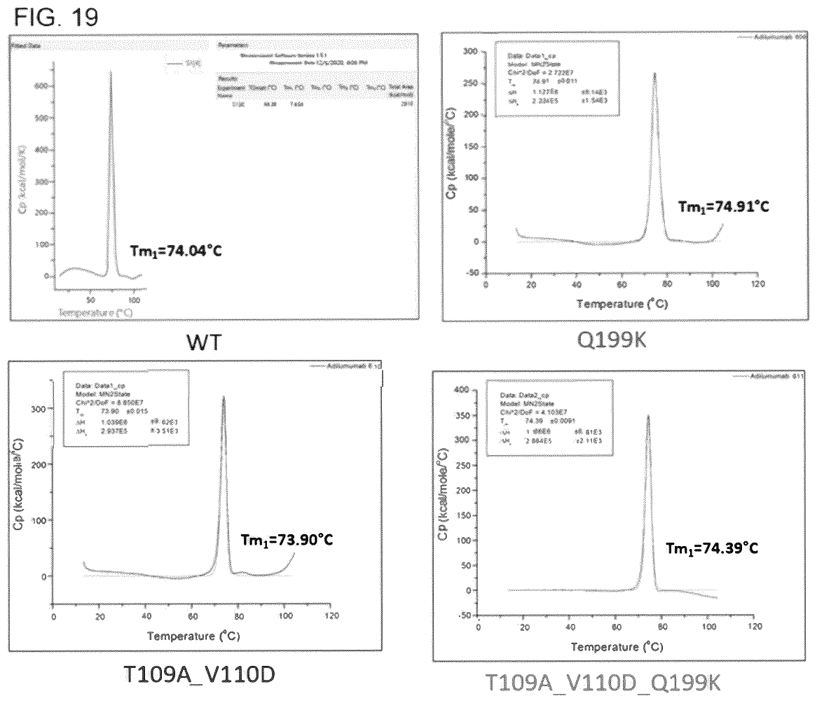

Samples were buffer exchanged in 10 mM histidine pH 6.0, and Differential Scanning Calorimetry (DSC) was performed on a MicroCal PEAQ-DSC system (Malvern Panalytical) from 15-105° C., at 200° C./h scan rate. Results are shown in FIG. 19. After background subtraction with buffer only reference, the protein's heat capacity (Cp) as a function of temperature was shown. Tm (melting point) was calculated from the melting curve.

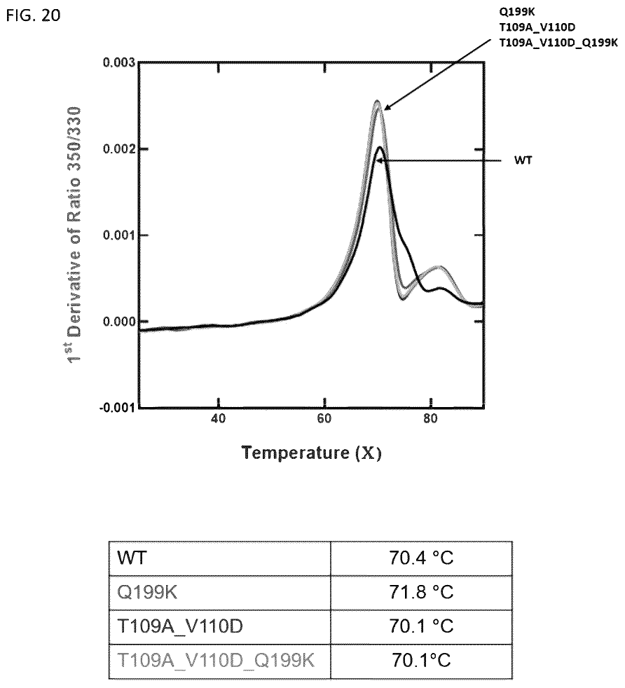

Differential Scanning Fluorimetry (nano-DSF) data and derived Tm are shown for wild-type adalimumab and the three CL mutants for knocking out KappaSelect resin binding in FIG. 20. The DSF method used is the same as that was described in FIG. 16.

### Example 5B. Proof of Principle Study for Affinity-Based Removal of Mispaired LC Multispecific Antibody Species Using Non-KS Binding LC

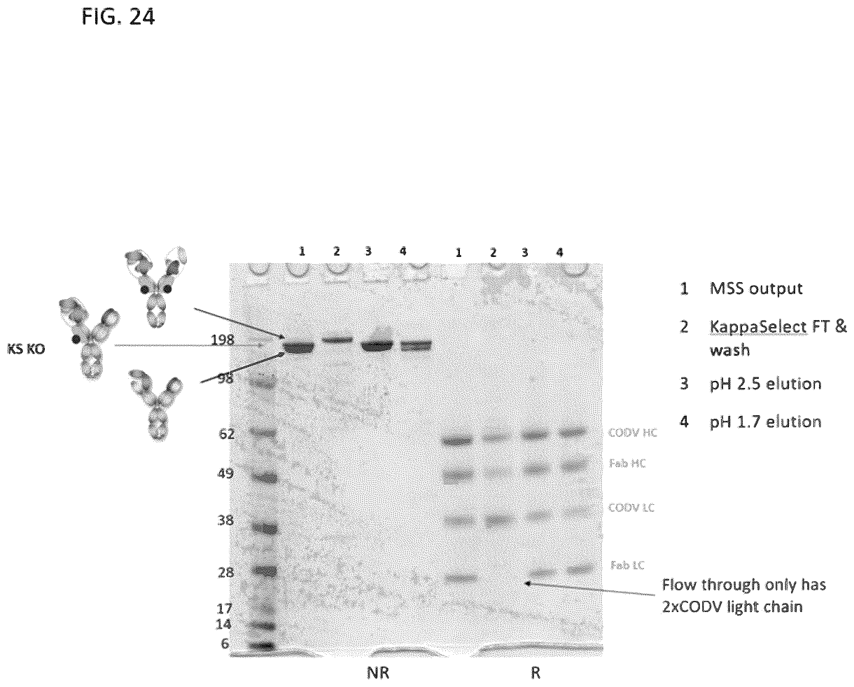

A trispecific CODV with a single Q199K mutation in its CODV light chain was expressed using the Expi293 system and purified using Protein A followed by KappaSelect chromatography. SDS-PAGE demonstrated the presence of 2× CODV LC trispecific Ab, 2× Fab LC trispecific Ab, and the desired intact trispecific Ab in the Protein A eluate (FIG. 24). The KappaSelect flow through and two low pH elution fractions (pH 1.7 and 2.5) were collected and analyzed by SDS-PAGE. The KappaSelect flow through mainly contained the 2× CODV LC trispecific Ab as expected while the eluate did not have this mis-paired contaminant (FIG. 24), indicating that the KappaSelect knock-out mutation on the CODV arm enabled removal of the 2× CODV LC tsAb species.

### Example 6: A Three-Step Affinity Purification Procedure for CODV Antibodies

A typical three-step affinity purification procedure consisting of mAbSelect SuRe, KappaSelect and Protein-L for multi-specific antibodies was evaluated.

In a first purification evaluation a CODV trispecific antibody was purified using a three step affinity purification process. A Q199K KS KO mutation was introduced into the light chain of the CODV arm and S12P-R18P ProL KO mutations were introduced to the light chain of the Fab arm. The antibody also had knob mutations in the CODV heavy chain and hole mutations in the heavy chain of the Fab arm.

In a second purification evaluation a CODV trispecific antibody was purified using a three step affinity purification process. A Q199K KS KO mutation was introduced into the light chain of the Fab arm and S12P-R18P ProL KO mutations were introduced to the light chain of the CODV arm. The antibody also had knob mutations in the CODV heavy chain and hole mutations in the heavy chain of the Fab arm.

The cell culture supernatant was harvested after protein expression in Expi293F cells or ExpiCHO-S cell after 4-14 days. Then filter on 0.2 μm PES filter unit (or equivalent) to remove any particulates.

Step 1: mAbSelect SuRe affinity purification was performed using the following conditions.
Column: mAbSelect SuRe (Cytiva #11-0034-95)
Dimensions: 5 mL column (16 mm×25 mm)
Default flow rate: 5 mL/min (max ˜20 mL/min)

Buffers:
A: 0.2 M NaOH
B: 20 mM sodium phosphate, 150 mM NaCl, pH 7.2
C: 50 mM sodium acetate, pH 3.5
D: 50 mM succinic acid
E: 20% ethanol

Load volume: The amount of mAbSelect SuRe resin needed was calculating using 30 mg IgG/mL resin. A 5 mL column will capture>150 mg IgG in cultured cell supernatant.

Column operation:
Sanitize 4 CV Buffer A for 15 minutes contact time
Rinse with 5 CV MilliQ water
Equilibrate with 15 CV Buffer B
Loaded cell culture supernatant containing antibody
Washed with 10 CV Buffer B
Eluted with 5 CV Buffer C in tube containing 1/10 volume (2.5 mL) 1M Tris pH 9 to neutralize
Stripped column with 5 CV buffer D collect in container with 600 mM Sodium phosphate dibasic to neutralize. This is just in case Ab elutes late as sometimes seen with IgG₂.
Cleaned column with 10 CV Buffer A
Rinsed with 5 CV MilliQ water
Re-equilibrated with 10 CV Buffer B
Washed with 10 CV Buffer E and store at 4° C.

mAbSelect SuRe elution containing antibodies in neutral pH buffer was ready for next step of affinity purification. Protein concentration was measured using A280 nm with a Nanodrop. The protein amount used for next step KappaSelect affinity purification was calculated so that it did not over the binding capacity.

Step 2: KappaSelect affinity purification was performed using the following conditions.
Column: HiTrap KappaSelect 5×1 mL (Cytiva 17545811)
Dimensions: 1 mL pre-pack column
Default flow rate: 1 mL/min

Buffers:
F: 10 mM NaOH, pH 12
G: 0.1 M glycine buffer, pH 2.5
H: 0.1 M citric acid, pH 2.1
Sample input: neutralized protein elution MSS.
Load volume: how much KappaSelect resin needed was calculated using 15 mg IgG/mL resin.
Column operation:
1) Clean-In-Place 10CV Buffer F for 15 minutes contact time. Leaving the column in this high pH buffer for excess time will damage the column.
2) Rinsed with 5 CV MilliQ water
3) Equilibrated with 10 CV Buffer B
Loaded neutralized protein elution from mAbSelect SuRe step 1.6 to KappaSelect column(s)
5) Washed with 10 CV Buffer B
6) Eluted with 5 CV Buffer Gin tube containing 1/10 volume 1M Tris pH 9 to neutralize to pH 7-8
7) Stripped column with 5 CV buffer H. Leave the column in this low pH buffer for excess time will damage the column.
8) Cleaned column with 10 CV Buffer F. Leave the column in this high pH buffer for excess time will damage the column
9) Rinsed with 5 CV MilliQ water
10) Re-equilibrated with 10 CV Buffer B
11) Washed with 10 CV Buffer E and store at 4° C.

KappaSelect affinity elution containing antibodies in neutral pH buffer was ready for next step of Protein-L affinity purification. Protein concentration was measured using A280 nm with a Nanodrop. The amount of protein used for next step affinity purification was calculated so that it did not exceed the binding capacity.

Step 3: Protein-L affinity purification was performed using the following conditions.
Column: Thermo Scientific 89929 Pierce Chromatography Cartridges Protein L, 1× 5 mL
Dimensions: 1 mL pre-pack column
Default flow rate: 1 mL/min
Sample input: neutralized protein elution from KS resin
Load volume: how much Protein-L resin needed using 4-5 mg human IgG/mL of resin bed was calculated.

Column operation:
1) Clean-In-Place 10 CV Buffer F for 15 minutes contact time. Leaving the column in this high pH buffer for excess time will damage the column.
2) Rinsed with 5 CV MilliQ water
3) Equilibrated with 10 CV Buffer B
4) Loaded neutralized protein elution from KappaSelect step to Protein-L column(s)
5) Washed with 10 CV Buffer B
6) Eluted with 5 CV Buffer G in tube containing 1/10 volume 1M Tris pH 9 to neutralize to pH 7-8
7) Cleaned column with 10 CV Buffer F. Leave the column in this high pH buffer for excess time will damage the column
8) Rinsed with 5 CV MilliQ water
9) Re-equilibrated with 10 CV Buffer B
10) Washed with 10 CV Buffer E and stored at 4° C.

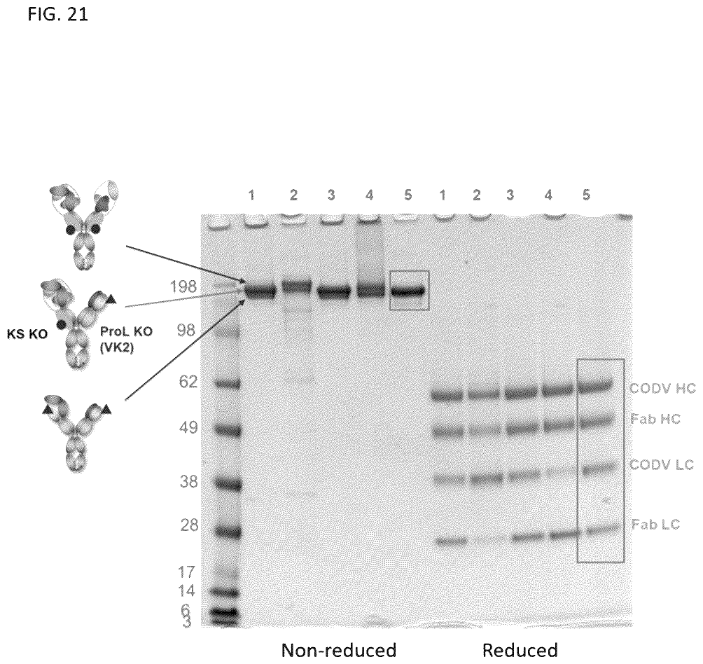

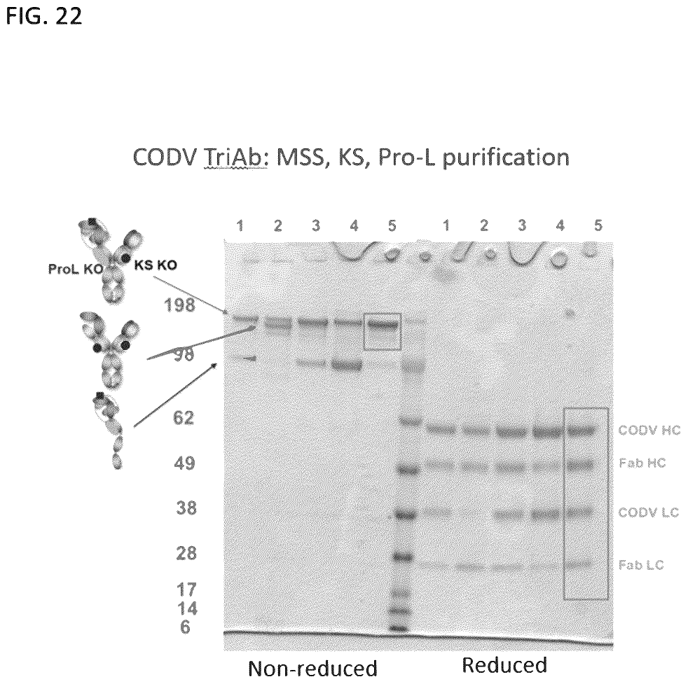

Results for the first evaluation (KS KO on CODV arm/ProL KO on Fab arm) are shown in FIG. 21 and results for the second evaluation (KS KO on Fab arm/ProL KO on CODV arm) are shown in FIG. 22.

### Example 7. A Three-Step Affinity Purification Procedure for Bispecific Antibodies

A typical three-step affinity purification procedure as described in Example 6 was evaluated. The bispecifc antibody contained a trastuzumab arm and pertuzumab arm. The trastuzumab arm contained S12P-R18P ProL KO mutations and knob mutations and the pertuzumab arm contained Q199K KS KO mutation and hole mutations. The pertuzumab arm also contained RF mutations in the CH3 domain.

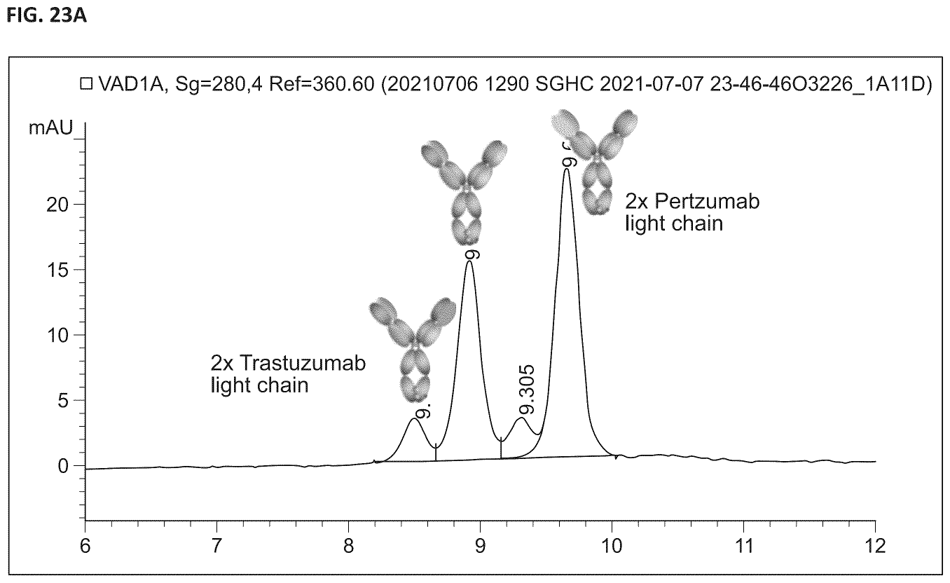

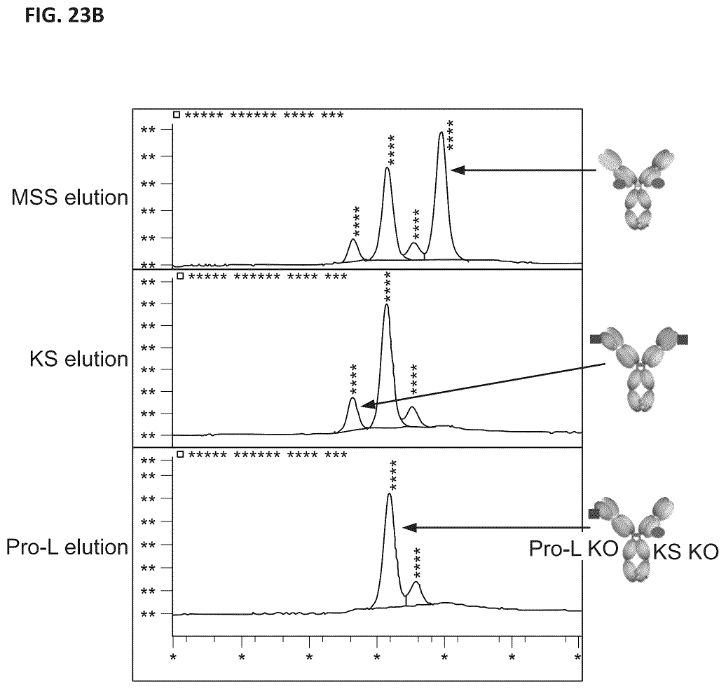

Results are shown of the MabSelect Sure elution are shown in FIG. 23A. Results of the MabSelect Sure elution, KS elution and Pro-L elution are shown in FIG. 23B.

### Example 8: A Two-Step Affinity Purification Procedure for Multivalent Antibodies

A typical two-step affinity purification procedure for a multivalent antibody consisting of KappaSelect and Protein-L for multi-specific antibodies is evaluated. The multivalent antibody comprises two light chains and two heavy chains.

A Q199K KS KO mutation is introduced into one light chain of the multivalent antibody and S12P-R18P ProL KO mutations are introduced to the other light chain of the multivalent antibody.

The multivalent antibody is produced in host cells. The cell culture supernatant is harvested after protein expression after 4-14 days. The cell culture supernatant is then filtered on 0.2 μm PES filter unit (or equivalent) to remove any particulates.

Step 1: KappaSelect affinity purification is performed using the following conditions.
Column: HiTrap KappaSelect 5×1 mL (Cytiva 17545811)
Dimensions: 1 mL pre-pack column
Default flow rate: 1 mL/min
Buffers:
F: 10 mM NaOH, pH 12
G: 0.1 M glycine buffer, pH 2.5
H: 0.1 M citric acid, pH 2.1
Sample input: neutralized protein elution MSS.
Load volume: how much KappaSelect resin needed is calculated using 15 mg IgG/mL resin.

Column operation:
1) Clean-In-Place 1OCV Buffer F for 15 minutes contact time. Leaving the column in this high pH buffer for excess time will damage the column.
2) Rinse with 5 CV MilliQ water
3) Equilibrate with 10 CV Buffer B
Load fileted cell culture supernatant to KappaSelect column
5) Wash with 10 CV Buffer B
6) Elute with 5 CV Buffer G in tube containing 1/10 volume 1M Tris pH 9 to neutralize to pH 7-8. Collect fractions comprising multivalent antibody.
7) Strip column with 5 CV buffer H.
8) Clean column with 10 CV Buffer F.
9) Rinse with 5 CV MilliQ water
10) Re-equilibrate with 10 CV Buffer B
11) Wash with 10 CV Buffer E and store at 4° C.

KappaSelect affinity elution containing antibodies in neutral pH buffer is ready for next step of Protein-L affinity purification. Protein concentration is measured using A280 nm with a Nanodrop. The amount of protein used for next step affinity purification is calculated so that it does not exceed the binding capacity.

Step 2: Protein-L affinity purification is performed using the following conditions.
Column: Thermo Scientific 89929 Pierce Chromatography Cartridges Protein L, 1×5 mL
Dimensions: 1 mL pre-pack column
Default flow rate: 1 mL/min
Sample input: neutralized protein elution from KS resin
Load volume: how much Protein-L resin needed using 4-5 mg human IgG/mL of resin bed is calculated.

Column operation:
1) Clean-In-Place 10 CV Buffer F for 15 minutes contact time. Leaving the column in this high pH buffer for excess time will damage the column.
2) Rinse with 5 CV MilliQ water
3) Equilibrate with 10 CV Buffer B
4) Load neutralized protein elution from KappaSelect step to Protein-L column(s)
5) Wash with 10 CV Buffer B
6) Elute with 5 CV Buffer G in tube containing 1/10 volume 1M Tris pH 9 to neutralize to pH 7-8. Collect factions comprising multivalent antibody.
7) Clean column with 10 CV Buffer F.
8) Rinse with 5 CV MilliQ water
9) Re-equilibrate with 10 CV Buffer B
10) Wash with 10 CV Buffer E and stored at 4° C.

### Example 9. Development of Additional KappaSelect (KS) KO Mutations

An additional series amino acid substitutions in the CL domain of an antibody light chain (LC) were designed and tested to identify substitutions, or combinations of substitutions, that diminished or completely abrogated the binding of the light chain to KS ligand and KS affinity resin (see, e.g., Example 2). Adalimumab, an anti-human TNF alpha antibody comprising kappa light chains and a human IgG Fc region variant comprising L234A and L235A mutations (wherein amino acid numbering is according to the EU index), was used as an exemplary antibody. Adalimumab variants comprising two mutant LCs that comprised (i) an H198R substitution, wherein amino acid numbering is according the EU index, (ii) a Q199W substitution, wherein amino acid numbering is according to the EU index, or (iii) T 109R and S202R substitutions, wherein amino acid numbering is according to the EU index, were expressed in HEK293T cells. The expression titer of each adalimumab variant was determined using BioLayer Interferometry via Protein A Biosensors. See Table A below. The supernatant was purified via Protein A chromatography and the concentration of the purified proteins was determined using UV-280 absorption. See Table A below.

The binding of the adalimumab variants to the Kappa Select column ligand was analyzed via BioLayer Interferometry (BLI). The ligand (TPP-13443, EFF-18-070-1, Llama Anti-human LC-kappa VHH Single Domain Antibody [Biotin] (CAT #: NABG-118) https://www.creativebiolabs.net/Anti-LC-kappa-VHH-Single-Domain-Antibody-Biotin-19155.htm) was loaded on Streptavidin-Biosensors and the affinity of the variants was measured. The results are presented in Table A below. (See column BLI (Response in %)).

TABLE A

| Light Chain | | | % Response BLI of |
|---|---|---|---|
| Mutation(s) | | Final Protein | construct in |
| (amino acid | | Amount after | comparison to |
| numbering | Expression | Protein A | wildtype |
| according to | Yield | purification | (=% Binding to |
| the EU Index) | (mg) | (mg) | Kappa-ligand) |
| His198Arg | 15 | 10.3 | 7.87 |
| Gln199Trp | 54.7 | 30.2 | 1.66 |
| Thr109Arg + | 70.0 | 35.0 | 12.47 |
| Ser202Arg | | | |
| WT light chain | no data | no data | 100.00 |
| (no mutations) | | | |

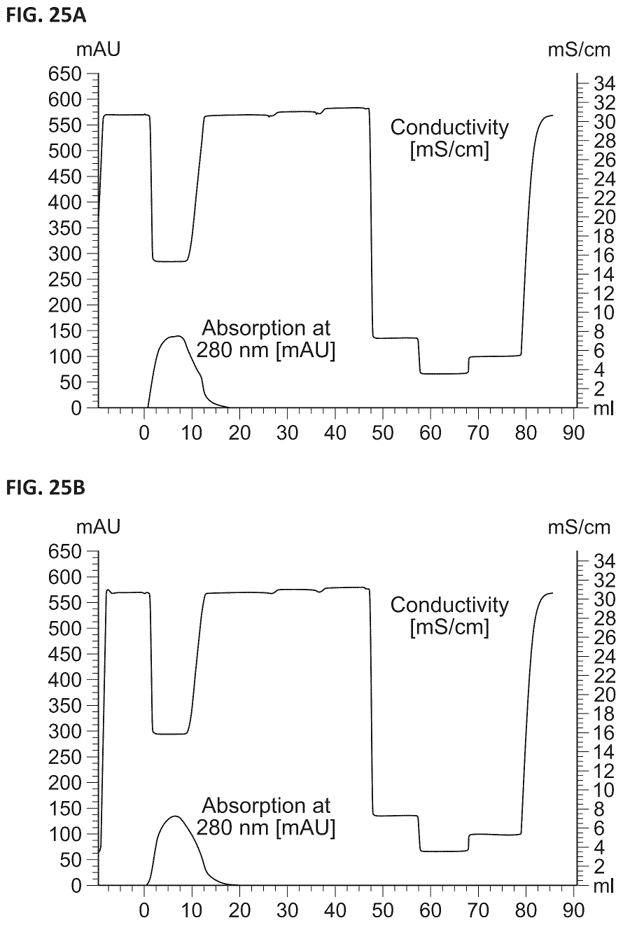

The loss of interaction of the adalimumab variants to the Kappa Select column was confirmed using a HiTrap Kappa Select column from GE Healthcare Life Science and ÄKTA Pure. A chromatogram for the adalimumab variant comprising His198Arg substituted light chains is shown in FIG. 25A and a chromatogram for the adalimumab variant comprising G199Trp substituted light chains are shown in FIG. 25B.

To confirm that the stability and specific binding to the target TNF alpha by adalimumab variants was not affected by the light chain substitutions, an analytical size exclusion chromatography (Method:TSKgel SuperSW3000) and surface plasmon resonance (Biacore 8K, Method:TOS,AG-514 (human TNF alpha Miltenyi from yeast Catalog no. 130-094) was performed. The results are shown in Tables B and C below.

TABLE B
Size Exclusion Chromatography

| Light Chain | | | | |
|---|---|---|---|---|
| Mutation(s) | | | | |
| (amino acid | | High MW | | % High MW |
| numbering | | Species- | % Purity | Species- |
| according to the | Purity | Aggregation | to | Aggregation |
| EU Index) | (%) | (%) | wildtype | to wildtype |
| His198Arg | 90.50 | 9.50 | 91.04 | 1610.17 |
| Gln199Trp | 98.41 | 1.59 | 98.99 | 269.49 |
| Thr109Arg + | 99.16 | 0.84 | 99.75 | 142.37 |
| Ser202Arg | | | | |
| WT light chain | 99.41 | 0.59 | 100.00 | 100.00 |
| (no mutations) | | | | |

TABLE C
Surface Plasmon Resonance

| Light Chain | | | | | | | |
|---|---|---|---|---|---|---|---|
| Mutation(s) (amino | | | | | | | |
| acid numbering | | | | | | | |
| according to the | | | | % KD to | % kₒₙ to | % k_{off} to | Binding |
| EU Index) | KD (nM) | kₒₙ (1/(M*s)) | k_{off} (1/s) | wildtype | wildtype | wildtype | (Qualitative) |
| His198Arg | 5.53E−11 | 3.80E+06 | 2.10E−04 | 104.14 | 92.68 | 92.68 | Yes |
| Gln199Trp | 5.28E−11 | 3.94E+06 | 2.08E−04 | 99.44 | 96.10 | 96.10 | Yes |
| Thr109Arg + | 5.83E−11 | 3.71E+06 | 2.16E−04 | 109.79 | 90.49 | 90.49 | Yes |
| Ser202Arg | | | | | | | |
| WT light chain | 5.31E−11 | 4.10E+06 | 2.17E−04 | 100.00 | 100.00 | 100.00 | Yes |
| (no mutations) | | | | | | | |

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

1: A multivalent binding protein comprising four polypeptide chains that form two antigen binding domains; wherein the four polypeptide chains comprise:
a first heavy chain polypeptide comprising a structure represented by the formula:
VH₁-CH₁ [I],
a first light chain polypeptide chain comprising a structure represented by the formula:
VL₁-CL₁ [II],
a second heavy chain polypeptide comprising a structure represented by the formula:
VH₂-CH1 [III],
and a second light chain polypeptide chain comprising a structure represented by the formula:
VL₂-CL₂ [IV];
wherein:
VL₁ is a first immunoglobulin light chain variable domain;
VL₂ is a second immunoglobulin light chain variable domain, wherein VL₂ is a κ1, κ3, or κ4 subtype light chain variable domain,
CL₁ is a first immunoglobulin light chain constant domain, wherein CL₁ is a Cκ subtype light chain constant domain;
CL₂ is a second immunoglobulin light chain constant domain;
VH₁ is a first immunoglobulin heavy chain variable domain;
VH₂ is a second immunoglobulin heavy chain variable domain;
CH₁ is an immunoglobulin heavy chain constant domain;
wherein CL₁ comprises one or more amino acid substitutions which reduce binding to a KappaSelect chromatography material compared to a CL₁ without the one or more amino acid substitutions,
wherein VL₂ comprises one or more amino acid substitutions which reduce binding to a protein L chromatography material compared to a VL₂ without the one or more amino acid substitutions, and
wherein VH₁ and VL₁ associate to form a first antigen binding domain and VH₂ and VL₂ associate to form a second antigen binding domain.

2: A multivalent binding protein comprising four polypeptide chains that form two antigen binding domains; wherein the four polypeptide chains comprise:
a first heavy chain polypeptide comprising a structure represented by the formula:
VH₁-CH1-CH2-CH3 [I],
a first light chain polypeptide chain comprising a structure represented by the formula:
VL₁-CL₁ [II],
a second heavy chain polypeptide comprising a structure represented by the formula:
VH₂-CH1-CH2-CH3 [III],
and a second light chain polypeptide chain comprising a structure represented by the formula:
VL₂-CL₂ [IV];
wherein:
VL₁ is a first immunoglobulin light chain variable domain;
VL₂ is a second immunoglobulin light chain variable domain;
CL₁ is a first immunoglobulin light chain constant domain;
CL₂ is a second immunoglobulin light chain constant domain;
VH₁ is a first immunoglobulin heavy chain variable domain;
VH₂ is a second immunoglobulin heavy chain variable domain;
CH1 is an immunoglobulin CH1 heavy chain constant domain,
CH2 is an immunoglobulin CH2 heavy chain constant domain and
CH3 is an immunoglobulin CH3 heavy chain constant domain;
wherein CL₁ comprises one or more amino acid substitutions which reduce binding to a KappaSelect chromatography material compared to a CL₁ without the one or more amino acid substitutions, wherein VL₂ comprises one or more amino acid substitutions which reduce binding to a protein L chromatography material compared to a VL₂ without the one or more amino acid substitutions, and
wherein VH₁ and VL₁ associate to form a first antigen binding domain and VH₂ and VL₂ associate to form a second antigen binding domain.

3-33. (canceled)

34: A multivalent binding protein comprising four polypeptide chains that form three antigen binding domains; wherein the four polypeptide chains comprise:
a first heavy chain polypeptide comprising a structure represented by the formula:
VH₁-L₃-VH₂-L₄-CH1 [I],
a first light chain polypeptide chain comprising a structure represented by the formula:
VL₂-L₁-VL₁-L₂-CL₁ [II],
a second heavy chain polypeptide comprising a structure represented by the formula:
VH₃-CH1 [III],
and a second light chain polypeptide chain comprising a structure represented by the formula:
VL₃-CL₂ [IV]
wherein:
VL₁ is a first immunoglobulin light chain variable domain;
VL₂ is a second immunoglobulin light chain variable domain;
VL₃ is a third immunoglobulin light chain variable domain;
VH₁ is a first immunoglobulin heavy chain variable domain;
VH₂ is a second immunoglobulin heavy chain variable domain;
VH₃ is a third immunoglobulin heavy chain variable domain;
CL₁ is a first immunoglobulin light chain constant domain;
CL₂ is a second immunoglobulin light chain constant domain;
CH1 is an immunoglobulin CH₁ heavy chain constant domain; and
L₁, L₂, L₃ and L₄ are amino acid linkers;
wherein the polypeptide of formula I and the polypeptide of formula II form a cross-over light chain-heavy chain pair;
wherein VH₁ and VL₁ associate to form a first antigen binding domain, VH₂ and VL₂ associate to form a second antigen binding domain, and VH₃ and VL₃ associate to form a third antigen binding domain; and
wherein:
a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype immunoglobulin light chain variable domain; or
d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype immunoglobulin light chain variable domain.

35: A multivalent binding protein comprising four polypeptide chains that form three antigen binding domains; wherein the four polypeptide chains comprise:
a first heavy chain polypeptide comprising a structure represented by the formula:
VH₁-L₃-VH₂-L₄-CH1-CH2-CH3 [Ia],
a first light chain polypeptide chain comprising a structure represented by the formula:
VL₂-L₁-VL₁-L₂-CL₁ [II],
a second heavy chain polypeptide comprising a structure represented by the formula:
VH₃-CH1-CH2-CH3 [IIIa],
and a second light chain polypeptide chain comprising a structure represented by the formula:
VL₃-CL₂ [IV]
wherein:
VL₁ is a first immunoglobulin light chain variable domain;
VL₂ is a second immunoglobulin light chain variable domain;
VL₃ is a third immunoglobulin light chain variable domain;
VH₁ is a first immunoglobulin heavy chain variable domain;
VH₂ is a second immunoglobulin heavy chain variable domain;
VH₃ is a third immunoglobulin heavy chain variable domain;
CL₁ is a first immunoglobulin light chain constant domain;
CL₂ is a second immunoglobulin light chain constant domain;
CH1 is an immunoglobulin CH1 heavy chain constant domain;
CH2 is an immunoglobulin CH2 heavy chain constant domain;
CH3 is an immunoglobulin CH3 heavy chain constant domain; and
L₁, L₂, L₃ and L₄ are amino acid linkers;
wherein the polypeptide of formula I and the polypeptide of formula II form a cross-over light chain-heavy chain pair;
wherein VH₁ and VL₁ associate to form a first antigen binding domain, VH₂ and VL₂ associate to form a second antigen binding domain, and VH₃ and VL₃ associate to form a third antigen binding domain; and
wherein:
a) CL₁ comprises one or more amino acid substitutions which reduce binding to a KappaSelect chromatography material compared to a CL₁ without the one or more amino acid substitutions and VL₃ comprises one or more amino acid substitutions which reduce binding to a protein L chromatography material compared to a VL₃ without the one or more amino acid substitutions;
b) CL₂ comprises one or more amino acid substitutions which reduce binding to a KappaSelect chromatography material compared to a CL₂ without the one or more amino acid substitutions and VL₁ and VL₂ each comprise one or more amino acid substitutions which reduce binding to a protein L chromatography material compared to a VL₁ and VL₂ without the one or more amino acid substitutions;
c) CL₂ comprises one or more amino acid substitutions which reduce binding to a KappaSelect chromatography material compared to a CL₂ without the one or more amino acid substitutions, VL₁ comprises one or more amino acid substitutions which reduce binding to a protein L chromatography material compared to a VL₁ without the one or more amino acid substitutions, and wherein VL₂ is a λ subtype immunoglobulin light chain variable domain or a κ2 subtype light chain variable immunoglobulin domain; or
d) CL₂ comprises one or more amino acid substitutions which reduce binding to a KappaSelect chromatography material compared to a CL₂ without the one or more amino acid substitutions, VL₂ comprises one or more amino acid substitutions which reduce binding to a protein L chromatography material compared to a VL₂ without the one or more amino acid substitutions, and wherein VL₁ is a λ subtype immunoglobulin light chain variable domain or a κ2 subtype light chain variable immunoglobulin domain.

36: The multivalent binding protein of claim 35, wherein the binding protein is trispecific and capable of specifically binding three different antigen targets.

37-45. (canceled)

46: The multivalent binding protein of claim 35, wherein the one or more amino acid substitutions in the CL₁ or the CL₂ is at a position corresponding to 109, 110 or 199, wherein numbering is according to the EU index.

47: The multivalent binding protein of claim 46, wherein the one or more amino acid substitutions in the CL₁ or the CL₂ that comprises the one or more amino acid substitutions is a T109A substitution, a V110D substitution, a Q199K substitution, T109A-V110D substitutions, or T109A-V110D-Q199K substitutions, wherein amino acid numbering is according to the EU index.

48: The multivalent binding protein of claim 35, wherein the one or more amino acid substitutions in the CL₁ or the CL₂ that comprises the one or more substitutions is at a position corresponding to 109, 198, 199, or 202, wherein numbering is according to the EU index.

49: The multivalent binding protein of claim 48, wherein the one or more amino acid substitutions in the CL₁ or the CL₂ that comprises the one or more substitutions is a H198R substitution, a Q199W substitution, or T109A-S202R substitutions, wherein amino acid numbering is according to the EU index.

50: The multivalent binding protein of claim 35, wherein the one or more amino acid substitutions in the VL₁, the VL₂, the VL₃ and/or the VL₄ that comprises the one or more substitutions is a substitution of a framework amino acid.

51: The multivalent binding protein of claim 35, wherein the one or more amino acid substitutions in the VL₁, the VL₂, the VL₃ and/or the VL₄ that comprises the one or more substitutions is at a position corresponding to 12 or 18, wherein numbering is according to Kabat.

52: The multivalent binding protein of claim 51, wherein the one or more amino acid substitutions in the VL₁, the VL₂, the VL₃ and/or the VL₄ that comprises the one or more substitutions is a S12P substitution, a R18P substitution, a R18Q substitution, S12P-R18P substitutions, or S12P-R18Q substitutions, wherein numbering is according to Kabat.

53-60. (canceled)

61: A multivalent binding protein comprising four polypeptide chains that form two antigen binding domains; wherein the four polypeptide chains comprise:
a first heavy chain polypeptide comprising a structure represented by the formula:
VH₁-CH1-CH2-CH3 [I],
a first light chain polypeptide chain comprising a structure represented by the formula:
VL₁-CL₁ [II],
a second heavy chain polypeptide comprising a structure represented by the formula:
VH₂-CL₂-CH2-CH3 [III],
and a second light chain polypeptide chain comprising a structure represented by the formula:
VL₂-CH1 [IV];
wherein:
VL₁ is a first immunoglobulin light chain variable domain;
VL₂ is a second immunoglobulin light chain variable domain;
CL₁ is a first immunoglobulin light chain constant domain;
CL₂ is a second immunoglobulin light chain constant domain;
VH₁ is a first immunoglobulin heavy chain variable domain;
VH₂ is a second immunoglobulin heavy chain variable domain;
CH1 is an immunoglobulin heavy chain constant domain;
CH2 is an immunoglobulin CH2 heavy chain constant domain; and
CH3 is an immunoglobulin CH3 heavy chain constant domain
wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

62. (canceled)

63: A multivalent binding protein comprising four polypeptide chains that form two antigen binding domains; wherein the four polypeptide chains comprise:
a first heavy chain polypeptide comprising a structure represented by the formula:
VH₁-CH1-CH2-CH3 [I],
a first light chain polypeptide chain comprising a structure represented by the formula:
VL₁-CL₁ [II],
a second heavy chain polypeptide comprising a structure represented by the formula:
VH₂-CH1-CH2-CH3 [III],
and a second light chain polypeptide chain comprising a structure represented by the formula:
VL₂-CL₂ [IV];
wherein:
VL₁ is a first immunoglobulin light chain variable domain;
VL₂ is a second immunoglobulin light chain variable domain;
CL₁ is a first immunoglobulin light chain constant domain;
CL₂ is a second immunoglobulin light chain constant domain;
VH₁ is a first immunoglobulin heavy chain variable domain;
VH₂ is a second immunoglobulin heavy chain variable domain;
CH1 is an immunoglobulin heavy chain constant domain;
CH2 is an immunoglobulin CH2 heavy chain constant domain; and
CH3 is an immunoglobulin CH3 heavy chain constant domain;
wherein
a) CL₂ comprises one or more amino acid substitutions which reduce binding to a KappaSelect chromatography material compared to a CL₂ without the one or more amino acid substitutions and VL₁ comprises one or more amino acid substitutions which reduce binding to a protein L chromatography material compared to a VL₁ without the one or more amino acid substitutions, or
b) CL₁ comprises one or more amino acid substitutions which reduce binding to a KappaSelect chromatography material compared to a CL₁ without the one or more amino acid substitutions and VL₂ comprises one or more amino acid substitutions which reduce binding to a protein L chromatography material compared to a VL₂ without the one or more amino acid substitutions;
and wherein VH₁ and VL₁ associate to form a first antigen binding domain and VH₂ and VL₂ associate to form a second antigen binding domain.

64-81. (canceled)

82: A multivalent binding protein comprising four polypeptide chains that form four antigen binding domains; wherein the four polypeptide chains comprise:
a first heavy chain polypeptide comprising a structure represented by the formula:
VH₁-CH1₁-L₁-VH₂-CH1₂ [I],
a first light chain polypeptide comprising a structure represented by the formula:
VL₁-CL₁-L₂-VL₂-CL₂ [II],
a second heavy chain polypeptide comprising a structure represented by the formula:
VH₃-CH1₃-L₃-VH₄-CH1₄ [III],
a second light chain polypeptide comprising a structure represented by the formula:
VL₃-CL₃-L₄-VL₄-CL₄ [IV]
wherein:
VL₁ is a first immunoglobulin light chain variable domain;
VL₂ is a second immunoglobulin light chain variable domain;
VL₃ is a third immunoglobulin light chain variable domain;
VL₄ is a fourth immunoglobulin light chain variable domain;
CL₁ is a first immunoglobulin light chain constant domain;
CL₂ is a second immunoglobulin light chain constant domain;
CL₃ is a third immunoglobulin light chain constant domain;
CL₄ is a fourth immunoglobulin light chain constant domain;
VH₁ is a first immunoglobulin heavy chain variable domain;
VH₂ is a second immunoglobulin heavy chain variable domain;
VH₃ is a third immunoglobulin heavy chain variable domain;
VH₄ is a fourth immunoglobulin heavy chain variable domain;
CH1₁ is a first immunoglobulin heavy chain constant domain;
CH1₂ is a second immunoglobulin heavy chain constant domain;
CH1₃ is a third immunoglobulin heavy chain constant domain;
CH1₄ is a fourth immunoglobulin heavy chain constant domain;
and
L₁, L₂, L₃ and L₄ are amino acid linkers;
wherein:
a) CL₁ and CL₂ each comprise one or more amino acid substitutions which reduce binding to a KappaSelect chromatography material compared to a CL₁ and CL₂ without the one or more amino acid substitutions and VL₃ and VL₄ each comprise one or more amino acid substitutions which reduce binding to a protein L chromatography material compared to a VL₃ and VL₄ without the one or more amino acid substitutions;
b) CL₁ and CL₂ each comprise one or more amino acid substitutions which reduce binding to a KappaSelect chromatography material compared to a CL₁ and CL₂ without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
c) CL₁ and CL₂ each comprises one or more amino acid substitutions which reduce binding to a KappaSelect chromatography material compared to a CL₁ and CL₂ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a λ2 subtype immunoglobulin light chain variable domain;
d) CL₁ comprises one or more amino acid substitutions which reduce binding to a KappaSelect chromatography material compared to a CL₁ without the one or more amino acid substitutions, CL₂ is a λ subtype immunoglobulin light chain constant domain, and VL₃ and VL₄ each comprise one or more amino acid substitutions which reduce binding to a protein L chromatography material compared to a VL₁ and VL₂ without the one or more amino acid substitutions;
e) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
f) CL₁ comprises one or more amino acid substitutions which reduce binding to a KappaSelect chromatography material compared to a CL₁ without the one or more amino acid substitutions, CL₂ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
g) CL₂ comprises one or more amino acid substitutions which reduce binding to a KappaSelect chromatography material compared to a CL₂ without the one or more amino acid substitutions, CL₁ is a λ subtype immunoglobulin light chain constant domain, and VL₃ and VL₄ each comprise one or more amino acid substitutions which reduce binding to a protein L chromatography material compared to a VL₃ and VL₄ without the one or more amino acid substitutions;
h) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
i) CL₂ comprises one or more amino acid substitutions which reduce binding to a KappaSelect chromatography material compared to a CL₂ without the one or more amino acid substitutions, CL₁ is a λ subtype immunoglobulin light chain constant domain, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain.

83-84. (canceled)

85: A multivalent binding protein comprising four polypeptide chains that form four antigen binding domains; wherein the four polypeptide chains comprise:
a first heavy chain polypeptide comprising a structure represented by the formula:
VH₁-L₁-VH₂-L₂-CH1₁ [I],
a first light chain polypeptide comprising a structure represented by the formula:
VL₁-L₃-VL₂-L₄-CL₁ [II],
a second heavy chain polypeptide comprising a structure represented by the formula:
VH₃-L₅-VH₄-L₆-CH1₂ [III],
a second light chain polypeptide comprising a structure represented by the formula:
VL₃-L₇-VL₄-L₈-CL₂ [IV]
wherein:
VL₁ is a first immunoglobulin light chain variable domain;
VL₂ is a second immunoglobulin light chain variable domain;
VL₃ is a third immunoglobulin light chain variable domain;
VL₄ is a fourth immunoglobulin light chain variable domain;
CL₁ is a first immunoglobulin light chain constant domain;
CL₂ is a second immunoglobulin light chain constant domain;
VH₁ is a first immunoglobulin heavy chain variable domain;
VH₂ is a second immunoglobulin heavy chain variable domain;
VH₃ is a third immunoglobulin heavy chain variable domain;
VH₄ is a fourth immunoglobulin heavy chain variable domain;
CH1₁ is a first immunoglobulin heavy chain constant domain;
CH1₂ is a second immunoglobulin heavy chain constant domain;
and
L₁, L₂, L₃, L₄ L₅, L₆, L₇ and L₈ are amino acid linkers;
wherein:
a) CL₁ comprises one or more amino acid substitutions which reduce binding to a KappaSelect chromatography material compared to a CL₁ without the one or more amino acid substitutions and VL₃ and VL₄ each comprise one or more amino acid substitutions which reduce binding to a protein L chromatography material compared to a VL₃ and VL₄ without the one or more amino acid substitutions;
b) CL₁ comprises one or more amino acid substitutions which reduce binding to a KappaSelect chromatography material compared to a CL₁ without the one or more amino acid substitutions, VL₃ comprises one or more amino acid substitutions which reduce binding to a protein L chromatography material compared to a VL₃ without the one or more amino acid substitutions, and VL₄ is a λ subtype immunoglobulin light chain variable domain or a κ2 subtype immunoglobulin light chain variable domain;
c) CL₁ comprises one or more amino acid substitutions which reduce binding to a KappaSelect chromatography material compared to a CL₁ without the one or more amino acid substitutions, VL₄ comprises one or more amino acid substitutions which reduce binding to a protein L chromatography material compared to a VL₄ without the one or more amino acid substitutions, and wherein VL₃ is a λ subtype immunoglobulin light chain variable domain or a κ2 subtype immunoglobulin light chain variable domain;
and wherein VL₁ and VH₁ form a first antigen binding domain, VL₂ and VH₂ form a second antigen binding domain, VL₃ and VH₃ form a third antigen binding domain, and VL₄ and VH₄ form a fourth antigen binding domain.

86-109. (canceled)

110: A multivalent binding protein comprising four polypeptide chains that form two antigen binding domains; wherein the four polypeptide chains comprise:
a first heavy chain polypeptide comprises a structure represented by the formula:
VH₁-CH1₁-L₁-VH₂-CH1₂ [I],
a first light chain polypeptide chain comprises a structure represented by the formula:
VL₁-CL₁-L₂-VL₂-CL₂ [II],
a second heavy chain polypeptide comprises a structure represented by the formula:
fusion polypeptide-L₃-CH1₃ [III],
and a second light chain polypeptide comprises a structure represented by the formula:
fusion polypeptide-L₄-CL₃ [IV]
wherein:
VL₁ is a first immunoglobulin light chain variable domain;
VL₂ is a second immunoglobulin light chain variable domain;
CL₁ is a first immunoglobulin light chain constant domain;
CL₂ is a second immunoglobulin light chain constant domain;
CL₃ is a third immunoglobulin light chain constant domain;
VH₁ is a first immunoglobulin heavy chain variable domain;
VH₂ is a second immunoglobulin heavy chain variable domain;
CH1₁ is a first immunoglobulin heavy chain constant domain;
CH1₂ is a second immunoglobulin heavy chain constant domain;
CH1₃ is a third immunoglobulin heavy chain constant domain, and
L₁, L₂, L₃ and L₄ are amino acid linkers;
wherein:
a) CL₃ comprises one or more amino acid substitutions which reduce binding to a KappaSelect chromatography material compared to a CL₃ without the one or more amino acid substitutions, VL₁ and VL₂ each comprise one or more amino acid substitutions which reduce binding to a protein L chromatography material compared to a VL₁ and VL₂ without the one or more amino acid substitutions,
b) CL₃ comprises one or more amino acid substitutions which reduce binding to a KappaSelect chromatography material compared to a CL₃ without the one or more amino acid substitutions, VL₁ comprises one or more amino acid substitutions which reduce binding to a protein L chromatography material compared to a VL₁ without the one or more amino acid substitutions, and VL₂ is a λ subtype immunoglobulin light chain variable domain or a κ2 subtype immunoglobulin domain; or
c) CL₃ comprises one or more amino acid substitutions which reduce binding to a KappaSelect chromatography material compared to a CL₃ without the one or more amino acid substitutions, VL₂ comprises one or more amino acid substitutions which reduce binding to a protein L chromatography material compared to a VL₂ without the one or more amino acid substitutions, and VL₁ is a λ subtype immunoglobulin light chain variable domain or a κ2 subtype immunoglobulin domain; and
wherein VL₁ and VH₁ form a first antigen binding domain and VL₂ and VH₂ form a second antigen binding domain.

111-129. (canceled)

130: A polynucleotide encoding the multivalent binding protein of claim 35.

131: A vector comprising the polynucleotide of claim 130.

132: A host cell comprising the polynucleotide of claim 130.

133: A method of producing a multivalent binding protein, the method comprising culturing the host cell of claim 132 such that the binding protein is produced.

134. (canceled)

135: A pharmaceutical composition comprising the multivalent binding protein of claim 35 and a pharmaceutically acceptable carrier.

136: A method of purifying the multivalent binding protein of claim 35, the method comprising
a) subjecting a composition comprising the multivalent binding protein to Protein L chromatography in bind and elute mode to generate a protein L eluate, and
b) subjecting the protein L eluate to KappaSelect chromatography in bind and elute mode to generate a KappaSelect eluate,
wherein the KappaSelect eluate comprises the multivalent binding protein and is essentially free of mispaired polypeptides.

137-138. (canceled)

139: A method of purifying the multivalent binding protein of claim 35, the method comprising
a) subjecting a composition comprising the multivalent binding protein and mispaired antibodies to KappaSelect chromatography in bind and elute chromatography to generate as KappaSelect eluate and
b) subjecting the KappaSelect eluate to Protein L chromatography in bind and elute mode to generate a protein L eluate,
wherein the protein L eluate comprises the multivalent binding protein and is essentially free of mispaired polypeptides.

140-141. (canceled)

142: A method of purifying the multivalent binding protein of claim 35, the method comprising
a) subjecting a composition comprising the multivalent binding protein to Protein A chromatography in bind and elute mode to generate a Protein A eluate,
b) subjecting the Protein A eluate to Protein L chromatography in bind and elute mode to generate a protein L eluate, and
c) subjecting the protein L eluate to KappaSelect chromatography in bind and elute mode to generate a KappaSelect eluate,
wherein the KappaSelect eluate comprises the multivalent binding protein and is essentially free of mispaired polypeptides.

143-144. (canceled)

145: A method of purifying the multivalent binding protein of claim 35, the method comprising
a) subjecting a composition comprising the multivalent binding protein to Protein A chromatography in bind and elute mode to generate a Protein A eluate,
b) subjecting the Protein A eluate to KappaSelect chromatography in bind and elute mode to generate a KappaSelect eluate, and
c) subjecting the protein KappaSelect eluate to Protein L chromatography in bind and elute mode to generate a protein L eluate,
wherein the L eluate comprises the multivalent binding protein and is essentially free of mispaired polypeptides.

146-157. (canceled)
